(19) United States
(12) Patent Application Publication
(10) Pub. No.: US 2023/0159467 A1
(43) Pub. Date: May 25, 2023

(54) NOVEL COMPOUNDS USEFUL IN THE TREATMENT AND/OR PREVENTION OF A DISEASE, DISORDER OR CONDITION ASSOCIATED WITH ANGIOTENSIN II

(71) Applicant: Vicore Pharma AB, Stockholm (SE)

(72) Inventors: Anders Hallberg, Uppsala (SE); Mats Larhed, Uppsala (SE)

(21) Appl. No.: 17/912,284

(22) PCT Filed: Mar. 18, 2021
(86) PCT No.: PCT/GB2021/050680

§ 371 Date: Sep. 16, 2022

(30) Foreign Application Priority Data
Mar. 19, 2020 (SE) 2050301-7
Jun. 29, 2020 (SE) 2050782-8

(51) Int. Cl. C07D 233/64; C07D 417/10; C07D 409/10; C07D 417/14; C07D 417/04; C07D 413/10

(52) U.S. Cl. CPC C07D 233/64; C07D 417/10; C07D 409/10; C07D 417/14; C07D 417/04; C07D 413/10

(57) ABSTRACT

The present disclosure relates to compounds of Formula (I) and to their utility in the treatment in a disease, disorder and/or condition associated with the peptide angiotensin II.

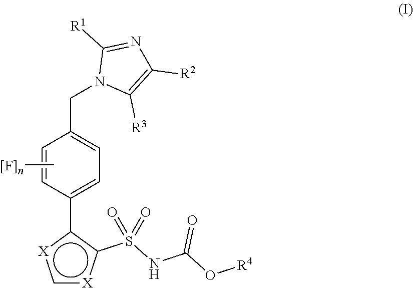

### TECHNICAL FIELD

The present disclosure relates to novel compounds, to their use as pharmaceutical drugs that are useful in the treatment and/or prevention of a disease, disorder and/or condition associated with the peptide angiotensin II, and to processes for preparing such compounds.

### BACKGROUND

The renin-angiotensin system (RAS) is a hormone system that regulates blood pressure, fluid and electrolyte balance, as well as systemic vascular resistance. The renin-angiotensin system (RAS) includes angiotensinogen (AGT), renin, angiotensin-converting enzyme (ACE), angiotensin II (Ang II) and the two Ang II receptors AT1 and AT2. Angiotensinogen, which is a protein with 452 amino acids, is cleaved by the enzyme renin to produce the peptide Angiotensinogen I (Ang I), which is then cleaved by angiotensin-converting enzyme ACE to Ang II. Ang II acts as an agonist by activating the receptors AT1 and AT2 thereby regulating physiological and pathophysiological conditions. Importantly, Ang II provides a strong hypertensive effect mediated by the AT1 receptor and modulates many cardiovascular functions.

The central role of the ACE and renin enzymes in the renin-angiotensin system (RAS) has been a starting point for structure based drug design and resulted in pharmaceutical drugs that inhibit these enzymes. The pharmaceutical drug Losartan acting as a selective antagonist on the AT1 receptor was introduced on the market in 1995 and was followed by many other “sartan” drugs such as valsartan, candesartan and irbesartan. The renin inhibitor aliskiren was approved for the treatment of hypertension in the US in 2007.

It has also been proposed that agonists acting selectively at the AT2 receptor may be used for treating disorders associated with the renin-angiotensin system (RAS), and that activation of the AT2 receptor frequently has opposing effects to those mediated by the AT1 receptor. The AT1 receptor is expressed in most organs of the human body while the AT2 receptor has been found in fetal tissues with the expression considerably lowered after birth. However, the expression of the AT2 receptor is up-regulated in pathological conditions such as heart failure, renal failure, myocardial infarction, brain lesions, vascular injury and wound healing.

The AT2 receptor has also been shown to be involved in apoptosis and inhibition of cell proliferation (Pharmacol. Rev. 2000; 52: 415-472).

WO 02/096883 discloses tricyclic compounds useful as selective agonists at the AT2 receptor. The compounds may include an imidazole moiety. The compounds may be used in the treatment of gastrointestinal conditions such as dyspepsia, Inflammatory bowel disease (IBS) and multiple organ failure (MOF) (see, for example, international patent application WO 99/43339), and cardiovascular disorders.

EP 0512675 A1 discloses that substituted imidazoles attached through a methylene to novel substituted phenyl thiophene or phenyl furan derivative are angiotensin II antagonists and are useful in the treatment of hypertension and congestive heart failure.

DE10 2012 004 589 A1 discloses new angiotensin II receptor agonists useful for treating neurodegenerative diseases, preferably Alzheimer's dementia, Parkinson 's disease, Huntington's disease and amyotrophic lateral sclerosis.

US patent application US 2004/0167176 describes the preparation of tricyclic heterocycles useful as angiotensin II receptor agonists.

Transesterification methods for synthesis of AT2 receptor ligands with improved stability in human liver microsomes are described in Bioorg. Med. Chem. Lett. 2018; 28:519-522.

Med. Res. Rev. 2018; 38:602-624 discloses small-molecule AT2 receptor agonists. The discovery of the selective small-molecule AT2 receptor agonist compound 21 C21 is described (see also international patent application WO 2002/096883). C21 is now in clinical development for treatment of AT2 receptor related disorders, including IPF (see, for example, international patent application WO 2016/139475).

C21 has also been indicated to be of potential use in the treatment of inter alia, stroke, spinal cord injury, sickle cell disease, muscular dystrophy, cancer treatment-related cardiotoxicity, peripheral neuropathy and systemic sclerosis (see, for example, international patent applications WO 2004/046141, WO 2016/092329, WO 2016/107879, WO 2016/139475, WO 2017/221012, WO 2019/008393, and US patent application US 2012/035232).

The compound C21 is reported to inhibit a series of oxidative enzymes from the CYP family, and that substitution of the imidazole ring of C21 with a methyl group or a trifluoromethyl group reduced the level of inhibition of CYP 3A4 and 2C9. Further, a minor structural alteration of C21 converted it from being a selective AT2 receptor agonist into the selective AT2 receptor antagonist C38/M132.

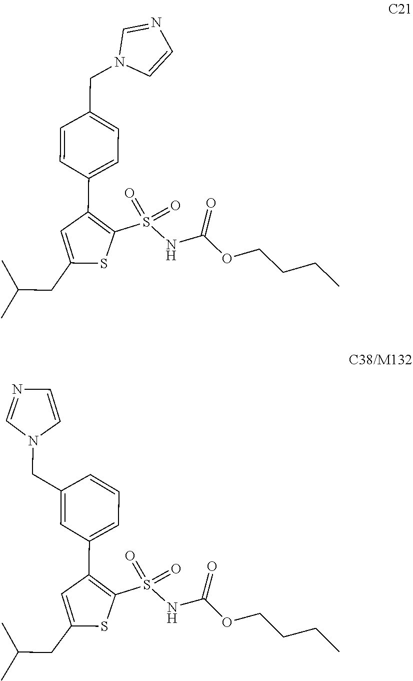

Cytochromes P450 (CYPs) are a family of enzymes present in e.g. mammals where they are important for the clearance of various compounds such as pharmaceutical drugs. Therefore, drug interactions with various CYPs are important to consider in order to avoid undesired side effects. A too large extent of CYP inhibition may potentially negatively impact the usefulness of a compound as a pharmaceutical drug.

Selective AT2 receptor agonists with reduced CYP inhibition are described in Bioorg. Med. Chem. 2010; 18:4570-4590.

There remains a need for a pharmaceutically-active compounds, such as small molecules that act as AT2 receptor agonists for treating and/or preventing a disease, disorder or condition associated with angiotensin II. In particular, there is a need for such a pharmaceutical drug wherein said pharmaceutical drug exhibits an acceptable level of CYP inhibition of one or more CYPs.

### SUMMARY OF THE INVENTION

The present disclosure provides a pharmaceutical drug such as a small molecule acting as an AT2 receptor agonist for treating and/or preventing a disease, disorder or condition associated with angiotensin II. Said pharmaceutical drug may exhibit an acceptable level of CYP inhibition of one or more CYPs.

Further, the present disclosure provides a pharmaceutical drug such as a small molecule acting as selective AT2 receptor agonist exhibiting an acceptable level of CYP inhibition of one or more CYPs.

In this respect, there is provided a compound of Formula I:

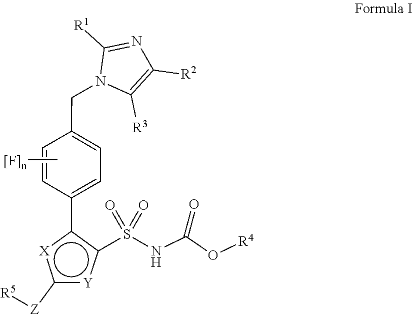

or a pharmaceutically acceptable salt thereof, wherein, in the compound of Formula I, R¹ represents

H;

C₂-C₆ alkyl substituted with 0, 1 or 2 substituents (i.e. optionally substituted with 1 or 2 substituents) selected from the group consisting of OR⁶, SR⁷, NR⁸R⁹, halogen, thiazole, oxazole and pyrazole;

C₃-C₆ cycloalkyl substituted with 0, 1 or 2 substituents (i.e. optionally substituted with 1 or 2 substituents) selected from the group consisting of OR⁶, SR⁷ and NR⁸R⁹; thiazole;

benzyl wherein the phenyl moiety thereof is substituted with 0, 1 or 2 substituents (i.e. optionally substituted with 1 or 2 substituents) selected from the group consisting of OR⁶, SR⁷ and NR⁵R⁹; or

(CH₂)ₘ—R¹⁰,

R² represents

H;

F;

Cl;

C₂-C₆ alkyl substituted with 0, 1 or 2 substituents (i.e. optionally substituted with 1 or 2 substituents) selected from the group consisting of OR⁶, SR⁷, NR⁸R⁹, halogen, thiazole, oxazole and pyrazole;

C₃-C₆ cycloalkyl substituted with 0, 1 or 2 substituents (i.e. optionally substituted with 1 or 2 substituents) selected from the group consisting of OR⁶, SR⁷ and NR⁸R⁹; benzyl wherein the phenyl moiety thereof is substituted with 0, 1 or 2 substituents (i.e. optionally substituted with 1 or 2 substituents) selected from the group consisting of OR⁶, SR⁷ and NR⁸R⁹; or

(CH₂)ₘ—R¹⁰,

provided that R¹ and R² are not both H,

R³ represents

H;

halogen; or

C₁-C₃ alkyl substituted with 0, 1, 2 or 3 halogens (i.e. optionally substituted with 1, 2 or 3 halogens) selected from the group consisting of F and Cl,

R⁴ and R⁵ independently represent C₁-C₆ alkyl substituted with 0, 1, 2 or 3 F (i.e. optionally substituted with 1, 2 or 3 fluoro substituents),

X represents CH═CH, CH, N, NH, O or S; and

Y represents CH═CH, CH, N, NH, O or S, provided that:

(a) X and Y are not the same,

(b) when X represents CH=CH then Y may only represent CH, and

(c) when Y represents CH=CH then X may only represent CH,

Z represents a single bond, O or S;

R⁶, R⁷, R⁸ and R⁹ independently represent

H or C₁-C₃ alkyl substituted with 0, 1, 2 or 3 F (i.e. optionally substituted with 1, 2 or 3 fluoro substituents),

R¹⁰ is selected from the group consisting of phenyl, thiazole, oxazole and pyrazole;

n is 0, 1, 2, 3 or 4; and

m is 0 or 1.

The aforementioned compounds (including pharmaceutically acceptable salts thereof) that are disclosed herein are referred to interchangeably hereinafter as “compounds of the invention”, “compounds of the present disclosure”, “compounds as described herein”, or “compounds of Formula I”.

There is also provided a pharmaceutical composition comprising a therapeutically effective amount of a compound of Formula I as described herein, or a pharmaceutically acceptable salt thereof, together with at least one pharmaceutically acceptable carrier, excipient and/or diluent.

Further, there is provided a compound of Formula I as described herein, or a pharmaceutically acceptable salt thereof, or a pharmaceutical composition as described herein for use as a medicament. The medicament may be a medicament for the treatment and/or prevention of a disease, disorder and/or condition associated with the peptide angiotensin II.

There is also provided a compound of Formula I as described herein, or a pharmaceutically acceptable salt thereof, or a pharmaceutical composition as described herein for use in the treatment and/or prevention of a disease, disorder and/or condition selected from the group consisting of obstructive lung diseases, such as chronic obstructive lung disease, autoimmune diseases, such as rheumatoid arthritis, viral respiratory tract infections, such as pneumonia resulting from a respiratory viral infection (viral pneumonia) and, more preferably, hypertension, heart failure, stroke, chronic kidney disease (including nephropathy), pulmonary fibrosis such as idiopathic pulmonary fibrosis, sclerosis such as systemic sclerosis, sarcoidosis such as pulmonary sarcoidosis, and other conditions including one or more of those not described hereinbefore but listed hereinafter, and any combination thereof.

There is also provided a use of a compound of Formula I as described herein, or a pharmaceutically acceptable salt thereof, or a pharmaceutical composition as described herein for the manufacture of a medicament for the treatment and/or prevention of a disease, disorder and/or condition selected from the group consisting of hypertension, heart failure, stroke, nephropathy, pulmonary fibrosis such as idiopathic pulmonary fibrosis, sclerosis such as systemic sclerosis, sarcoidosis such as pulmonary sarcoidosis, and other conditions including one or more of those not described hereinbefore but listed hereinafter, and any combination thereof.

There is also provided a method for treatment and/or prevention of a disease, disorder and/or condition selected from the group consisting of hypertension, heart failure, stroke, nephropathy, pulmonary fibrosis such as idiopathic pulmonary fibrosis, sclerosis such as systemic sclerosis, sarcoidosis such as pulmonary sarcoidosis, and other conditions including one or more of those not described hereinbefore but listed hereinafter, and any combination thereof, which method comprises the step of administering a therapeutically effective amount of a compound of Formula I as described herein or a pharmaceutical composition as described herein to a patient in need thereof.

### DETAILED DESCRIPTION OF THE INVENTION

The present disclosure provides a compound of Formula I:

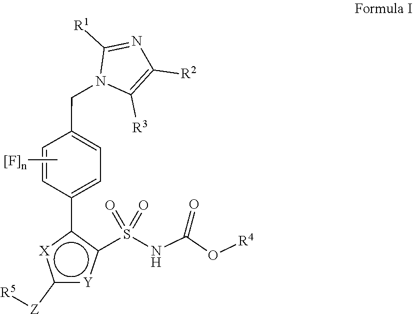

or a pharmaceutically acceptable salt thereof, as hereinbefore defined.

For the avoidance of doubt, the skilled person will understand that references herein to compounds of particular aspects of the invention (such as any aspect of the invention referring to compounds of Formula I as defined hereinbefore) will include references to all embodiments and particular features thereof, which embodiments and particular features may be taken in combination to form further embodiments and features of the invention.

Unless indicated otherwise, all technical and scientific terms used herein will have their common meaning as understood by one of ordinary skill in the art to which this invention pertains.

The term “alkyl” denotes a straight or branched saturated or unsaturated alkyl chain. It is to be understood that when “alkyl” is an unsaturated alkyl chain, i.e. an alkene, it includes two or more carbon atoms.

The term “C₁-C₆ alkyl” denotes a straight-chain or, when there is a sufficient number (i.e. a minimum of two or three, as appropriate) of carbon atoms, be branched-chain, and/or cyclic (so forming a C₃₋₆ cycloalkyl group) saturated or unsaturated alkyl chain comprising from one to six carbon atoms. When there is a sufficient number (i.e. a minimum of four) of carbon atoms, such groups may also be part-cyclic (so forming a C₄₋₆ partial cycloalkyl group).

Examples of “C₂-C₆ alkyl” include, but are not limited to, methyl, ethyl, propyl, isopropyl, n-butyl, sec-butyl, isobutyl, tert-butyl, n-pentyl, isopentyl, neo-pentyl, n-hexyl, isohexyl, 3-methylpentyl, 2,3-dimethylbutyl, and neohexyl.

The term “C₂-C₆ alkyl” denotes a straight or branched saturated or unsaturated alkyl chain, as defined hereinbefore, comprising from two to six carbon atoms. Examples of “C₂-C₆ alkyl” include, but are not limited to, ethyl, propyl, isopropyl, n-butyl, sec-butyl, isobutyl, tert-butyl, n-pentyl, isopentyl, neo-pentyl, n-hexyl, isohexyl, 3-methylpentyl, 2,3-dimethylbutyl, and neohexyl.

The term “C₁-C₃ alkyl” denotes a straight or branched saturated or unsaturated alkyl chain, as defined hereinbefore, comprising from one to three carbon atoms. Examples of “C₁-C₃ alkyl” include, but are not limited to, ethyl, propyl, and isopropyl.

The term “C₃-C₆ cycloalkyl” denotes a saturated or unsaturated non-aromatic monocyclic ring, as defined hereinbefore, composed of three, four, five or six carbon atoms. Examples of “C₃-C₆ cycloalkyl” include, but are not limited to, cyclopropyl, cyclobutyl, cyclopentyl and cyclohexyl.

Examples of part-cyclic alkyl groups (which may also be referred to as “part-cycloalkyl” groups) that may be mentioned include cyclopropylmethyl. When there is a sufficient number of carbon atoms, such groups may also be multicyclic (e.g. bicyclic or tricyclic) and/or spirocyclic.

The term “halogen” includes fluoro, chloro, bromo and iodo.

It will be appreciated that when the R¹ substituent described herein represents, for example, thiazole it may bind to the imidazole ring of the compound of Formula I as follows:

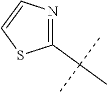

In cases in which the identity of two or more substituents in a compound of the invention may be the same, the actual identities of the respective substituents are not in any way interdependent. For example, in the situation in which two or more halo groups are present, those groups may be the same or different (e.g. two chloro groups, or a fluoro and a chloro group). Similarly, where two or more alkyl groups are present, the groups in question may be the same or different in terms of their number of carbon atoms and/or whether they are linear, branched, unsaturated or otherwise.

Unless otherwise specified, substituents may be located at any point on a group to which they may be attached. In this respect, alkyl groups (for example) that may be substituted by one or more substituents may also be terminated by such substituents (by which we mean located at the terminus of an e.g. alkyl chain).

For the avoidance of doubt, wherein it is stated that a substituent is itself substituted with 1 or more substituents (e.g. C₁-C₃ alkyl substituted with 1, 2 or 3 halogens), these substituents (e.g. halogens) where possible may be positioned on the same or different atoms.

The skilled person will appreciate that compounds of the invention that are the subject of this invention include those that are obtainable, i.e. those that may be prepared in a stable form. That is, compounds of the invention include those that are sufficiently robust to survive isolation, e.g. from a reaction mixture, to a useful degree of purity.

Compounds of Formula I may be made in accordance with techniques well known to those skilled in the art, for example as described hereinafter.

In one embodiment, the moiety containing X and Y of the compound of Formula I may be a phenyl ring thereby providing a compound of Formula Ia or Formula Ib:

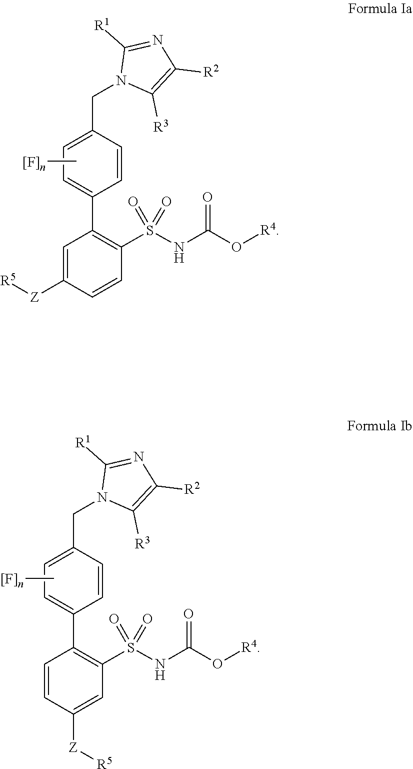

Alternatively, the moiety containing X and Y of the compound of Formula I may be a thiophene thereby providing a compound of Formula Ic.

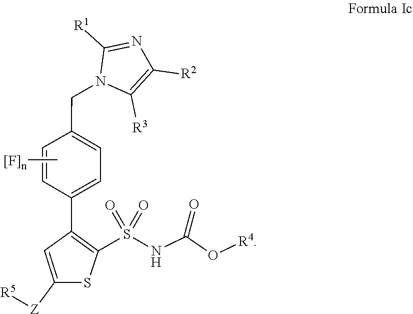

In another embodiment, the moiety containing X and Y of the compound of Formula I may be a thiazole thereby providing a compound of Formula Id.

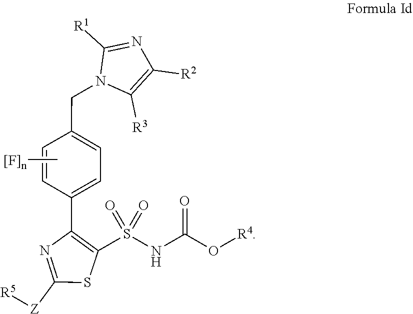

The compound of Formula I described herein may have the following values for R¹ and R²:

R¹ may represent C₂-C₆ alkyl substituted with 0, 1 or 2 substituents (i.e. optionally substituted with 1 or 2 substituents) selected from the group consisting of OR⁶, SR⁷, NR⁸R⁹, halogen, or a thiazole, oxazole or pyrazole, and

R² may represent H.

For instance, R¹ may be selected from the group consisting of ethyl, n-propyl, isopropyl, n-butyl, sec-butyl, isobutyl and tert-butyl, and R² may represent H.

In a further example, R¹ may represent C₂₋₆ alkyl (e.g. ethyl, propyl or butyl) optionally substituted by 1 or 2 substituents selected from OR⁶ or halogen, and R² may represent H.

For instance, R¹ may represent ethyl or propyl optionally substituted by an —OH group, for example 2-hydroxyprop-2-yl or 1-ethanol, and R² may represent H. R¹ may also represent ethyl or propyl optionally substituted by F, for example 2-fluoroprop-2-yl, and R² may represent H.

In another example, R¹ may represent C₂-C₆ alkyl groups, such as isopropyl or cyclopropyl, and R² may represent H.

In a yet another example, R¹ may represent thiazole and R² may represent H.

In a yet further example, R¹ may represent 2-hydroxyprop-2-yl or, more preferably, tert-butyl and R² may represent H.

Other compounds of the invention that may be mentioned include those in which:

R¹ may represent H, and

R² may represent C₂-C₆ alkyl substituted with 0, 1 or 2 substituents (i.e. optionally substituted with 1 or 2 substituents) selected from the group consisting of OR⁶, SR⁷, NR⁸R⁹, halogen, or thiazole, oxazole or pyrazole.

For instance, R¹ may represent H and R² may be selected from the group consisting of ethyl, n-propyl, isopropyl, n-butyl, sec-butyl, isobutyl and tert-butyl.

In a further example, R¹ may represent H and R² may represent tert-butyl.

The compound of Formula I described herein, may have a R⁴ substituent selected from the group consisting of methyl, ethyl, n-propyl, isopropyl, n-butyl, isobutyl, sec-butyl, and tert-butyl. For example, the R⁴ substituent may be tert-butyl, methyl, ethyl, n-propyl or n-butyl, such as n-butyl or, particularly, methyl.

Additionally or alternatively, the compound of Formula I described herein, may have a R⁵ substituent selected from the group consisting of ethyl, n-propyl, isopropyl, n-butyl, isobutyl, sec-butyl and tert-butyl. For example, the R⁵ substituent may be isopropyl or isobutyl.

For instance, there is provided a compound of Formula I described herein, wherein

R⁴ is selected from the group consisting of methyl, ethyl, n-propyl, isopropyl, n-butyl, isobutyl, sec-butyl, and tert-butyl, and

R⁵ is selected from the group consisting of ethyl, n-propyl, isopropyl, n-butyl, isobutyl, sec-butyl and tert-butyl.

Further, there is provided a compound of Formula I described herein, wherein

R⁴ is methyl, and

R⁵ is isopropyl or isobutyl.

In a further example, there is provided a compound of Formula I described herein,

wherein

R⁴ is methyl, and

R⁵ is n-propyl.

In a yet further example, there is provided a compound of Formula I described herein,

wherein

R⁴ is n-butyl, and

R⁵ is isopropyl or isobutyl.

In another example, there is provided a compound of Formula I described herein,

wherein

R⁴ is n-butyl, and

R⁵ is n-propyl.

Preferred compounds of the invention include compounds of any one of Formulae Ia, Ib, Ic or Id, in which:

R¹ represents C₂₋₄ alkyl optionally substituted by one of more substituent selected from OR⁶ or halogen;

R² and R³ both represent H;

R⁴ represents C1-2 alkyl;

R⁵ represents isopropyl or isobutyl;

n represents 0.

More preferred compounds of the invention include compounds of any one of formulae Ic or, more preferably, Ia, in which:

R¹ represents a linear or branched propyl or butyl group optionally substituted by OH or fluoro, for example R¹ represents tert-butyl or 2-hydroxyprop-2-yl;

R⁴ represents methyl;

R⁵ represents isopropyl.

The present disclosure also provides a compound of Formula I as described herein, wherein the phenyl ring binding to the nitrogen atom of the imidazole moiety via a methylene group may be unsubstituted. Thus, the value for “n” may be 0.

The present disclosure provides a compound which is one or more of the following:

methyl ((4′-((2-(tert-butyl)-1H-imidazol-1-yl)methyl)-5-isobutyl-[1,1′-biphenyl]-2-yl)sulfonyl)carbamate,

butyl ((4′-((2-(tert-butyl)-1H-imidazol-1-yl)methyl)-5-isobutyl-[1,1′-biphenyl]-2-yl)sulfonyl)carbamate,

methyl ((4-(4-((2-(tert-butyl)-1H-imidazol-1-yl)methyl)phenyl)-2-isobutylthiazol-5-yl)sulfonyl) carbamate,

butyl ((4-(4-((2-(tert-butyl)-1H-imidazol-1-yl)methyl)phenyl)-2-isobutylthiazol-5-yl)sulfonyl) carbamate,

methyl ((4-(4-((2-(tert-butyl)-1H-imidazol-1-yl)methyl)phenyl)-2-propylthiazol-5-yl)sulfonyl)carbamate,

butyl ((4-(4-((2-(tert-butyl)-1H-imidazol-1-yl)methyl)phenyl)-2-propylthiazol-5-yl)sulfonyl) carbamate,

butyl ((4′-((2-(2-hydroxypropan-2-yl)-1H-imidazol-1-yl)methyl)-5-isobutyl-[1,1′-biphenyl]-2-yl)sulfonyl)carbamate,

methyl ((4′-((2-(2-hydroxypropan-2-yl)-1H-imidazol-1-yl)methyl)-5-isobutyl-[1,1′-biphenyl]-2-yl)sulfonyl)carbamate,

butyl ((4′-((2-(tert-butyl)-1H-imidazol-1-yl)methyl)-5-propyl-[1,1′-biphenyl]-2-yl)sulfonyl)carbamate,

methyl ((4′-((2-(tert-butyl)-1H-imidazol-1-yl)methyl)-5-propyl-[1,1′-biphenyl]-2-yl)sulfonyl)carbamate,

butyl ((3-(4-((2-(tert-butyl)-1H-imidazol-1-yl)methyl)phenyl)-5-isobutylthiophen-2-yl)sulfonyl)carbamate,

methyl ((3-(4-((2-(tert-butyl)-1H-imidazol-1-yl)methyl)phenyl)-5-isobutylthiophen-2-yl)sulfonyl)carbamate,

butyl ((4-(4-((2-(2-hydroxypropan-2-yl)-1H-imidazol-1-yl)methyl)phenyl)-2-propyl thiazol-5-yl)sulfonyl)carbamate,

butyl ((4-(4-((2-isopropyl-1H-imidazol-1-yl)methyl)phenyl)-2-propylthiazol-5-yl) sulfonyl)carbamate,

butyl ((2-isobutyl-4-(4-((2-(thiazol-2-yl)-1H-imidazol-1-yl)methyl)phenyl)thiazol-5-yl) sulfonyl)carbamate,

butyl ((4-(4-((2-(2-hydroxypropan-2-yl)-1H-imidazol-1-yl)methyl)phenyl)-2-isobutyl thiazol-5-yl)sulfonyl)carbamate,

butyl ((2-isobutyl-4-(4-((2-isopropyl-1H-imidazol-1-yl)methyl)phenyl)thiazol-5-yl) sulfonyl)carbamate,

methyl ((4-(4-((2-(2-hydroxypropan-2-yl)-1H-imidazol-1-yl)methyl)phenyl)-2-isobutylthiazol-5-yl)sulfonyl)carbamate,

methyl ((2-isobutyl-4-(4-((2-isopropyl-1H-imidazol-1-yl)methyl)phenyl)thiazol-5-yl) sulfonyl)carbamate,

butyl ((2-propyl-4-(4-((2-(thiazol-2-yl)-1H-imidazol-1-yl)methyl)phenyl)thiazol-5-yl) sulfonyl)carbamate,

butyl ((4-(4-((2-(1-hydroxyethyl)-1H-imidazol-1-yl)methyl)phenyl)-2-isobutylthiazol-5-yl)sulfonyl)carbamate,

methyl ((4-(4-((2-(1-hydroxyethyl)-1H-imidazol-1-yl)methyl)phenyl)-2-isobutyl-thiazol-5-yl)sulfonyl)carbamate,

butyl ((4-(4-((2-(1-hydroxyethyl)-1H-imidazol-1-yl)methyl)phenyl)-2-propylthiazol-5-yl)sulfonyl)carbamate,

methyl ((4-(4-((2-(1-hydroxyethyl)-1H-imidazol-1-yl)methyl)phenyl)-2-propylthiazol-5-yl)sulfonyl)carbamate,

methyl ((4-(4-((2-cyclopropyl-1H-imidazol-1-yl)methyl)phenyl)-2-isobutylthiazol-5-yl)sulfonyl)carbamate,

butyl ((4-(4-((2-cyclopropyl-1H-imidazol-1-yl)methyl)phenyl)-2-isobutylthiazol-5-yl)sulfonyl)carbamate,

methyl ((4-(4-((2-cyclopropyl-1H-imidazol-1-yl)methyl)phenyl)-2-propylthiazol-5-yl)sulfonyl)carbamate,

butyl ((4-(4-((2-cyclopropyl-1H-imidazol-1-yl)methyl)phenyl)-2-propylthiazol-5-yl)sulfonyl)carbamate,

methyl ((4′-((2-(2-hydroxypropan-2-yl)-1H-imidazol-1-yl)methyl)-5-propyl-[1,1′-biphenyl]-2-yl)sulfonyl)carbamate,

butyl ((4′-((2-(2-hydroxypropan-2-yl)-1H-imidazol-1-yl)methyl)-5-propyl-[1,1′-biphenyl]-2-yl)sulfonyl)carbamate,

butyl ((4′-((2-(2-fluoropropan-2-yl)-1H-imidazol-1-yl)methyl)-5-isobutyl-[1,1′-biphenyl]-2-yl)sulfonyl)carbamate,

methyl ((5-isobutyl-4′-((2-(thiazol-2-yl)-1H-imidazol-1-yl)methyl)-[1,1′-biphenyl]-2-yl)sulfonyl)carbamate,

methyl ((4′-((2-(2-fluoropropan-2-yl)-1H-imidazol-1-yl)methyl)-5-isobutyl-[1,1′-biphenyl]-2-yl)sulfonyl)carbamate,

butyl ((5-isobutyl-4′-((2-(thiazol-2-yl)-1H-imidazol-1-yl)methyl)-[1,1′-biphenyl]-2-yl)sulfonyl)carbamate,

methyl ((5-isobutyl-4′-((2-(isopropan-2-yl)-1H-imidazol-1-yl)methyl)-[1,1′-biphenyl]-2-yl)sulfonyl)carbamate,

butyl ((5-isobutyl-4′-((2-(isopropan-2-yl)-1H-imidazol-1-yl)methyl)-[1,1′-biphenyl]-2-yl)sulfonyl)carbamate,

butyl ((5-isobutyl-4′-((2-(cyclopropan-2-yl)-1H-imidazol-1-yl)methyl)-[1,1′-biphenyl]-2-yl)sulfonyl)carbamate,

methyl ((5-isobutyl-4′-((2-(cyclopropan-2-yl)-1H-imidazol-1-yl)methyl)-[1,1′-biphenyl]-2-yl)sulfonyl)carbamate,

butyl ((5-isobutyl-3-(4-(2-(2-hydroxypropan-2-yl)-1H-imidazol-1-yl)methyl)phenyl)-2-yl)sulfonyl)carbamate,

methyl ((5-isobutyl-3-(4-(2-(2-hydroxypropan-2-yl)-1H-imidazol-1-yl)methyl)phenyl)-2-yl)sulfonyl)carbamate,

butyl ((5-isobutyl-3-(4-(2-(2-fluoropropan-2-yl)-1H-imidazol-1-yl)methyl)phenyl)-2-yl)sulfonyl)carbamate,

methyl ((5-isobutyl-3-(4-(2-(2-fluoropropan-2-yl)-1H-imidazol-1-yl)methyl)phenyl)-2-yl)sulfonyl)carbamate,

2-methoxyethyl ((5-isobutyl-3-(4-(2-(2-tertbutyl)-1H-imidazol-1-yl)methyl)phenyl)-2-yl)sulfonyl)carbamate,

butyl ((4-(4-((2-(2-fluoropropan-2-yl)-1H-imidazol-1-yl)methyl)phenyl)-2-propyl thiazol-5-yl)sulfonyl)carbamate,

methyl ((4-(4-((2-(2-fluoropropan-2-yl)-1H-imidazol-1-yl)methyl)phenyl)-2-propyl thiazol-5-yl)sulfonyl)carbamate,

butyl ((4-(4-((2-(2-fluoropropan-2-yl)-1H-imidazol-1-yl)methyl)phenyl)-2-isobutyl-thiazol-5-yl)sulfonyl)carbamate,

methyl ((4-(4-((2-(2-fluoropropan-2-yl)-1H-imidazol-1-yl)methyl)phenyl)-2-isobutyl-thiazol-5-yl)sulfonyl)carbamate,

or a pharmaceutically acceptable salt of any one of the foregoing compounds.

Further, the present provides a compound which is one or more of the following:

methyl (((4′-((2-(tert-butyl)-1H-imidazol-1-yl)methyl)-5-isobutyl-[1,1′-biphenyl]-2-yl)sulfonyl)carbamate,

methyl((4-(4-((2-(tert-butyl)-1H-imidazol-1-yl)methyl)phenyl)-2-isobutylthiazol-5-yl)sulfonyl) carbamate,

methyl ((4′-((2-(2-hydroxypropan-2-yl)-1H-imidazol-1-yl)methyl)-5-isobutyl-[1,1′-biphenyl]-2-yl)sulfonyl)carbamate,

butyl ((3-(4-((2-(tert-butyl)-1H-imidazol-1-yl)methyl)phenyl)-5-isobutylthiophen-2-yl)sulfonyl)carbamate,

methyl ((3-(4-((2-(tert-butyl)-1H-imidazol-1-yl)methyl)phenyl)-5-isobutylthiophen-2-yl)sulfonyl)carbamate,

butyl ((4-(4-((2-(2-hydroxypropan-2-yl)-1H-imidazol-1-yl)methyl)phenyl)-2-propyl thiazol-5-yl)sulfonyl)carbamate,

butyl ((4-(4-((2-isopropyl-1H-imidazol-1-yl)methyl)phenyl)-2-propylthiazol-5-yl) sulfonyl)carbamate,

butyl ((2-isobutyl-4-(4-((2-(thiazol-2-yl)-1H-imidazol-1-yl)methyl)phenyl)thiazol-5-yl) sulfonyl)carbamate,

butyl ((4-(4-((2-(2-hydroxypropan-2-yl)-1H-imidazol-1-yl)methyl)phenyl)-2-isobutyl thiazol-5-yl)sulfonyl)carbamate,

butyl ((2-isobutyl-4-(4-((2-isopropyl-1H-imidazol-1-yl)methyl)phenyl)thiazol-5-yl) sulfonyl)carbamate,

methyl ((4-(4-((2-(2-hydroxypropan-2-yl)-1H-imidazol-1-yl)methyl)phenyl)-2-isobutylthiazol-5-yl)sulfonyl)carbamate,

methyl ((2-isobutyl-4-(4-((2-isopropyl-1H-imidazol-1-yl)methyl)phenyl)thiazol-5-yl) sulfonyl)carbamate,

butyl ((2-propyl-4-(4-((2-(thiazol-2-yl)-1H-imidazol-1-yl)methyl)phenyl)thiazol-5-yl) sulfonyl)carbamate,

butyl ((4-(4-((2-(1-hydroxyethyl)-1H-imidazol-1-yl)methyl)phenyl)-2-isobutylthiazol-5-yl)sulfonyl)carbamate,

methyl ((4-(4-((2-(1-hydroxyethyl)-1H-imidazol-1-yl)methyl)phenyl)-2-isobutyl-thiazol-5-yl)sulfonyl)carbamate,

butyl ((4-(4-((2-(1-hydroxyethyl)-1H-imidazol-1-yl)methyl)phenyl)-2-propylthiazol-5-yl)sulfonyl)carbamate,
methyl ((4-(4-((2-(1-hydroxyethyl)-1H-imidazol-1-yl)methyl)phenyl)-2-propylthiazol-5-yl)sulfonyl)carbamate,

methyl ((4-(4-((2-cyclopropyl-1H-imidazol-1-yl)methyl)phenyl)-2-isobutylthiazol-5-yl)sulfonyl)carbamate,

butyl ((4-(4-((2-cyclopropyl-1H-imidazol-1-yl)methyl)phenyl)-2-isobutylthiazol-5-yl)sulfonyl)carbamate,

methyl ((4-(4-((2-cyclopropyl-1H-imidazol-1-yl)methyl)phenyl)-2-propylthiazol-5-yl)sulfonyl)carbamate,

butyl ((4-(4-((2-cyclopropyl-1H-imidazol-1-yl)methyl)phenyl)-2-propylthiazol-5-yl)sulfonyl)carbamate,

methyl ((4′-((2-(2-hydroxypropan-2-yl)-1H-imidazol-1-yl)methyl)-5-propyl-[1,1′-biphenyl]-2-yl)sulfonyl)carbamate,

butyl ((4′-((2-(2-hydroxypropan-2-yl)-1H-imidazol-1-yl)methyl)-5-propyl-[1,1′-biphenyl]-2-yl)sulfonyl)carbamate,

butyl ((4′-((2-(2-fluoropropan-2-yl)-1H-imidazol-1-yl)methyl)-5-isobutyl-[1,1′-biphenyl]-2-yl)sulfonyl)carbamate,

methyl ((5-isobutyl-4′-((2-(thiazol-2-yl)-1H-imidazol-1-yl)methyl)-[1,1′-biphenyl]-2-yl)sulfonyl)carbamate,

methyl ((4′-((2-(2-fluoropropan-2-yl)-1H-imidazol-1-yl)methyl)-5-isobutyl-[1,1′-biphenyl]-2-yl)sulfonyl)carbamate,

butyl ((5-isobutyl-4′-((2-(thiazol-2-yl)-1H-imidazol-1-yl)methyl)-[1,1′-biphenyl]-2-yl)sulfonyl)carbamate,

methyl ((5-isobutyl-4′-((2-(isopropan-2-yl)-1H-imidazol-1-yl)methyl)-[1,1′-biphenyl]-2-yl)sulfonyl)carbamate,

butyl ((5-isobutyl-4′-((2-(isopropan-2-yl)-1H-imidazol-1-yl)methyl)-[1,1′-biphenyl]-2-yl)sulfonyl)carbamate,

butyl ((5-isobutyl-4′-((2-(cyclopropan-2-yl)-1H-imidazol-1-yl)methyl)-[1,1′-biphenyl]-2-yl)sulfonyl)carbamate,

methyl ((5-isobutyl-4′-((2-(cyclopropan-2-yl)-1H-imidazol-1-yl)methyl)-[1,1′-biphenyl]-2-yl)sulfonyl)carbamate,

butyl ((5-isobutyl-3-(4-(2-(2-hydroxypropan-2-yl)-1H-imidazol-1-yl)methyl)phenyl)-2-yl)sulfonyl)carbamate,

methyl ((5-isobutyl-3-(4-(2-(2-hydroxypropan-2-yl)-1H-imidazol-1-yl)methyl)phenyl)-2-yl)sulfonyl)carbamate,

butyl ((5-isobutyl-3-(4-(2-(2-fluoropropan-2-yl)-1H-imidazol-1-yl)methyl)phenyl) yl)sulfonyl)carbamate,

methyl ((5-isobutyl-3-(4-(2-(2-fluoropropan-2-yl)-1H-imidazol-1-yl)methyl)phenyl) yl)sulfonyl)carbamate,

2-methoxyethyl ((5-isobutyl-3-(4-(2-(2-tertbutyl)-1H-imidazol-1-yl)methyl)phenyl)-2-yl)sulfonyl)carbamate,

butyl ((4-(4-((2-(2-fluoropropan-2-yl)-1H-imidazol-1-yl)methyl)phenyl)-2-propyl thiazol-5-yl)sulfonyl)carbamate,

methyl ((4-(4-((2-(2-fluoropropan-2-yl)-1H-imidazol-1-yl)methyl)phenyl)-2-propyl thiazol-5-yl)sulfonyl)carbamate,

butyl ((4-(4-((2-(2-fluoropropan-2-yl)-1H-imidazol-1-yl)methyl)phenyl)-2-isobutylthiazol-5-yl)sulfonyl)carbamate,

methyl ((4-(4-((2-(2-fluoropropan-2-yl)-1H-imidazol-1-yl)methyl)phenyl)-2-isobutylthiazol-5-yl)sulfonyl)carbamate,

butyl ((4′-((2-(tert-butyl)-1H-imidazol-1-yl)methyl)-3′-fluoro-5-isobutyl-[1,1′-biphenyl]-2-yl)sulfonyl)carbamate,

methyl (4′-((2-(tert-butyl)-1H-imidazol-1-yl)methyl)-3′-fluoro-5-isobutyl-[1,1′-biphenyl]-2-yl)sulfonylcarbamate,

or a pharmaceutically acceptable salt of any one of the foregoing compounds.

In any event, preferred compounds of the invention include the compounds of the examples described hereinafter.

Pharmaceutical Formulations and Medical Uses

The present disclosure also provides a pharmaceutical composition comprising a therapeutically acceptable amount of a compound of Formula I as described herein or a therapeutically acceptable salt thereof, together with at least one pharmaceutically acceptable carrier, excipient or diluent.

The compounds of the invention are indicated both in the therapeutic, palliative, and/or diagnostic treatment, as well as the prophylactic treatment (by which we include preventing and/or abrogating deterioration and/or worsening of a condition) of any of the above conditions.

Compounds of the invention will normally be administered orally, intravenously, subcutaneously, buccally, rectally, dermally, nasally, tracheally, bronchially, by any other parenteral route, or via inhalation or pulmonary route, or any combination thereof, in a pharmaceutically acceptable dosage form, in solution, in suspension, in emulsion, including nanosuspensions, or in liposome formulation. Additional methods of administration include, but are not limited to, intraarterial, intramuscular, intraperitoneal, intraportal, intradermal, epidural, intrathecal administration, or any combination thereof.

The pharmaceutical composition may be in the form of an oral dosage form such a lozenge, capsule or a syrup.

In some embodiments, the compounds of the invention may be administered alone (e.g. separately), and/or sequentially, and/or in parallel at the same time (e.g. concurrently), using different administrative routes, but are preferably administered by way of known pharmaceutical formulations, including tablets, capsules or elixirs for oral administration, suppositories for rectal administration, sterile solutions, suspensions or emulsions for parenteral or intramuscular administration, or via inhalation, and the like. Administration through inhalation is preferably done by using a nebulizer, thus delivering the compound of the invention to the small lung tissue including the alveoli and bronchioles, preferably without causing irritation or cough in the treated subject.

Administration of a therapeutically effective amount of a compound of the invention may be performed by a combination of administrative routes, either separately (e.g. about 2 or more hours apart from one another), sequentially (e.g. within about 2 hours of one another), or in parallel at the same time (e.g. concurrently), including via inhalation and orally, achieving an effective dosage.

As used herein, the expression “therapeutically effective amount” means an amount of a compound as described herein that is sufficient to induce a desired therapeutic effect in a patient to which the compound is administered. Further, a patient as described herein may be a human.

There is also provided a compound as described herein, or a pharmaceutically acceptable salt thereof, or a pharmaceutical composition as described herein, for use as a medicament. The medicament may be a medicament for the treatment and/or prevention of a disease, disorder and/or condition associated with the peptide angiotensin II. The medicament may be used in the treatment and/or prevention of a disease, disorder and/or condition associated with the peptide angiotensin II wherein said medicament exhibits an acceptable level of CYP inhibition of one or more CYPs.

Thus, there is provided a compound as described herein, or a pharmaceutically acceptable salt thereof, or a pharmaceutical composition as described herein, for use in the treatment and/or prevention of a disease, disorder and/or condition associated with the peptide angiotensin II. Compounds described herein, and pharmaceutically acceptable salts thereof, optionally in the form of a pharmaceutical composition, may thus be used in the treatment and/or prevention of a disease, disorder and/or condition associated with the peptide angiotensin II wherein said compound, pharmaceutically acceptable salt thereof, or pharmaceutical composition, exhibits an acceptable level of CYP inhibition of one or more CYPs.

Further, there is provided a use of a compound as described herein, or a pharmaceutically acceptable salt thereof, or a pharmaceutical composition as described herein, for use in the manufacture of a medicament for the treatment and/or prevention of a disease, disorder and/or condition associated with the peptide angiotensin II.

Moreover, there is provided a method for treatment and/or prevention of a disease, disorder and/or condition associated with the peptide angiotensin II which method comprises the step of administering a therapeutically effective amount of a compound of Formula I as described herein, or a pharmaceutically acceptable salt thereof, or a pharmaceutical composition as described herein to a patient in need thereof.

For the avoidance of doubt, the expression “exhibits an acceptable level of CYP inhibition” means that CYP inhibition is not desired and/or wherein the level of CYP inhibition is, for example, less than 50%, such as between 40 to 20%, for example less than 20%, such as between 10 to 0%, for example less than 0%, such as −50%.

For the avoidance of doubt, the expression “disease, disorder and/or condition associated with the peptide angiotensin II” means a disease, disorder and/or condition in which activation of AT2 receptors is desired or required.

Thus, in some embodiments, there is provided a method of treating a disease, disorder and/or condition in which activation of AT2 receptors is desired or required but in which inhibition of CYPs is not desired, which method comprises administering a therapeutically effective amount of a compound of the invention either separately, sequentially, or in parallel at the same time, preferably via inhalation and orally, in order to achieve effective amount or dosage, to a patient in need of such a therapy. Such formulations may be prepared in accordance with standard and/or accepted pharmaceutical practice.

Subjects suitable to be treated with formulations of the present invention include, but are not limited to, mammalian subjects, in particular human subjects.

The compounds as described herein, or a pharmaceutically acceptable salt thereof, are expected to be useful in those diseases, disorders and/or conditions where AT2 receptors are expressed and their stimulation is desired or required. In particular, the compounds as described herein, or a pharmaceutically acceptable salt thereof, are useful in the treatment of a disease, disorder and/or condition in which activation of AT2 receptors is desired or required but in which inhibition of CYPs is not desired.

By a “disease, disorder and/or condition associated with angiotensin II”, we include diseases, disorders and/or conditions that are known to be treatable by activation of AT2 receptors, such as those mentioned hereinafter, but wherein existing treatments of such conditions may comprise administration of other therapeutic agents that are metabolized by CYPs. Such diseases, disorders and/or conditions may thus include conditions in which inhibition of at least one CYP enzyme is not required, advantageous and/or desirable, or in which such inhibition is or would be detrimental to the patient.

As described herein, the compounds of the invention are useful because they possess pharmacological activity, and/or are metabolised in the body following oral or parenteral administration to form compounds that possess pharmacological activity.

In particular, compounds of the invention are agonists of Ang II receptors. Compounds of the invention are thus expected to be useful in those diseases, disorders and/or conditions in which endogenous production of Ang II is deficient and/or where an increase in the activity of Ang II receptors is desired or required.

More particularly, compounds of the invention are agonists of the AT2 receptor, and, especially, are selective (vs. the AT1 receptor) agonists of that sub-receptor, for example as may be demonstrated in the tests described below.

AT2 receptor agonists include those that fully, and those that partially, activate the AT2 receptor. Compounds of the invention may thus bind selectively to the AT2 receptor, and exhibit agonist activity at the AT2 receptor. By compounds that “bind selectively” to the AT2 receptor, we include that the affinity ratio for the relevant compound (AT2:AT1) at a given concentration is at least 50:1, such as at least 100:1, preferably at least 1000:1.

In this respect, compounds of the invention are indicated in the treatment of conditions characterized by vasoconstriction, fibrosis, increased cell growth and/or differentiation, increased cardiac contractility, increased cardiovascular hypertrophy, and/or increased fluid and electrolyte retention, as well as skin disorders and musculoskeletal disorders.

Compounds of the invention may also exhibit thromboxane receptor activity. In this respect, compounds of the invention may have an inhibitory effect on platelet activation and/or aggregation (and thus e.g. an antithrombotic effect), and/or may reduce vasoconstriction and/or bronchoconstriction in a therapeutic manner.

Compounds of the invention are further indicated in the treatment of stress-related disorders, and/or in the improvement of microcirculation and/or mucosa-protective mechanisms.

Thus, compounds of the invention are expected to be useful in the treatment of disorders, which may be characterised as indicated above, and which are of, for example, the gastrointestinal tract, the cardiovascular system, the respiratory tract, the kidneys, the eyes, the female reproductive (ovulation) system and the central nervous system (CNS).

Disorders of the gastrointestinal tract that may be mentioned include oesophagitis, Barrett's oesophagus, gastric ulcers, duodenal ulcers, dyspepsia (including non-ulcer dyspepsia), gastro-oesophageal reflux, irritable bowel syndrome (IBS), inflammatory bowel disease (IBD), pancreatitis, hepatic disorders (such as hepatitis), gall bladder disease, multiple organ failure (MOF) and sepsis. Other gastrointestinal disorders that may be mentioned include xerostomia, gastritis, gastroparesis, hyperacidity, disorders of the bilary tract, coelicia, Crohn's disease, ulcerative colitis, diarrhoea, constipation, colic, dysphagia, vomiting, nausea, indigestion and Sjogren's syndrome.

Disorders of the respiratory tract that may be mentioned include inflammatory disorders, such as asthma, obstructive lung diseases (such as chronic obstructive lung disease), pneumonitis, pulmonary hypertension, and adult respiratory distress syndrome.

Disorders of the kidneys that may be mentioned include renal failure, nephritis and renal hypertension.

Disorders of the eyes that may be mentioned include diabetic retinopathy, premature retinopathy and retinal microvascularisation.

Disorders of the female reproductive system that may be mentioned include ovulatory dysfunction.

Cardiovascular disorders that may be mentioned include hypertension, cardiac hypertrophy, cardiac failure (including heart failure with preserved ejection fraction), artherosclerosis, arterial thrombosis, venous thrombosis, endothelial dysfunction, endothelial lesions, post-balloon dilatation stenosis, angiogenesis, diabetic complications, microvascular dysfunction, angina, cardiac arrhythmias, claudication intermittens, preeclampsia, myocardial infarction, reinfarction, ischaemic lesions, erectile dysfunction and neointima proliferation.

Disorders of the CNS that may be mentioned include cognitive dysfunctions, dysfunctions of food intake (hunger/satiety) and thirst, stroke, cerebral bleeding, cerebral embolus and cerebral infarction, multiple sclerosis (MS), Alzheimer's disease and Parkinson's disease.

Compounds of the invention may also be useful in the modulation of growth metabolism and proliferation, for example in the treatment of ageing, hypertrophic disorders, prostate hyperplasia, autoimmune disorders (e.g. arthritis, such as rheumatoid arthritis, or systemic lupus erythematosus), psoriasis, obesity, neuronal regeneration, the healing of ulcers, inhibition of adipose tissue hyperplasia, stem cell differentiation and proliferation, fibrotic disorders, cancer (e.g. in, or of, the gastrointestinal tract (including the oesophagus or the stomach), the prostate, the breast, the liver, the kidneys, as well as lymphatic cancer, lung cancer, ovarian cancer, pancreatic cancer, hematologic malignacies, etc.), apoptosis, tumours (generally) and hypertrophy, diabetes, neuronal lesions and organ rejection.

Compounds of the invention are also useful in the treatment of stroke, spinal cord injury, sickle cell disease, muscular dystrophy, cancer treatment-related cardiotoxicity, peripheral neuropathy and, in particular, systemic sclerosis.

The disease, disorder and/or condition associated with the peptide angiotensin II described herein may be selected from the group consisting of hypertension, heart failure, stroke, nephropathy, pulmonary fibrosis such as idiopathic pulmonary fibrosis, sclerosis such as systemic sclerosis, sarcoidosis such as pulmonary sarcoidosis and any combination thereof.

Particular diseases, disorders and/or conditions in which activation of AT2 receptors is desired or required but in which inhibition of CYP enzymes is not desired are interstitial lung diseases (e.g. pulmonary fibrosis, IPF, systemic sclerosis and sarcoidosis), autoimmune diseases (e.g. rheumatoid arthritis, systemic lupus erythematosus, multiple sclerosis, psoriasis and inflammatory bowel disease), chronic kidney diseases (e.g. diabetic nephropathy), pulmonary hypertension, pulmonary arterial hypertension and/or infarction (e.g. myocardial infarction and stroke). Thus, compounds of the invention are particularly useful in the treatment of interstitial lung diseases, such as IPF; autoimmune diseases, such as rheumatoid arthritis; chronic kidney diseases, such as diabetic nephropathy; pulmonary hypertension, including pulmonary arterial hypertension; and/or infarction, such as myocardial infarction.

Compounds of the invention are particularly indicated in the treatment and/or prevention of ILDs, such as sarcoidosis or fibrosis, more specifically pulmonary fibrosis and particularly IPF, as well as conditions that may trigger ILDs, such as systemic sclerosis, rheumatoid arthritis, myositis or systemic lupus erythematosus, or are otherwise associated with ILDs, such as pulmonary hypertension and/or pulmonary arterial hypertension.

Compounds of the invention are particularly useful in the treatment of pulmonary fibrosis, in particular IPF.

According to a further aspect of the present invention, there is provided a method of treatment of pulmonary fibrosis, and in particular IPF, which method comprises administration of a therapeutically effective amount of a compound of the invention to a person suffering from such a condition.

In the treatment of pulmonary fibrosis, including IPF, compounds of the invention may have an anti-fibrotic effect, with reduction of fibrosis and prevention of further deposition of extracellular matrix. Compounds of the invention may reduce lung scarring/wound healing and also have an anti-apoptotic effect, thereby preventing apoptosis of alveolar endothelial cells, being an initiating factor for the development of pulmonary fibrosis. Compounds of the invention may also have an anti-proliferative effect, thus reducing the cancer-like proliferation of fibroblasts and myofibroblasts in pulmonary fibrosis. Compounds of the invention may also improve vascular remodelling in pulmonary fibrosis, thereby reducing secondary pulmonary hypertension.

Compounds of the invention may further demonstrate antiinflammatory, anti-growth factor (e.g. transforming growth factor beta) and/or anti-cytokine effects.

In addition, compounds of the invention may also be useful in the treatment or prevention of any fibrotic condition of one or more internal organs characterised by the excessive accumulation of fibrous connective tissue, and/or in the treatment or prevention of fibrogenesis and the morbidity and mortality that may be associated therewith. Such fibrosis may be associated with an acute inflammatory condition, such as acute respiratory distress syndrome (ARDS), severe acute respiratory syndrome (SARS), and multiple-organ inflammation, injury and/or failure, which may be caused by internal or external trauma (e.g. injury), or by an infection.

Such conditions may thus result from sepsis or septic shock caused by a viral, bacterial or fungal infection. Furthermore, acute lung injury, ARDS and, particularly, SARS may be caused by viruses, such as coronaviruses, include the novel SARS coronavirus 2 (SARS-CoV-2), which may result in internal tissue damage, dysfunction of relevant internal (e.g. mucosal) tissues, such as the respiratory epithelium, and so result in virally-induced pneumonia, impaired lung function, respiratory dysfunction, distress and/or failure. Such tissue damage may also give rise to severe fibrosis. Such tissue damage may also give rise to severe fibrosis. For example, the SARS disease caused by the novel coronavirus SARS-CoV-2 (coronavirus disease 2019 or COVID-19) is known in many cases to result in fibrosis.

Compounds of the invention have the advantage that they are more potent than, and/or are stable to metabolic hydrolysis, and/or have acceptable level of CYP enzyme inhibition as mentioned hereinbefore.

The compounds of the invention may also have the advantage that they may be more efficacious than, be less toxic than, be longer acting than, be more potent than, produce fewer side effects than, be more easily absorbed than, and/or have a better pharmacokinetic profile (e.g. higher oral bioavailability and/or lower clearance) than, and/or have other useful pharmacological, physical, or chemical properties than compounds known in the prior art, whether for use in the treatment of any of the disease states mentioned above (such as IPF) or otherwise. Such effects may be evaluated clinically, objectively and/or subjectively by a health care professional, a treatment subject or an observer.

Combinations

The compounds of the present disclosure, or a pharmaceutically acceptable salt thereof, may be used in combination with one or more further pharmaceutical drugs in combination therapy to treat the various conditions, including those mentioned hereinbefore. Because compounds of the invention exhibit minimal CYP enzyme inhibition, such combinations are particularly advantageous when the other therapeutic agents that are employed for use in the relevant condition are themselves metabolized by CYP enzymes.

Thus, compounds of the invention may be administered in combination with other AT2 agonists that are known in the art, such as C21, as well as in combination with AT1 receptor antagonists that are known in the art, and/or in combination with an inhibitor of angiotensin converting enzyme (ACE).

Non-limiting but illustrative examples of AT1 receptor antagonists that can be used according to the embodiments include azilsartan, candesartan, eprosartan, fimasartan, irbesartan, losartan, milfasartan, olmesartan, pomisartan, pratosartan, ripiasartan, saprisartan, tasosartan, telmisartan, valsartan and/or combinations thereof. Non-limiting but illustrative examples of ACE inhibitors that can be used according to the embodiments include captopril, zofenopril, enalapril, ramipril, quinapril, perindopril, lisinopril, benazepril, imidapril, trandolapril, fosinopril, moexipril, cilazapril, spirapril, temocapril, alacepril, ceronapril, delepril, moveltipril, and/or combinations thereof.

Other active ingredients that may be administered in combination with compounds of the invention include disodium cromoglycate; endothelin receptor antagonists, such as bosentan, ambrisentan, sitaxentan and macitentan; PDE5 inhibitors, such as sildenafil and tadalafil; prostacyclin (epoprostenol) and analogues thereof, such as iloprost and treprostinil; other biologics including interferon gamma-lb, etanercept, infliximab and adalimumab; and methotrexate. Further active ingredients in development that may be co-administered with compounds of the invention include pamrevlumab (anti-CTGF, Fibrogen); GLPG1690 (autotaxin inhibitor, Galapagos), TD139 (Galectin-3 inhibitor, Galecto), PRM-151 (recombinant pentraxin-2, Promedior), BBT-877 (autotaxin inhibitor, Boehringer/Bridge), CC-90001 (JNK inhibitor, Celgene), PBI-4050 (dual GPR40 agonist/GPR84 antagonist, Prometic), BMS-986020 (lysophosphatidic acid receptor antagonist, BMS), RVT-1601 (mast cell stabilizer, Respivant), SMO4646 (wntsignal inhibitor, United Therapeutics), KD25 (Rho associated kinase inhibitor, Kadmon Holdings), BG00011 (integrin antagonist, Biogen), PLN-74809 (integrin antagonist, Pilant Therapeutics), Saracatinib (src kinase inhibitor, AstraZeneca), PAT-1251 (lysyloxidase inhibitor 2, PharmAkea), ABM-125 (IL-25 MAB, Abeome) and TA5-115 (multi-kinase inhibitor, Otsuka).

When the condition to be treated is an interstitial lung disease, such as IPF, systemic sclerosis or fibrotic diseases that are known in the art, compounds of the invention are preferably administered in combination with a Galectin-3 inhibitor, a lysophosphatidic acid receptor 1 (LPA1) antagonist, an autotaxin (ATX) inhibitor, a recombinant human pentraxin-2 protein or established therapies for such treatment, including but not limited to pirfenidone and/or nintedanib. Preferably, the combination of compound of the invention is with pirfenidone, or a pharmaceutically-acceptable salt thereof, which compound is known to be metabolized by CYP enzymes, such as CYP1A.

Further, when the condition to be treated is a chronic kidney related disease, compounds of the invention are preferably administered in combination with one or more other drugs that are also used in such treatments, such as irbesartan and/or torsemide, which compounds are known to be metabolized by CYP enzymes, such as CYP2C9.

When the condition to be treated is pulmonary hypertension, compounds of the invention are preferably administered in combination with one or more other drugs that are also used in such treatment, such as selexipag and/or sildenafil, which compounds are known to be metabolized by CYP enzymes, such as CYP3A4.

When the condition to be treated or prevented is myocardial infarction and/or a stroke related disease, compounds of the invention are preferably administered in combination with one or more other drugs that are also used in such treatment, such as propranolol, warfarin, clopidogrel, atorvastatin, cilostazol, lidocaine and/or simvastatin, or a pharmaceutically-acceptable salt thereof, which compounds are known to be metabolized by CYP enzymes, such as CYP1A, CYP2CP and/or CYP3A4.

When the condition to be treated is an autoimmune disease, such as rheumatoid arthritis, multiple sclerosis or psoriasis, compounds of the invention are preferably administered in combination with one or more other drugs that are also used in such treatment, including but not limited to non-steroidal anti-inflammatory drugs (NSAIDs), such as naproxen, celecoxib, meloxicam or an analogue thereof (e.g. piroxicam) orindomethacin; or a drug such as tizanidine, cyclophosphamide, cyclosporine, deflazacort and/or hydrocortisone, riluzole, or a pharmaceutically acceptable salt thereof, which compounds are known to be metabolized by CYP enzymes, such as CYP1A, CYP2CP, CYP2C19 and/or CYP3A4.

Thus, compounds of the invention are particularly useful in the treatment of a disease, disorder and/or condition in which activation of the AT2 receptor is desired or required but in which inhibition of CYP enzymes is not desired and so may be administered to treat diseases, including those mentioned hereinbefore, in combination with one or more of the other therapeutic agents mentioned hereinbefore, which are metabolized through a CYP enzyme pathway, is or may be useful, including pirfenidone, naproxen, propranolol, riluzole, tizanidine, warfarin, celecoxib, clopidogrel, irbesartan, meloxicam, piroxicam, torsemide, cyclophosphamide, indomethacin, atorvastatin, cilostazol, cyclosporine, deflazacort, hydrocortisone, lidocaine, selexipag, sildenafil and/or simvastatin. Most preferably, the compounds of the invention are administered in combination with pirfenidone to treat an interstitial lung disease, such as IPF.

Therapeutic agents that may be used in conjunction with compounds of the invention include variously-applied standard treatments for viral infections, including antibody therapies (e.g. LY-CoV555/LY-CoV016 (bamlanivimab and etesevimab), LY-CoV555 (bamlanivimab, Eli Lilly), REGN-COV2 (casirivimab and imdevimab), REGN3048-3051, TZLS-501, SNG001 (Synairgen), eculizumab (Soliris; Alexion Pharmaceuticals), ravulizumab (Ultomiris; Alexion Pharmaceuticals), lenzilumab, leronlimab, tocilizumab (Actemra; Roche), sarilumab (Kevzara; Regeneron Pharma), and Octagam (Octapharma)), including antiviral medicines (e.g. oseltamivir, remdesivir, favilavir, molnupiravir, simeprevir, daclatasvir, sofosbuvir, ribavirin, umifenovir, lopinavir, ritonavir, lopinavir/ritonavir (Kaletra; AbbVie Deutschland GmbH Co. KG), teicoplanin, baricitinib (Olumiant; Eli Lilly), ruxolitinib (Jakavi; Novartis), tofacitinib (Xeljanz; Pfizer), the TMPRSS2 inhibitor camostat, or camostat mesylate, Actemra (Roche), AT-100 (rhSP-D), MK-7110 (CD24Fc; Merck)), OYA1 (OyaGen9), BPI-002 (BeyondSpring), NP-120 (Ifenprodil; Algernon Pharmaceuticals), and Galidesivir (Biocryst Pharma), antiinflammatory agents (e.g. NSAIDs, such as ibuprofen, ketorolac, naproxen, and the like), chloroquine, hydroxychloroquine, interferons (e.g. interferon beta (interferon beta-1a), tocilizumab (Actemra), lenalidomide, pomalidomide and thalidomide), analgesics (e.g. paracetamol or opioids), antitussive agents (e.g. dextromethorphan), vaccinations (e.g. INO-4800 by Inovio Pharmaceuticals and Beijing Advaccine Biotechnology, if available), COVID-19 convalescent plasma (CCP) and/or passive antibody therapy with antibodies from blood of people who have recovered from infection with SARS-CoV or SARS-CoV-2.

Further therapeutic agents that may be mentioned include anti-fibrotics (e.g. nintedanib and, particularly, pirfenidone), vitamins (e.g. vitamin B, C and D) and mucolytics such as acetylcysteine and ambroxol.

Other therapeutic agents that may be used in conjunction with compounds of the invention or pharmaceutically acceptable salts thereof in accordance with the invention include corticosteroids. Corticosteroids include both naturally-occurring corticosteroids and synthetic corticosteroids.

Naturally-occurring corticosteroids that may be mentioned include cortisol (hydrocortisone), aldosterone, corticosterone, cortisone, pregnenolone, progesterone, as well as naturally-occurring precursors and intermediates in corticosteroid biosynthesis, and other derivatives of naturally-occurring corticosteroids, such as 11-deoxycortisol, 21-deoxycortisol, 11-dehydrocorticosterone, 11-deoxycorticosterone, 18-hydroxy-11-deoxycorticosterone, 18-hydroxycorticosterone, 21-deoxycortisone, 11β-hydroxypregnenolone, 11β,17α,21-trihydroxypregnenolone, 17α,21-dihydroxypregnenolone, 17α-hydroxypregnenolone, 21-hydroxypregnenolone, 11-ketoprogesterone, 11β-hydroxyprogesterone, 17α-hydroxyprogesterone and 18-hydroxyprogesterone.

Synthetic corticosteroids that may be mentioned include those of the hydrocortisone-type (Group A), such as cortisone acetate, hydrocortisone aceponate, hydrocortisone acetate, hydrocortisone buteprate, hydrocortisone butyrate, hydrocortisone valerate, tixocortol and tixocortol pivalate, prednisolone, methylprednisolone, prednisone, chloroprednisone, cloprednol, difluprednate, fludrocortisone, fluocinolone, fluperolone, fluprednisolone, loteprednol, prednicarbate and triamcinolone; acetonides and related substances (Group B), such as amcinonide, budesonide, desonide, fluocinolone cetonide, fluocinonide, halcinonide, triamcinolone acetonide, ciclesonide, deflazacort, formocortal, fludroxycortide, flunisolide and fluocinolone acetonide, those of the (beta)methasone-type (Group C), such as beclomethasone, betamethasone, beta methasone dipropionate and beta methasone valerate, dexamethasone, fluocortolone, halometasone, mometasone and mometasone furoate, alclometasone and alclometasone dipropionate, clobetasol and clobetasol propionate, clobetasone and clobetasone butyrate, clocortolone, desoximetasone, diflorasone, difluocortolone, fluclorolone, flumetasone, fluocortin, fluprednidene and fluprednidene acetate, fluticasone, fluticasone furoate and fluticasone propionate, meprednisone, paramethasone, prednylidene, rimexolone and ulobetasol; those of the progesterone-type, such as flugestone, fluorometholone, medrysone and prebediolone acetate, and progesterone derivatives (progestins), such as chlormadinone acetate, cyproterone acetate, medrogestone, medroxyprogesterone acetate, megestrol acetate and segesterone acetate; as well as other corticosteroids, such as cortivazol and 6-methyl-11β,17β-dihydroxy-17α-(1-propynyl)androsta-1,4,6-trien-3-one.

Preferred corticosteroids include cortisone, prednisone, prednisolone, methylprednisolone and, especially, dexamethasone.

Further, therapeutic agents that may be used in conjunction with compounds of the invention or pharmaceutically acceptable salts thereof include H2 receptor blockers, anticoagulants, anti-platelet drugs, as well as statins, antimicrobial agents and anti-allergic/anti-asthmatic drugs.

H2 receptor blockers that may be mentioned include famotidine. Anticoagulants that may be mentioned include heparin and low-molecular-weight heparins (e.g. bemiparin, nadroparin, reviparin, enoxaparin, parnaparin, certoparin, dalteparin, tinzaparin);

directly acting oral anticoagulants (e.g. dabigatran, argatroban, rivaroxaban, apixaban, edoxaban, betrixaban, darexaban, otamixaban, letaxaban, eribaxaban, hirudin, lepirudin and bivalirudin); coumarin type vitamin K antagonists (e.g. coumarin, acenocoumarol, phenprocoumon, atromentin and phenindione) and synthetic pentasaccharide inhibitors of factor Xa (e.g. fondaparinux, idraparinux and idrabiotaparinux). Anti-platelet drugs that may be mentioned include irreversible cyclooxygenase inhibitors (e.g. aspirin and triflusal); adenosine diphosphate receptor inhibitors (e.g. cangrelor, clopidogrel, prasugrel, ticagrelor and ticlopidine); phosphodiesterase inhibitors (e.g. cilostazol); protease-activated receptor-1 antagonists (e.g. vorapaxar); glycoprotein IIB/IIIA inhibitors (e.g. abciximab, eptifibatide and tirofiban); adenosine reuptake inhibitors (e.g. dipyridamole); and thromboxane inhibitors (e.g. terutroban, ramatroban, seratrodast and picotamide).

Statins that may be mentioned include atorvastatin, simvastatin and rosuvastatin. Antimicrobial agents that may be mentioned include azithromycin, ceftriaxone, cefuroxime, doxycycline, fluconazole, piperacillin, tazobactam and teicoplanin. Anti-allergic/anti-asthmatic drugs that may be mentioned include chlorphenamine, levocetirizine and montelukast.

Subjects may thus also (and/or may be already) be receiving one or more of any of the other therapeutic agents mentioned above, by which we mean receiving a prescribed dose of one or more of those other therapeutic agents, prior to, in addition to, and/or following, treatment with compounds of the invention or pharmaceutically acceptable salts thereof.

When compounds of the invention are “combined” with other therapeutic agents as mentioned hereinbefore, the active ingredients may be administered together in the same formulation, or administered separately (simultaneously or sequentially) in different formulations.

Such combination products provide for the administration of compounds of the invention in conjunction with the other therapeutic agent, and may thus be presented either as separate formulations, wherein at least one of those formulations comprises a compound of the invention, and at least one comprises the other therapeutic agent, or may be presented (i.e. formulated) as a combined preparation (i.e. presented as a single formulation including a compound of the invention and the other therapeutic agent).

The combination may be provided as a kit of parts optionally including instructions for use. Alternatively, the combination may be provided as a single composition or formulation, wherein at least one of those compositions or formulations comprises a compound of the invention, and at least one comprises the other therapeutic agent, or may be presented (i.e. formulated) as a combined preparation (i.e. presented as a single composition or formulation including a compound of the invention and the other therapeutic agent).

Thus, there is further provided:

(1) a pharmaceutical composition or formulation including a compound of the invention; a therapeutic agent that is known to be metabolized by a CYP enzyme, such as any of those mentioned hereinbefore; and a pharmaceutically-acceptable excipient (e.g. adjuvant, diluent or carrier), which composition or formulation is hereinafter referred to as a “combined preparation”; and

(2) a kit of parts comprising components:

(A) a pharmaceutical composition or formulation including a compound of the invention in admixture with a pharmaceutically-acceptable adjuvant, diluent or carrier; and

(B) a pharmaceutical composition or formulation including a therapeutic agent that is known to be metabolized by a CYP enzyme, such as any of those mentioned hereinbefore, in admixture with a pharmaceutically-acceptable adjuvant, diluent or carrier, which components (A) and (B) are each provided in a form that is suitable for administration in conjunction with the other.

In a further aspect of the invention, there is provided a process for the preparation of a combined preparation as hereinbefore defined, which process comprises bringing into association a compound of the invention, the other therapeutic agent, and at least one (e.g. pharmaceutically-acceptable) excipient.

In a further aspect of the invention, there is provided a process for the preparation of a kit-of-parts as hereinbefore defined, which process comprises bringing into association components (A) and (B). As used herein, references to bringing into association will mean that the two components are rendered suitable for administration in conjunction with each other.

Thus, in relation to the process for the preparation of a kit of parts as hereinbefore defined, by bringing the two components “into association with” each other, we include that the two components of the kit of parts may be:
(i) provided as separate compositions or formulations (i.e. independently of one another), which are subsequently brought together for use in conjunction with each other in combination therapy; or
(ii) packaged and presented together as separate components of a “combination pack” for use in conjunction with each other in combination therapy.

Thus, there is further provided a kit of parts comprising:
(I) one of components (A) and (B) as defined herein; together with
(II) instructions to use that component in conjunction with the other of the two components.

Depending upon the patient to be treated and the route of administration, the compounds of the invention may be administered at varying doses. Although doses will vary from patient to patient, suitable daily doses are in the range of about 0.1 to about 1000 mg (e.g. 0.1, 0.5, 1, 2, 5, 10, 15, 20, 25, 30, 35, 40, 45, 50, 75, 100, 150, 200, 250, 300, 350, 400, 450, 500, 550, 600, 650, 700, 750, 800, 850, 900, 950, 1000 mg, and the like, or any range or value therein) per patient, administered in single or multiple doses. More preferred daily doses are in the range of about 0.1 to about 250 mg (e.g., 0.2, 0.3, 0.4, 0.5, 1, 1.5, 2, 2.5, 3, 3.5, 4. 4.5, 5, 5.5, 6, 6.5, 7, 7.5, 8, 8.5, 9, 9.5, 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250 mg, and the like, or any range or value therein) per patient. A particular preferred daily dose is in the range of from about 0.3 to about 100 mg per patient.

Individual doses of compounds of the invention may be in the range of about 0.1 to about 100 mg (e.g. 0.3, 0.5, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100 mg, and the like, or any range or values therein).

When used herein in relation to a specific value (such as an amount), the term “about” (or similar terms, such as “approximately”) will be understood as indicating that such values may vary by up to 10% (particularly, up to 5%, such as up to 1%) of the value defined. It is contemplated that, at each instance, such terms may be replaced with the notation “±10%”, or the like (or by indicating a variance of a specific amount calculated based on the relevant value). It is also contemplated that, at each instance, such terms may be deleted.

In any event, the physician, or the skilled person, will be able to determine the actual dosage, which will be most suitable for an individual patient, which is likely to vary with the condition that is to be treated, as well as the age, weight, sex and response of the particular patient to be treated. The above-mentioned dosages are exemplary of the average case; there can, of course, be individual instances where higher or lower dosage ranges are merited, and such are within the scope of the present invention.

The benefits of using the compounds of the invention, for example via a combination of administrative routes, separately, and/or sequentially, and/or in parallel at the same time is to produce a tailored treatment for the patient in need of the therapy, with the possibility of preventing and/or reducing side effects, and also tune the correct dosage levels of a therapeutically effective amount of a compound of the invention.

The kits of parts described herein may comprise more than one composition or formulation including an appropriate quantity/dose of a compound of the invention, and/or more than one composition or formulation including an appropriate quantity/dose of the other therapeutic agent, in order to provide for repeat dosing. If more than one composition or formulation (comprising either active compound) is present, such compositions or formulations may be the same, or may be different in terms of the dose of either compound, chemical composition(s) and/or physical form(s).

Thus, in respect of the combination product according to the invention, the term “administration in conjunction with” includes that the two components of the combination product (compound of the invention and other therapeutic agent) are administered (optionally repeatedly), either together, or sufficiently closely in time, to enable a beneficial effect for the patient, that is greater, over the course of the treatment of the relevant condition, than if either a composition or formulation comprising compound of the invention, or a composition or formulation comprising the other agent, are administered (optionally repeatedly) alone, in the absence of the other component, over the same course of treatment. Determination of whether a combination provides a greater beneficial effect in respect of, and over the course of treatment of, a particular condition will depend upon the condition to be treated or prevented, but may be achieved routinely by the skilled person.

Further, in the context of a kit of parts according to the invention, the term “in conjunction with” includes that one or other of the two compositions or formulations may be administered (optionally repeatedly) prior to, after, and/or at the same time as, administration of the other component. When used in this context, the terms “administered simultaneously” and “administered at the same time as” include that individual doses of the relevant compound of the invention and other therapeutic agent are administered within 48 hours (e.g. 24 hours) of each other.

In further aspects of the invention, there is provided a process for the preparation of a combination product or kit-of-parts as hereinbefore defined, which process comprises bringing into association certain compounds of the invention, as hereinbefore defined, with the other therapeutic agent that is useful in the treatment of the relevant disease, disorder and/or condition, and at least one pharmaceutically-acceptable excipient.

Pharmaceutically-Acceptable Salts

The compounds of the present disclosure and/or of the invention may be provided in the form of a pharmaceutically acceptable salt. For instance, the pharmaceutically acceptable salt may be an acid addition salt or a base addition salt. The acid addition salt may be formed from a compound of Formula I and an acid, such as an organic acid. The organic acid may be trifluoroacetic acid, formic acid, acetic acid, benzoic acid, oxalic acid, fumaric acid, maleic acid, preferably, trifluoroacetic acid. The organic acid may also be a sulfonic acid, such as methanesulfonic acid, ethanesulfonic acid, toluenesulfonic acid, etc. The acid may also be an inorganic acid, such as hydrochloric acid, hydrobromic acid, etc. Examples of acid addition salts include salts comprising a cation of a compound of Formula I and the conjugate base of an acid such as CF₃C(O)O⁻. The base addition salt may be formed from a compound of Formula I and a base, such as an organic base. The base addition salts that may be mentioned include salts formed with alkali metals, such as Li, Na and K salts; alkaline earth metals, such as Mg and Ca salts; other metals, such as Al and Zn salts; or amine bases, such as ammonia, ethylenediamine, ethanolamine, diethanolamine, triethanolamine and tromethamine. Examples of base addition salts include salts comprising an anion of a compound of Formula I and a cation such as Na⁺ or K⁺.

Solvates

It is to be understood that the compounds of the present disclosure may exist in solvated forms such as solvates of the free compounds or solvates of a pharmaceutically acceptable salt of the compound. The term “solvate” is used herein to describe a molecular complex comprising a compound of the present disclosure, or a pharmaceutically acceptable salt thereof, and one or more pharmaceutically acceptable solvent molecules such as ethanol. The term “hydrate” is employed when the solvent is water. Thus, solvated forms may include monohydrate, dihydrate, hemihydrate, trihydrate, tetrahydrate and the like.

Prodrugs

The compounds of the present disclosure may be in the form of a prodrug. A prodrug is a compound which may have little or no pharmacological activity itself, but when such a compound is administered into or onto the body of a patient it is converted into a compound possessing pharmacological activity.

Polymorphs

Compounds of the present disclosure may exist in a continuum of solid states ranging from fully amorphous to fully crystalline. Compounds of the present disclosure may also exist as oils. Where compounds of Formula I exist in crystalline and part crystalline forms, such forms may include solvates, which are included in the scope of the invention. It is to be understood that all polymorphs, such as mixtures of polymorphs, are included within the scope of the claimed compounds.

Compounds of the invention may also exist in solution (i.e. in solution in a suitable solvent). For example, compounds of Formula I may exist in aqueous solution, in which case compounds of the invention may exist in the form of hydrates.

Compounds of the invention may contain double bonds and, unless otherwise indicated, may thus exist as E (entgegen) and Z (zusammen) geometric isomers about each individual double bond. Unless otherwise specified, all such isomers and mixtures thereof are included within the scope of the invention.

Compounds of the invention may also exhibit tautomerism. All tautomeric forms and mixtures thereof are included within the scope of the invention (particularly those of sufficient stability to allow for isolation thereof).

Compounds of the invention may also contain one or more asymmetric carbon atoms and may therefore exhibit optical and/or diastereoisomerism (i.e. existing in enantiomeric or diastereomeric forms). Diastereoisomers may be separated using conventional techniques, e.g. chromatography or fractional crystallisation. The various stereoisomers (i.e. enantiomers) may be isolated by separation of a racemic or other mixture of the compounds using conventional, e.g. fractional crystallisation or HPLC, techniques. Alternatively the desired enantiomer or diastereoisomer may be obtained from appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution; for example, with a homochiral acid followed by separation of the diastereomeric derivatives by conventional means such as chromatography), or by reaction with an appropriate chiral reagent or chiral catalyst, all of which methods and processes may be performed under conditions known to the skilled person. Unless otherwise specified, all stereoisomers and mixtures thereof are included within the scope of the invention.

Isotopically Labelled Compounds

Compounds of the present disclosure may be used in their labelled or unlabeled form. The compounds of the present disclosure may be isotopically labelled by having one or more of their atoms replaced with one or more of the following isotopes: 2H (deuterium), 3H (tritium), 11C, 13C, 14C, 18O, 17O, 19F, 18F.

Thus, compounds of the invention may have one of their atoms replaced by an atom having an atomic mass or mass number different from the atomic mass or mass number usually found in nature (or the most abundant one found in nature). All isotopes of any particular atom or element as specified herein are contemplated within the scope of the compounds of the invention.

Methods of Preparation

The compound of Formula I described herein may be prepared using methods known in the art and/or as described in this document.

It will be appreciated by those skilled in the art that, in the processes described above and hereinafter, the functional groups of intermediate compounds may need to be protected by protecting groups.

Functional groups that are desirable to protect include sulphonamido, amido, amino and aldehyde. Suitable protecting groups for sulphonamido, amido and amino include tert-butyloxycarbonyl, benzyloxycarbonyl, 2-trimethylsilylethoxycarbonyl (Teoc) or tert-butyl. Suitable protecting groups for aldehyde include alcohols, such as methanol or ethanol, and diols, such as 1,3-propanediol or, preferably, 1,2-ethanediol (so forming a cyclic acetal). The protection and deprotection of functional groups may take place before or after a reaction as outlined in Scheme 1 below.

Protecting groups may be applied and removed in accordance with techniques that are well-known to those skilled in the art and as described hereinafter. For example, protected compounds/intermediates described herein may be converted chemically to unprotected compounds using standard deprotection techniques. The type of chemistry involved will dictate the need, and type, of protecting groups as well as the sequence for accomplishing the synthesis. The use of protecting groups is fully described in “Protective Groups in Organic Synthesis”, 3rd edition, T. W. Greene & P. G. M. Wutz, Wiley-Interscience (1999), the contents of which are incorporated herein by reference.

For instance, a compound of Formula I may be synthesized as outlined in Scheme 1 below.

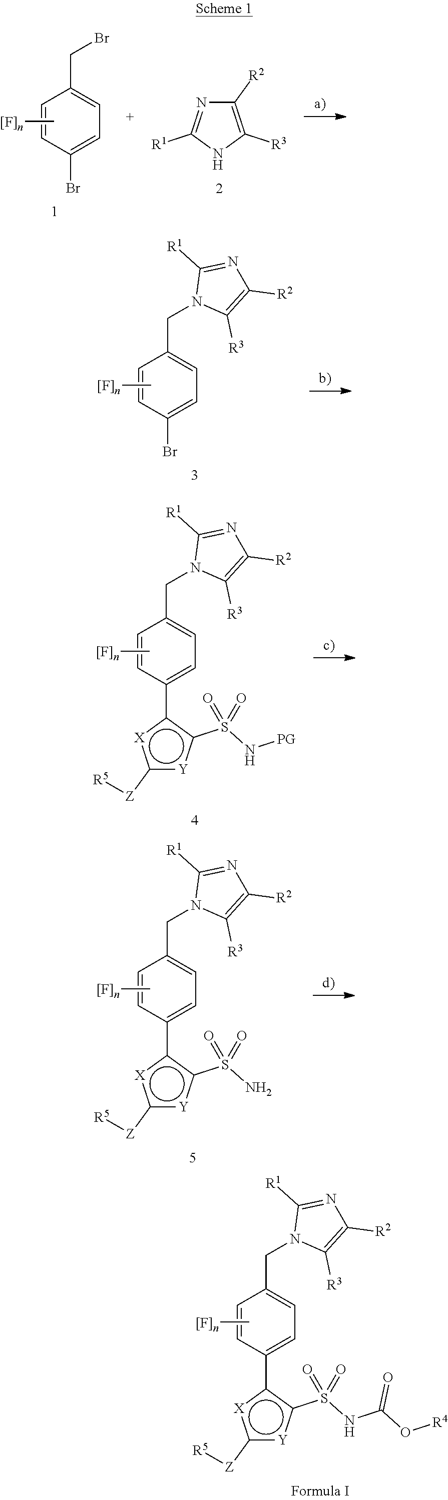

In Scheme 1, the dibromide 1 is reacted with the heterocyclic compound 2 under reaction conditions a) to provide the compound 3. The dibromide 1 may be substituted with 0, 1, 2, 3 or 4 F, i.e. n may be 0, 1, 2, 3 or 4. The heterocyclic compound 2 is substituted with the substituents R¹, R² and R³ which may have the values indicated for the compound of Formula I described herein. Thus, the heterocyclic compound 2 may be a substituted imidazole. The compound 3 is subsequently subjected to reaction conditions b) to provide the protected sulfone amide 4. The values X, Y and R⁵ for the protected sulfone amide 4 may be as for the compound of Formula I described herein, and PG is a protecting group such as tert-butyl. The moiety containing X and Y of the compound of Formula I may be a phenyl ring, a thiophene ring or a thiazole ring. The reaction conditions b) may involve Suzuki cross coupling conditions. This is followed by removal of the protecting group and conversion of the resulting sulfone amide 5 into the compound of Formula I using the reaction conditions d).

The reaction conditions used in a), b) and c) may vary depending on the final chemical structure to be synthesized.

For instance, a compound of Formula I containing an imidazole and in which the X and Y containing moiety is a phenyl ring, R⁵-Z represents isobutyl and R⁴ represents C₁-C₆ alkyl substituted with 0, 1, 2 or 3 F, may be prepared using the following reaction conditions:

a) NaH, N,N′-dimethylformamide, 0° C. to room temperature, reaction performed overnight,

b) 2-(N-tert-butyl)sulfamoyl)-5-isobutylphenyl)boronic acid, Pd(PPh₃)₄, K₂CO₃, toluene, ethanol, water, 120° C. microwave irradiation, 1 h,

c) trifluoroacetic acid, room temperature, overnight; and

d) triethylamine, the appropriate alkyl chloroformate, dichloromethane, room temperature, 2 h.

It will be appreciated that the boronic acid of step b) may be prepared as reported in

Bioorg. Med. Chem. Lett. 2018, 28 (3), 519-522. Alternatively, the synthesis may involve a boronic acid ester such as a MIDA derivate or a pinacol ester.

It is understood that the values for R¹, R², R³, R⁴, R⁵, X, Y and Z may be as described for the compounds disclosed in this document such as a compound of Formula I.

The present disclosure also provides a compound of Formulae 4 or 5 as described herein. The compound of Formulae 4 or 5 may serve as an intermediate in the preparation of a compound of Formula I as described herein.

### EXAMPLES

The following abbreviations are used throughout this document.

Abbreviations

ACN Acetonitrile

DAST Diethylaminosulfur trifluoride

DCM Dichloromethane

DMA N,N′-Dimethylacetamide

DMF N,N′-Dimethylformamide

Dppf 1,1′-Ferrocenediyl-bis(diphenylphosphine)

EA Ethyl Acetate

FCC Flash Column Chromatography

h hour(s)

HFBA HeptaFluoroButyric Acid

HLM Human Liver Microsome

HPLC High Performance Liquid Chromatography

HRMS (ESI) High Resolution Mass Spectrosopy (Electrospray Ionization)

Hz Hertz

μM micro molar

MHz Mega Hertz

LCM Liquid Chromatography

LCMS Liquid Chromatography Mass Spectroscopy

MIDA Methyl-IminoDiAcetic Acid

min minute(s)

MLM Mouse Liver Microsome

NMR Nuclear Magnetic Resonance

PA Propionic Acid

PBS Phosphate-Buffered Saline

RLM Rat Liver Microsome

TFA TrifluoroAcetic Acid

Xantphos 4,5-Bis(diphenylphosphino)-9,9-dimethylxanthene

The naming of the compounds in this document has been made using ChemDraw Professional 15.0 or ChemDraw Professional 16.0. In this document, if the chemical name and the chemical structure are inconsistent the chemical structure should be considered to be correct.

General

All chemicals and solvents were purchased from Sigma Aldrich, Fisher Scientific, FluoroChem, and Enamine, and were used without further purification. Microwave heating was performed in a Biotage single-mode microwave reactor producing controlled irradiation at 2450 MHz with a power of 0-400 W. The reaction temperature was determined and controlled using the built-in online IR-sensor. Microwave mediated reactions were performed in septum sealed vials designed for 0.5-2.0 mL, 2.0-5.0 mL, or 10-20 mL reaction volumes. Commercial Isolute HM-N (diatomaceous earth) prepacked columns was used to remove traces of water. Automated flash column chromatography was performed on Biotage Isolera or Grace Reveleris instruments using commercial silica cartridges. Manual flash chromatography was performed on silica gel 60 (40-63 μm). Preparative reverse-phase HPLC was performed using a C18 column (Macherey-Nagel VP 125/21 Nucleodur C18 HTec 5 μm) with UV (254 nm) detection and a mobile phase of acetonitrile in 0.05% aqueous formic acid, or acetonitrile in 0.1% aqueous trifluoroacetic acid.

Analytical HPLC/ESI-MS was performed using electrospray ionization (ESI) and a C18 column (50×3.0 mm, 2.6 μm particle size, 100 Å pore size) with gradients of acetonitrile in 0.05% aqueous formic acid as mobile phase at a flow rate of 1.5 ml/min. High resolution molecular masses (HRMS) were determined on a mass spectrometer equipped with an ESI source and 7-T hybrid linear ion trap (LTQ). Nuclear magnetic resonance (NMR) spectra were recorded on Varian instruments at 400 MHz and 500 MHz for ¹H, 101 MHz and 126 MHz for ¹³C and 376 MHz for ¹⁹F. Chemical shifts (δ) are reported in ppm with the residual solvent peak as internal standard (¹H: chloroform-d at 7.26 ppm, DMSO-d6 at 2.50 ppm, acetone-d6 at 2.05 ppm, methanol-d4 at 3.31 ppm; ¹³C: chloroform-d at 77.2 ppm, DMSO-d6 at 39.5 ppm; ¹⁹F: FCCl₃ capillary at 0.00 ppm). Coupling constants (J) are reported in hertz (Hz). All final compounds, unless specifically noted, were ≥95% pure as determined by analytical HPLC and/or NMR.

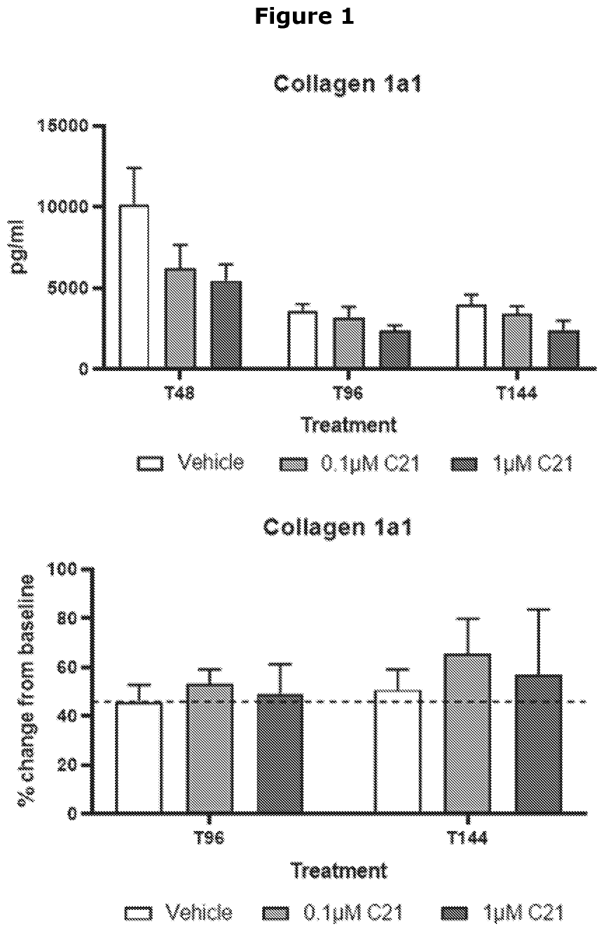

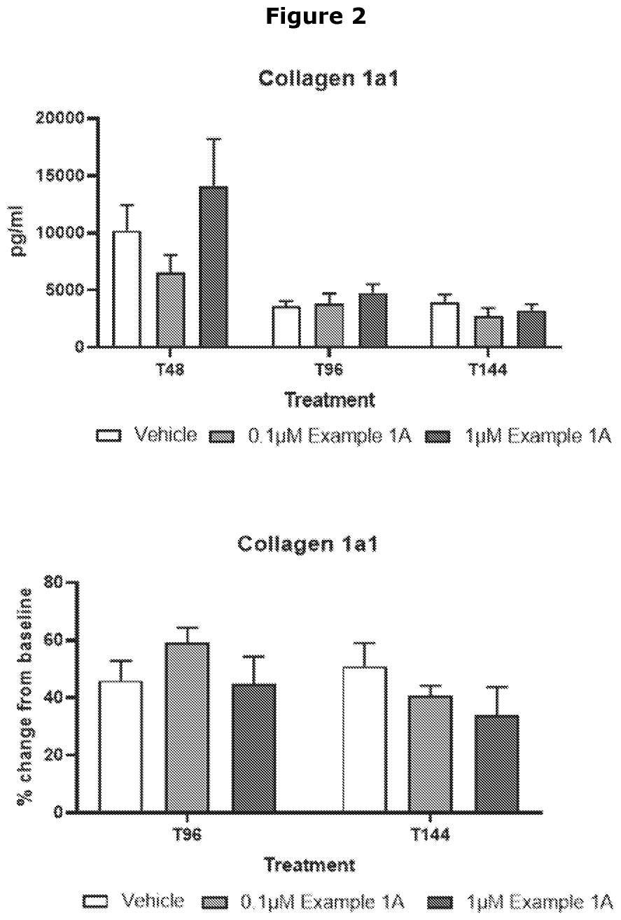

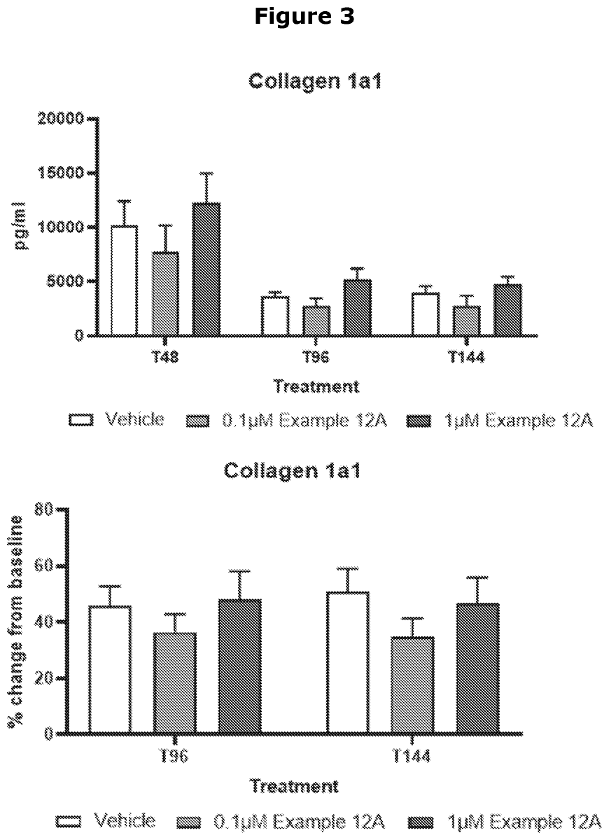

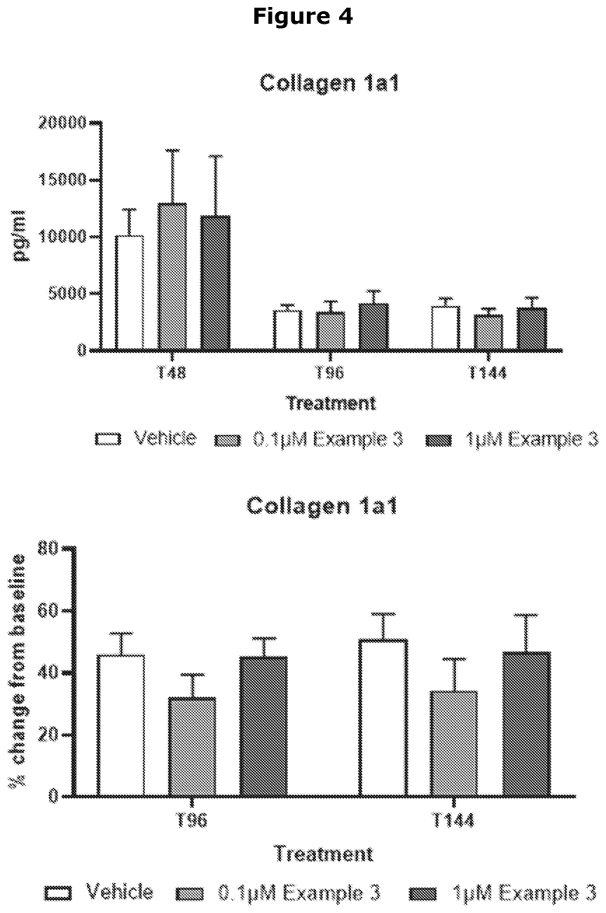

The invention is illustrated, but in no way limited, by the following examples with reference to the attached figures, in which: FIGS. 1 to 4 show secreted Col1a1

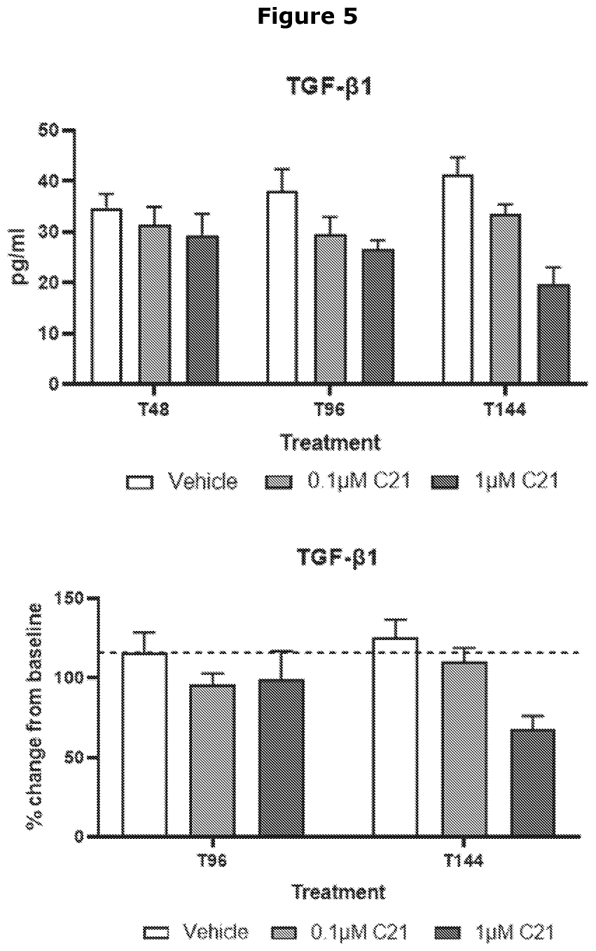

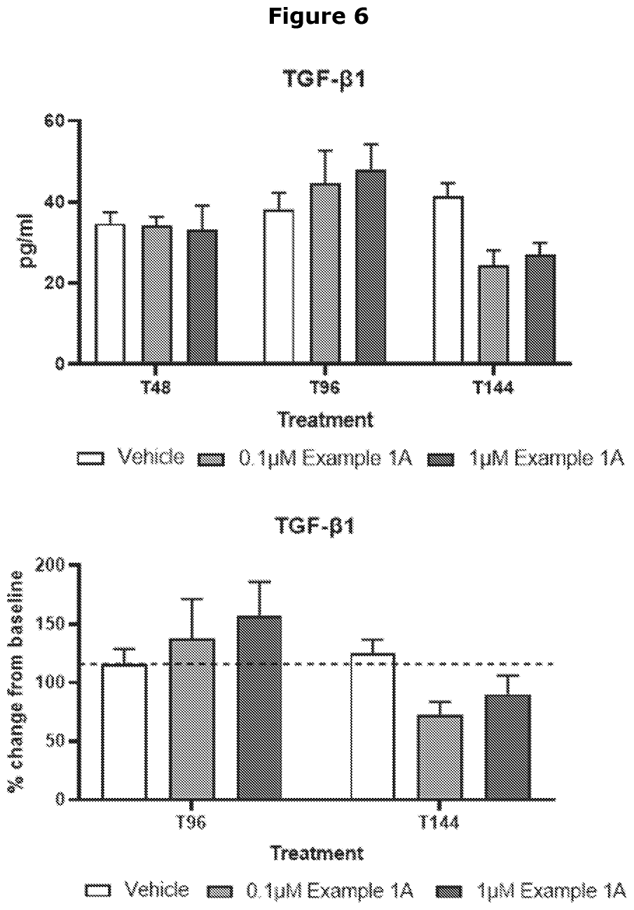

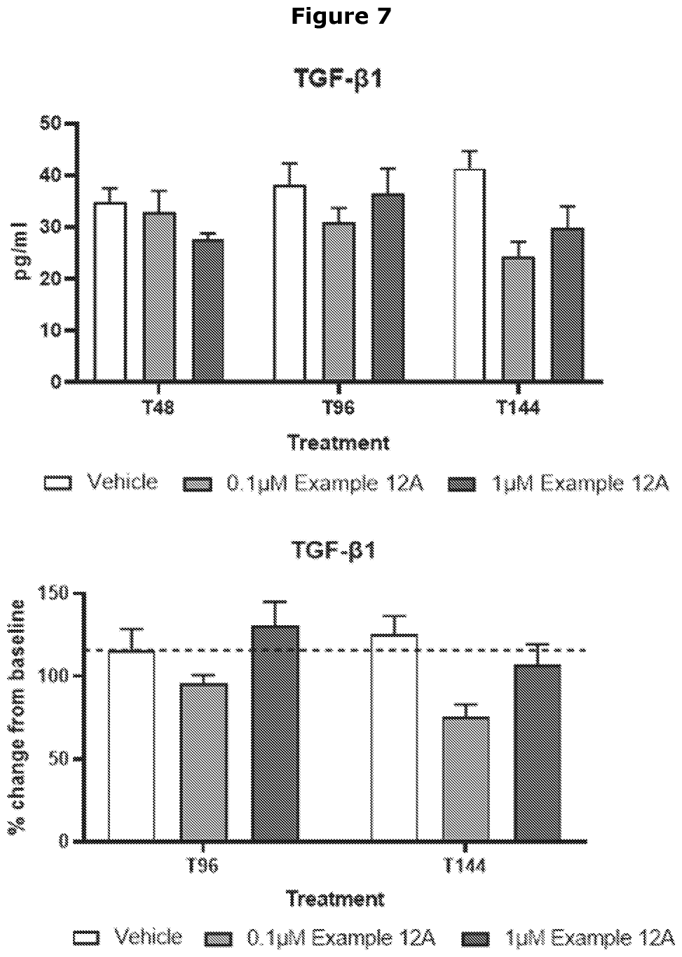

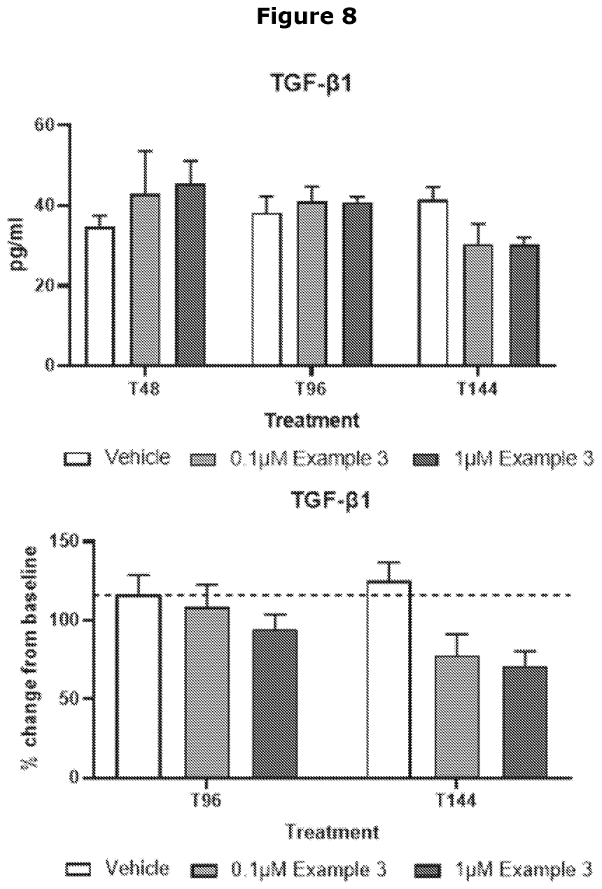

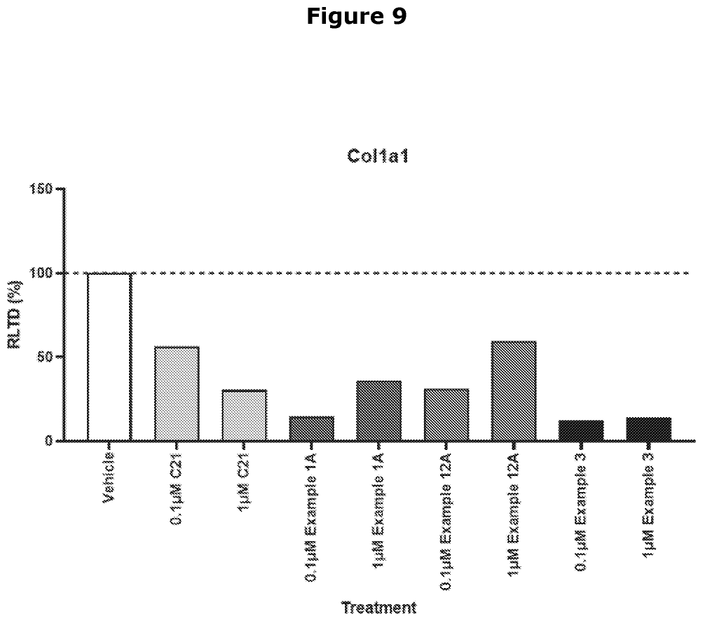

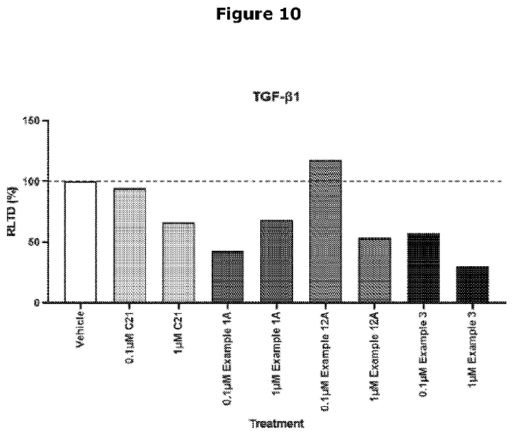

(Col1a1) levels in PCLus culture supernatants for C21, Example 1A, Example 12A and Example 3 at 48, 96 and 144 hours. FIGS. 5 to 8 show secreted TGF-β1 levels in PCLus culture supernatants for C21, Example 1A, Example 12A and Example 3 at 48, 96 and 144 hours. FIGS. 9 and 10 show selected gene transcript levels in PCLus for Col1a1and TGF-β1 (normalised to (β-Actin) with C21, Example 1A, Example 12A and Example 3 treatment at 144 h.

Preparation of Starting Materials for Examples 1-2

2-tert-butyl-1H-imidazole was purchased from Fluorochem.

N-alkylation of heterocycles

1-[(4-Bromophenyl)methyl]-2-tert-butyl-imidazole

NaH (0.184 g, 8.0 mmol, 2 equiv.) was added to a stirred solution of 2-tert-butyl-1H-imidazole (0.497 g, 4.0 mmol, 1 equiv.) in DMF (0.27 M) at 0° C. After 20 min 1-bromo-4-(bromomethyl)benzene (1.05 g, 4.2 mmol, 1.05 equiv.) was added. The resulting mixture was allowed to warm to ambient temperature and stirred overnight, then quenched with water (15 mL). The product was extracted with ethyl acetate (3×25 mL). The combined organic layers were washed with brine (20 mL), dried over anhydrous MgSO₄ and concentrated in vacuo. The crude product was obtained in 99% yield (1.16 g, 0.396 mmol) without need of trituration.

Suzuki cross coupling of MIDA boronate and the N-alkylated heterocycles

N-tert-Butyl-2-[4-[(2-tert-butylimidazol-1-yl)methyl]phenyl]-4-isobutyl-benzenesulfonamide

N-(tert-butyl)-4-isobutyl-2-(6-methyl-4,8-dioxo-1,3,6,2-dioxazaborocan-2-yl)benzenesulfonamide (0.441 g, 1.04 mmol, 1.04 mmol), potassium carbonate (0.083 g, 0.6 mmol, 1.5 equiv.) and tetrakis(triphenylphosphine)palladium(0) (0.231 g, 0.02 mmol, 0.05 equiv.) was suspended in toluene (0.18 M). To this mixture was added 1-[(4-bromophenyl)methyl]-2-tert-butyl-imidazole (0.293 g, 1.0 mmol, 1 equiv.) in ethanol (0.5 M). Water (0.25 mL). The resulting reaction mixture was stirred at 120° C. for 60 min under microwave irradiation, the allowed to cool to ambient temperature, water (10 mL) was added and the reaction mixture extracted with ethyl acetate (2×25 mL). The combined organic layers were washed with brine (25 mL), dried over anhydrous MgSO₄ and concentrated. The crude product was purified by FCC (15-30% EA in iso-hexane) to afford the title compound as a solid in 36% yield (0.171 g, 0.355 mmol).

Deprotection of the sulfonamides

2-[4-[(2-tert-butylimidazol-1-yl)methyl]phenyl]-4-isobutyl-benzenesulfonamide

N-tert-butyl-2-[4-[(2-tert-butylimidazol-1-yl)methyl]phenyl]-4-isobutyl-benzenesulfonamide (0.135 g, 0.28 mmol) was stirred in trifluoroacetic acid (4.6 mL) at ambient temperature overnight. The reaction was diluted with water (10 mL) and the product extracted with ethyl acetate (2×20 mL). The combined organic layers were washed with brine (5 mL), dried over anhydrous MgSO₄ and concentrated in vacuo. The crude sulfonamide was purified by FCC (5% MeOH in DCM) to afford the product as a white amorphous solid in 86% yield (0.102 g, 0.24 mmol).

Preparation of Starting Materials for Examples 3-6

General procedure for the synthesis of thiazole sulfonamides
a) 2-Alkylation of 2,4-dibromothiazole

Under nitrogen atmosphere Pd(OAc)₂ (0.03 mmol, 0.03 equiv.), was combined with xantphos (0.05 mmol, 0.05 equiv.) in dry THF. After stirring for 5 min, this solution was transferred to a separate vessel under nitrogen containing 2,4-dibromothiazole (1.03 mmol, 1 equiv.) and alkylzinc bromide (0.5 M in THF, 1.08 mmol, 1.05 equiv.).

The sealed vessel was heated at 80° C. for 16 h. Upon cooling to room temperature, the insoluble solid was removed by filtration through a Celite pad (eluting with CH₂Cl₂). The filtrate was diluted with H₂O (50 mL) and extracted with CH₂C₁₂ (3×50 mL). The combined organic extracts were washed with brine (30 mL), dried over MgSO₄ and concentrated in vacuo to give the crude product. The crude product was purified by FCC (0-10% ethyl acetate in iso-hexane) to afford the corresponding products in 55-65% yield.
b) Synthesis of sulfonic acid derivative of thiazoles

4-Bromo-2-alkylated thiazole (1 mmol, 1 equiv.) was dissolved in DMF (2 mL), after cooling to 5° C., chlorosulfonic acid was added slowly in such degree (5 mmol, 5 equiv.). Afterwards, the mixture was stirred at 120° C. for 16 h. Upon cooling to room temperature, the solvent was removed under reduced pressure and the crude material was purified by column chromatography (10% MeOH in DCM) to afford the corresponding product.
c) Synthesis of sulfonyl chloride from sulfonic acids

4-Bromo-2-alkylated thiazole-5-sulfonic acid (1 mmol, 1 equiv.) was dissolved in 10 mL of DCM and PCIS (2 mmol, 2 equiv.) was added slowly and the reaction mixture was stirred for overnight at 60° C. The reaction was quenched with water (50 mL), extracted with ethyl acetate (2×50 mL). The combined organic layers were washed with water (30 mL), brine (25 mL) and dried over anhydrous MgSO₄ and concentrated in vacuo. The crude product was purified by FCC (0-25% Ethyl acetate in iso-hexane) to afford the chloride intermediate in 80-95% yield.
d) Synthesis of 4-bromo-N-(tert-butyl)-2-alkylated thiazole-sulfonamide

The chloride intermediate (1 mmol, 1 equiv.) was dissolved in DCM (2 mL) and tertbutylamine (1.1 mmol, 1.1 equiv.) was added and the reaction mixture was stirred for overnight under room temperature. The reaction mixture was then extracted with DCM (2×50 mL). The combined organic layers were washed with water (30 mL), brine (25 mL) and dried over anhydrous MgSO₄ and concentrated in vacuo. The crude product was purified by FCC (0-30% ethyl acetate in iso-hexane) to afford the corresponding sulfonamide in 90-95% yield.

N-alkylation of 2-tert-butyl imidazole

The 2-tert-butyl imidazole (6.0 mol, 1 equiv.), bromobenzyl bromide (6 mol, 1 equiv.) and base NaH (6 mol, 1 equiv.) in acetonitrile (10 mL) was stirred at room temperature for overnight. The reaction was quenched with water (50 mL), extracted with ethyl acetate (2×50 mL). The combined organic layers were washed with water (30 mL), brine (25 mL) and dried over anhydrous MgSO₄ and concentrated in vacuo. The crude product was purified by FCC (50-100% ethyl acetate in iso-hexane) to afford the

N-alkylated heterocycles in 80-95% yield.

Synthesis of pinacolato-boronate esters

Pd(dppf)Cl₂ (0.03 mmol, 0.03 equiv.) was added to a mixture of 2-tert-butyl imidazole (1.0 mmol, 1 equiv.), KOAc (3.0 mmol, 3 equiv.) and B₂pin₂ (3.5 mmol, 3.5 equiv.) in DMF (5 mL). The mixture was stirred at 80° C. under N2 for 16 hours. The insoluble solid was removed by filtration through a Celite pad (eluting with CH₂Cl₂). The filtrate was diluted with H₂O (50 mL) and extracted with CH₂Cl₂ (3×50 mL). The combined organic extracts were washed with brine (30 mL), dried over MgSO₄ and concentrated in vacuo to give the crude product. The crude product was purified by FCC (50-100% ethyl acetate in iso-hexane) to afford the corresponding product in 80-95% yield.

General procedure for the Suzuki cross coupling of thiazole sulfonamide and boronate ester of the N-alkylated heterocycles

Thiazole sulfonamide (1 mmol, 1 equiv.), N-alkylated product (1 mmol, 1 equiv.), potassium carbonate (4 mmol, 4 equiv.) and Pd(dppf)C12 (0.05 mmol, 0.05 equiv.) was suspended in 1,2-dimethoxyethane (3 mL) and water (0.6 mL). The vial which was sealed and the resulting reaction mixture was stirred at 120° C. for 60 min under microwave irradiation. The mixture was allowed to cool to ambient temperature, then extracted with chloroform (3×2 mL). The combined organic layers were washed with brine (25 mL), dried over anhydrous MgSO₄ and concentrated. The crude product was isolated through manual FCC (0-10% MeOH in DCM) to afford the products in 70-85% yield.

General procedure for deprotection of the sulfonamide product

The Suzuki coupled product (0.15 mmol) was stirred in trifluoroacetic acid (2.5 mL) at 50° C. for overnight. The reaction was diluted with water (10 mL) and the product was extracted with dichloromethane (2×20 mL). The combined organic layers were washed with brine (15 mL), dried over anhydrous MgSO₄ and concentrated. The sulfonamide was used purified through a small silica plug (0-5% with MeOH in acetonitrile) and used in the following carbamate formation reactions without further purification.

### EXAMPLE 1A

### Methyl ((4′-((2-(tert-butyl)-1H-imidazol-1-yl)methyl)-5-isobutyl-[1,1′-biphenyl]-2-yl)sulfonyl)carbamate

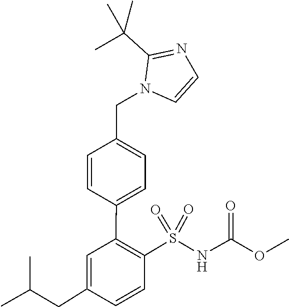 [SMILES: COC(=O)NS(=O)(=O)c1ccc(CC(C)C)cc1-c1ccc(Cn2ccnc2C(C)(C)C)cc1]

Methyl ((4′-((2-(tert-butyl)-1H-imidazol-1-yl)methyl)-5-isobutyl-[1,1′-biphenyl]-2-yl)sulfonyl)carbamate was synthesized as described. The crude sulfonamide (55.3 mg, 0.13 mmol, 1 equiv.) was dissolved in DCM (0.05 M). Triethyl amine (90 μL, 0.65 mmol, 5 equiv.) and methyl chloroformate (11 μL, 0.14 mmol, 1.1 equiv.) was added. The reaction was stirred at room temperature for 2 h. The reaction mixture was diluted with ethyl acetate (10 mL), extracted with ethyl acetate (3×5 mL), washed with brine (3 mL), dried over MgSO₄ and concentrated. The crude product was purified by HPLC (30-70° A) ACN in water with 0.05% formic acid). After lyophilisation the product was obtained as a white amorphous solid (17 mg, 27% yield). ¹H NMR (500 MHz, Acetone-d₆) δ 8.11 (d, J=8.2 Hz, 1H), 7.43 (dd, J=8.3, 1.8 Hz, 1H), 7.41-7.36 (m, 2H), 7.18-7.09 (m, 3H), 6.91 (d, J=1.3 Hz, 1H), 6.83 (d, J=1.3 Hz, 1H), 5.48 (s, 2H), 3.54 (s, 2H), 2.62 (d, J=7.2 Hz, 2H), 2.02-1.91 (m, 1H), 1.41 (s, 9H), 0.94 (d, J=6.6 Hz, 6H). ¹³C NMR (126 MHz, Acetone-d₆) δ 154.6, 152.7, 148.1, 141.5, 139.7, 138.6, 136.7, 134.0, 131.1, 130.3, 129.1, 126.8, 126.7, 122.8, 53.1, 51.2, 45.3, 34.1, 30.8, 30.4, 22.6. HRMS (ESI) calcd. for C₂₆H₃₄N₃O₄S⁺ [M+H]⁺ 484.2270 found 484.2269.

### EXAMPLE 1B

### Potassium ((4′-((2-(tert-butyl)-1H-imidazol-1-yl)methyl)-5-isobutyl-[1,1′-biphenyl]-2-yl)sulfonyl) (methoxycarbonyl)amide

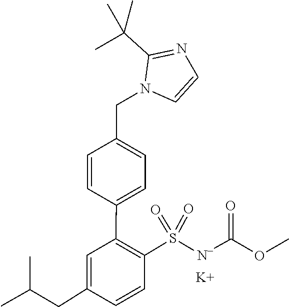 [SMILES: COC(=O)[N-]S(=O)(=O)c1ccc(CC(C)C)cc1-c1ccc(Cn2ccnc2C(C)(C)C)cc1 ; [K+]]

Potassium ((4′-((2-(tert-butyl)-1H-imidazol-1-yl)methyl)-5-isobutyl-[1,1′-biphenyl]-2-yl)sulfonyl)(methoxycarbonyl)amide was prepared as described. To a stirred solution of methyl ((4′-((2-(tert-butyl)-1H-imidazol-1-yl)methyl)-5-isobutyl-[1,1′-biphenyl]-2-yl)sulfonyl)carbamate (45.9 mg, 95 μmol, 1 equiv.) in chloroform (95 μM) was added KOH (11.1 mg, 0.198 mmol, 2 equiv.) in MeOH (0.59 M). The resulting solution was stirred at rt for 4 h, during which period precipitation of white solids was observed. The crude product was partially evaporated and triturated from chloroform with pentane (50 The resulting was solids were filtered, then dissolved in water (5 mL), The solution was filtered to ensure removal of organic residues. The product was obtained after lyophilisation as a white amorphous solid in 93% yield (46.1 mg, 88 μmol). ¹H NMR (400 MHz, DMSO-d₆) δ 7.86 (d, J=8.0 Hz, 1H), 7.53-7.28 (m, 2H), 7.13 (dd, J=8.1, 1.9 Hz, 1H), 7.02-6.90 (m, 3H), 6.86 (d, J=1.8 Hz, 1H), 6.76 (d, J=1.2 Hz, 1H), 5.38 (s, 2H), 3.15 (s, 3H), 2.46 (d, J=7.1 Hz, 2H), 1.84 (dt, J=13.6, 6.8 Hz, 1H), 1.34 (s, 9H), 0.87 (d, J=6.6 Hz, 6H). LCMS calc. for C₂₆H₃₄N₃O₄S⁺ [M+H]⁺ 484.2 found 484.1.

### EXAMPLE 2A

### Butyl ((4′-((2-(tert-butyl)-1H-imidazol-1-yl)methyl)-5-isobutyl-[1,1′-biphenyl]-2-yl)sulfonyl)carbamate

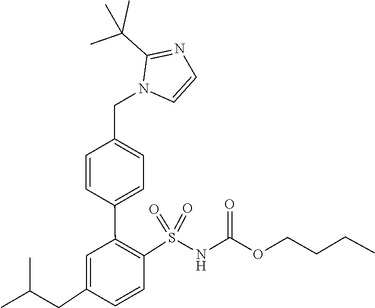 [SMILES: CCCCOC(=O)NS(=O)(=O)c1ccc(CC(C)C)cc1-c1ccc(Cn2ccnc2C(C)(C)C)cc1]

Butyl ((4′-((2-(tert-butyl)-1H-imidazol-1-yl)methyl)-5-isobutyl-[1,1′-biphenyl]-2-yl)sulfonyl)carbamate was synthesized as described for methyl ((4′-((2-(tert-butyl)-1H-imidazol-1-yl)methyl)-5-isobutyl-[1,1′-biphenyl]-2-yl)sulfonyl)carbamate using the crude sulfonamide (21.3 mg, 50 μmol) but butyl chloroformate (8.8 μL, 69.2 μmol). The crude product was purified by FCC (5% MeOH in DCM) followed by mini prep TLC (10% MeOH in DCM) to afford the product as a white amorphous solid (5 mg, 21% yield) after trituration with iso-hexane from DCM. ¹H NMR (400 MHz, Acetone-d₆) δ 8.11 (d, J=8.2 Hz, 1H), 7.44 (dd, J=8.2, 1.9 Hz, 1H), 7.37 (d, J=8.2 Hz, 2H), 7.24-7.00 (m, 3H), 6.88 (d, J=1.3 Hz, 1H), 6.80 (d, J=1.3 Hz, 1H), 5.47 (s, 2H), 3.96 (t, J=6.5 Hz, 2H), 2.62 (d, J=7.2 Hz, 2H), 2.03-1.86 (m, 1H), 1.51-1.39 (m, 2H), 1.28-1.16 (m, 2H), 0.93 (d, J=6.6 Hz, 6H), 0.85 (t, J=7.4 Hz, 3H). ¹³C NMR (101 MHz, Acetone-d₆) δ 154.6, 151.6, 148.3, 141.6, 139.5, 138.8, 136.5, 134.1, 131.1, 130.3, 129.2, 126.9, 126.9, 122.8, 66.5, 51.2, 45.3, 34.1, 31.3, 30.8, 30.4, 22.5, 19.5, 13.9. HRMS (ESI) calcd. for C₂₉H₄₀N₃O₄S⁺ [M+H]⁺ 526.2740 found 526.2731.

### EXAMPLE 2B

### Potassium ((4′-((2-(tert-butyl)-1H-imidazol-1-yl)methyl)-5-isobutyl-[1,1′-biphenyl]-2-yl)sulfonyl)(butoxycarbonyl)amide

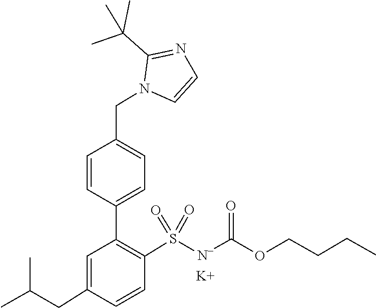 [SMILES: CCCCOC(=O)[N-]S(=O)(=O)c1ccc(CC(C)C)cc1-c1ccc(Cn2ccnc2C(C)(C)C)cc1 ; [K+]]

The title compound was synthesized as described for Example 1B using butyl ((4′-((2-(tert-butyl)-1H-imidazol-1-yl)methyl)-5-isobutyl-[1,1′-biphenyl]-2-yl)sulfonyl)-carbamate (20.4 mg, 38.5 μmol, 1.0 equiv.) and KOH (2.33 mg, 41.6 μmol, 1.08 equiv.) in MeOH (0.59 M). The product was obtained as a white amorphous solid (16.5 mg, 76% yield). ¹1H NMR (400 MHz, DMSO-d₆) 57.85 (d, J=8.1 Hz, 1H), 7.45-7.35 (m, 2H), 7.12 (dd, J=8.1, 1.8 Hz, 1H), 6.94 (dd, J=4.7, 3.4 Hz, 3H), 6.84 (d, J=1.8 Hz, 1H), 6.76 (d, J=1.2 Hz, 1H), 5.37 (s, 2H), 3.54 (t, J=6.5 Hz, 2H), 2.45 (d, J=7.1 Hz, 2H), 1.91-1.74 (m, 1H), 1.34 (s, 9H), 1.30 (d, J=6.8 Hz, 2H), 1.27-1.13 (m, 2H), 0.87 (d, J=6.6 Hz, 6H), 0.83 (t, J=7.3 Hz, 3H). LCMS calc. for C₂₉H₄₀N₃O₄S⁺ [M+H]⁺ 526.2734 found 526.2.

### EXAMPLE 3

### Methyl ((4-(4-((2-(tert-butyl)-1H-imidazol-1-yl)methyl)phenyl)-2-isobutylthiazol-5-yl)sulfonyl) carbamate

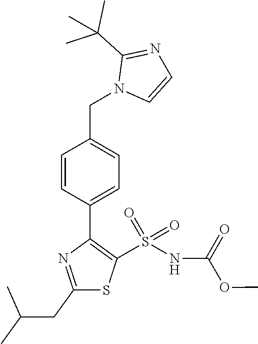 [SMILES: COC(=O)NS(=O)(=O)c1sc(CC(C)C)nc1-c1ccc(Cn2ccnc2C(C)(C)C)cc1]

To a mixture of the corresponding sulfonamide (0.12 mmol, 1 equiv.) and triethylamine (0.58 mmol, 5 equiv.) in DCM (2 mL), methyl chloroformate (0.17 mmol, 1.5 equiv.) was added drop-wise over approximately 2 min and the reaction mixture was stirred at room temperature for 20 min. The reaction mixture was quenched with water and diluted with DCM (25 mL), then extracted with 10% citric acid (aq., 20 mL), water (3×30 mL) and brine (20 mL) in sequence. The combined organic layer was dried over MgSO₄ and concentrated. The crude product was purified by manual FCC (0-5% with MeOH in acetonitrile) and the product was obtained as white amorphous solid (40 mg, 71% yield). ¹1H NMR (400 MHz, Acetone-d₆) δ 8.26 (d, J=8.4 Hz, 2H), 7.13 (d, J=8.4 Hz, 2H), 7.09 (d, J=1.5 Hz, 1H), 7.03 (d, J=1.5 Hz, 1H), 5.56 (s, 2H), 3.40 (s, 3H), 2.83 (d, J=7.1 Hz, 2H), 2.19-2.09 (m, 1H), 1.45 (s, 9H), 1.02 (d, J=6.7 Hz, 6H). ¹³C NMR (101 MHz, Acetone-d₆) δ 168.5, 160.3, 153.5, 150.7, 137.7, 137.4, 133.9, 130.4, 125.6, 124.0, 122.7, 51.5, 50.8, 41.6, 33.4, 29.3, 29.1, 21.7. HRMS (ESI): calcd. for C₂₃H₃₁N₄O₄S₂⁺ [M+H]⁺ 491.1781; found: 491.1795.

### EXAMPLE 4

### Butyl ((4-(4-((2-(tert-butyl)-1H-imidazol-1-yl)methyl)phenyl)-2-isobutylthiazol-5-yl)sulfonyl) carbamate

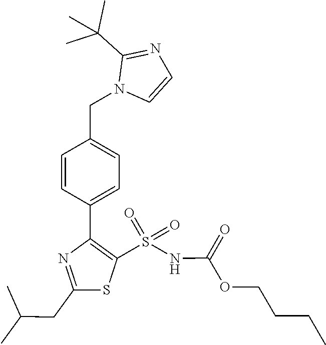 [SMILES: CCCCOC(=O)NS(=O)(=O)c1sc(CC(C)C)nc1-c1ccc(Cn2ccnc2C(C)(C)C)cc1]

To a mixture of the corresponding sulfonamide (0.12 mmol, 1 equiv.) and triethylamine (0.58 mmol, 5 equiv.) in DCM (2 mL), butyl chloroformate (0.17 mmol, 1.5 equiv.) was added drop-wise over approximately 2 min and the reaction mixture was stirred at room temperature for 20 min. The reaction mixture was quenched with water and diluted with DCM (25 mL), then extracted with 10° A) citric acid (aq., 20 mL), water (3×30 mL) and brine (20 mL) in sequence. The combined organic layer was dried over MgSO₄ and concentrated. The crude product was purified by manual FCC (0-5% with MeOH in acetonitrile) and the product was obtained as white amorphous solid (39 mg, 63% yield). ¹H NMR (400 MHz, Chloroform-d) δ 7.98 (d, J=8.1 Hz, 2H), 7.03 (d, J=8.1 Hz, 2H), 6.92 (d, J=1.5 Hz, 1H), 6.80 (d, J=1.5 Hz, 1H), 5.30 (s, 2H), 3.81 (t, J=6.8 Hz, 2H), 2.77 (d, J=7.1 Hz, 2H), 2.15-2.04 (m, 1H), 1.44-1.40 (m, 11H), 1.23-1.14 (m, 2H), 0.97 (d, J=6.6 Hz, 6H), 0.80 (t, J=7.3 Hz, 3H). ¹³C NMR (101 MHz, Chloroform-d) δ 170.5, 160.3, 153.9, 152.5, 137.0, 135.1, 133.7, 130.3, 126.1, 124.4, 122.5, 66.0, 51.0, 42.2, 33.5, 30.9, 29.6, 29.6, 22.3, 19.0, 13.8. HRMS (ESI): calcd. for C₂₆H₃₇N₄O₄S₂⁺ [M+H]⁺533.2251; found: 533.2241.

### EXAMPLE 5

### Methyl ((4-(4-((2-(tert-butyl)-1H-imidazol-1-yl)methyl)phenyl)-2-propylthiazol-5-yl)sulfonyl)carbamate

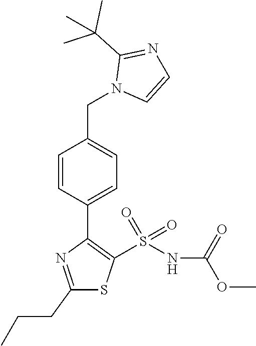 [SMILES: CCCc1nc(-c2ccc(Cn3ccnc3C(C)(C)C)cc2)c(S(=O)(=O)NC(=O)OC)s1]

To a mixture of sulfonamide (0.12 mmol, 1 equiv.) and triethylamine (0.60 mmol, 5 equiv.) in DCM (2 mL), methyl chloroformate (0.14 mmol, 1.2 equiv.) was added drop-wise over approximately 2 min and the reaction mixture was stirred at room temperature for 20 min. The reaction mixture was quenched with water and diluted with DCM (25 mL), then extracted with 10° A) citric acid (aq., 20 mL), water (3×30 mL) and brine (20 mL) in sequence. The combined organic layer was dried over MgSO₄ and concentrated. The crude product was purified by manual FCC (0-5% with MeOH in acetonitrile) and the product was obtained as white amorphous solid (28 mg, 49% yield). ¹H NMR (400 MHz, Acetone-d₆) δ 8.12 (d, J=8.4 Hz, 2H), 7.03 (d, J=3.7 Hz, 2H), 7.01-6.96 (m, 2H), 5.47 (s, 2H), 5.23 (s, 1H), 3.27 (s, 3H), 2.79 (t, J=7.2 Hz, 2H), 1.77-1.60 (m, 2H), 1.34 (s, 9H), 0.89 (t, J=7.4 Hz, 3H). ¹³C NMR (101 MHz, Acetone-d₆) δ 169.7, 160.4, 153.4, 150.7, 137.5, 137.0, 134.0, 130.4, 125.7, 123.1, 123.0, 51.6, 51.0, 34.7, 33.5, 28.9, 22.8, 13.1. HRMS (ESI): calcd. for C₂₂H₂₉₄O₄S₂⁺ [M+H]⁺ 477.1625; found: 477.1631.

### EXAMPLE 6

### Butyl ((4-(4-((2-(tert-butyl)-1H-imidazol-1-yl)methyl)phenyl)-2-propylthiazol-5-yl)sulfonyl) carbamate

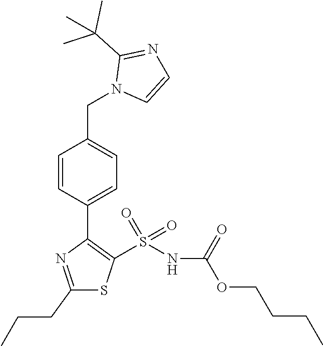 [SMILES: CCCCOC(=O)NS(=O)(=O)c1sc(CCC)nc1-c1ccc(Cn2ccnc2C(C)(C)C)cc1]

To a mixture of the corresponding sulfonamide (0.12 mmol, 1 equiv.) and triethylamine (0.60 mmol, 5 equiv.) in DCM (2 mL), butyl chloroformate (0.14 mmol, 1.2 equiv.) was added drop-wise over approximately 2 min and the reaction mixture was stirred at room temperature for 20 min. The reaction mixture was quenched with water and diluted with DCM (25 mL), then extracted with 10° A) citric acid (aq., 20 mL), water (3×30 mL) and brine (20 mL) in sequence. The combined organic layer was dried over MgSO₄ and concentrated. The crude product was purified by manual FCC (0-5% with MeOH in acetonitrile) and the product was obtained as white amorphous solid (43 mg, 69% yield). ¹H NMR (400 MHz, Acetone-d₆) δ 8.26 (d, J=8.5 Hz, 2H), 7.10 (d, J=8.5 Hz, 2H), 7.05 (d, J=1.5 Hz, 1H), 6.97 (d, J=1.5 Hz, 1H), 5.86 (s, 1H), 5.54 (s, 2H), 3.84 (t, J=6.6 Hz, 2H), 2.92 (t, J=7.6 Hz, 2H), 1.88-1.76 (m, 2H), 1.48-1.43 (m, 11H), 1.32-1.23 (m, 2H), 1.03 (t, J=7.4 Hz, 3H), 0.86 (t, J=7.4 Hz, 3H). ¹³C NMR (101 MHz, Acetone-d₆) δ 169.5, 160.0, 153.5, 150.6, 137.7, 137.6, 133.9, 130.4, 125.6, 124.4, 122.6, 64.4, 50.7, 34.7, 33.4, 31.1, 29.2, 22.8, 19.0, 13.3, 13.1. HRMS (ESI): calcd. for C₂₅H₃₅N₄O₄S₂⁺ [M+H]⁺ 519.2094; found: 519.2096.

### EXAMPLE 7A

### Butyl ((4′-((2-(2-hydroxypropan-2-yl)-1H-imidazol-1-yl)methyl)-5-isobutyl-[1,1′-biphenyl]-2-yl)sulfonyl)carbamate

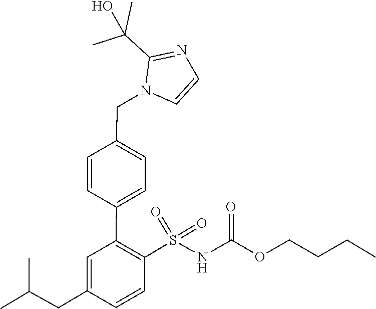 [SMILES: CCCCOC(=O)NS(=O)(=O)c1ccc(CC(C)C)cc1-c1ccc(Cn2ccnc2C(C)(C)O)cc1]

To a stirred solution of the corresponding sulfonamide (74.3 mg, 0.156 mmol) and triethyl amine (108.6 μL, 0.78 mmol, 5 equiv.) in DCM (1.0 mL) at room temperature was added butyl chloroformate (27.5 μL, 0.216 mmol). After 20 min the reaction was quenched with water (2 mL). The crude product was extracted with DCM (3×5 mL), washed with brine (5 mL), dried over anhydrous MgSO₄ and concentrated. The crude product was purified by FCC (5% MeOH in DCM) to afford the product as a white amorphous solid in 47% yield (39 mg, 73.5 μmol). ¹H NMR (400 MHz, Acetone-d₆) δ 8.11 (d, J=8.2 Hz, 1H), 7.43 (dd, J=8.2, 1.8 Hz, 1H), 7.38-7.29 (m, 2H), 7.29-7.23 (m, 2H), 7.14 (d, J=1.8 Hz, 1H), 6.89 (d, J=1.3 Hz, 1H), 6.77 (d, J=1.3 Hz, 1H), 5.65 (s, 2H), 3.96 (t, J=6.5 Hz, 2H), 2.62 (d, J=7.2 Hz, 2H), 2.01-1.88 (m, 1H), 1.63 (s, 6H), 1.51-1.39 (m, 2H), 1.32-1.15 (m, 2H), 0.93 (d, J=6.6 Hz, 6H), 0.85 (t, J=7.4 Hz, 3H). ¹³C NMR (101 MHz, Acetone-d₆) δ 153.0, 151.8, 148.1, 141.6, 139.5, 138.7, 136.6, 134.1, 131.1, 130.2, 129.1, 127.7, 126.7, 122.1, 71.1, 66.4, 50.9, 45.3, 31.3, 31.0, 30.8, 22.5, 19.5, 13.9. HRMS (ESI) calcd. for C₂₈H₃₈N₃O₅S⁺ [M+H]⁺ 528.2532 found 528.2526.

### EXAMPLE 7B

### Potassium (butoxycarbonyl)((4′-((2-(2-hydroxypropan-2-yl)-1H-imidazol-1-yl)methyl)-5-isobutyl-[1,1′-biphenyl]-2-yl)sulfonyl)amide

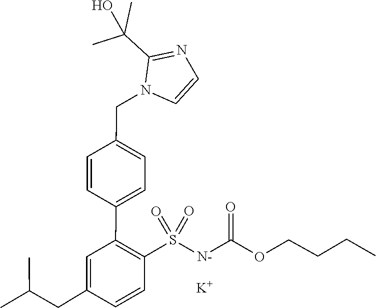 [SMILES: CCCCOC(=O)[N-]S(=O)(=O)c1ccc(CC(C)C)cc1-c1ccc(Cn2ccnc2C(C)(C)O)cc1 ; [K+]]

To a stirred solution of butyl ((4′-((2-(2-hydroxypropan-2-yl)-1H-imidazol-1-yl)methyl)-5-isobutyl-[1,1′-biphenyl]-2-yl)sulfonyl)carbamate (39 mg, 73.9 μmol) in chloroform was added KOH (4.47 mg, 79.8 μmol) in MeOH. The resulting solution was stirred at rt for 4 h, during which period precipitation of white solids was observed. The crude product was partially evaporated and triturated from chloroform with pentane (50 mL), The resulting was solids were filtered, then dissolved in water (5 mL). The solution was filtered to ensure removal of organic residues. The product was obtained as a white amorphous solid in 99% yield (41.5 mg, 73.4 μmol). ¹1H NMR (400 MHz, DMSO-d₆) δ 7.86 (d, J=8.1 Hz, 1H), 7.40 (d, J=8.1 Hz, 2H), 7.11 (t, J=8.1 Hz, 3H), 6.93 (d, J=1.3 Hz, 1H), 6.85 (d, J=1.8 Hz, 1H), 6.74 (d, J=1.2 Hz, 1H), 5.53 (s, 2H),5.42 (s, 1H), 3.54 (t, J=6.5 Hz, 2H), 2.49-2.42 (m, 2H), 1.90-1.73 (m, 1H), 1.52 (s, 6H), 1.39-1.26 (m, 2H), 1.27-1.13 (m, 2H), 0.87 (d, J=6.6 Hz, 6H), 0.83 (t, 3=7.3 Hz, 3H).

### Example 8A

### Methyl ((4′-((2-(2-hydroxypropan-2-yl)-1H-imidazol-1-yl)methyl)-5-isobutyl-[1,1′-biphenyl]-2-yl)sulfonyl)carbamate

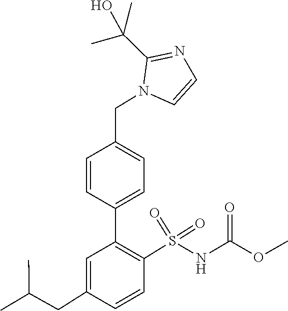 [SMILES: COC(=O)NS(=O)(=O)c1ccc(CC(C)C)cc1-c1ccc(Cn2ccnc2C(C)(C)O)cc1]

Butyl ((4′-((2-(2-hydroxypropan-2-yl)-1H-imidazol-1-yl)methyl)-5-isobutyl-[1,1′-biphenyl]-2-yl)sulfonyl)carbamate (16.2 mg, 30.7 μmol) was stirred in MeOH (1 mL) at 120 degrees (7 bar pressure) for 20 min under MW irradiation. The crude product was purified by FCC (2-4% MeOH in DCM) to afford the product as a white amorphous solid (14.7 mg, 99% yield). ¹1H NMR (400 MHz, Acetone-d₆) δ 8.12 (d, J=8.2 Hz, 1H), 7.42(dd, J=8.2, 1.8 Hz, 1H), 7.39-7.30 (m, 2H), 7.25 (d, J=8.2 Hz, 2H), 7.13 (d, J=1.8 Hz, 1H), 6.90 (d, J=1.3 Hz, 1H), 6.77 (d, J=1.3 Hz, 1H), 5.89 (s, 1H), 5.65 (s, 2H), 3.54 (s, 3H), 2.61 (d, J=7.2 Hz, 2H), 2.02-1.84 (m, 1H), 1.63 (s, 6H), 0.92 (d, J=6.6 Hz, 6H). ¹³C NMR (101 MHz, Acetone-d₆) δ 153.0, 152.6, 148.0, 141.6, 139.6, 138.6, 136.6, 134.0, 131.2, 130.2, 129.0, 127.7, 126.5, 122.2, 71.1, 53.1, 50.9, 45.3, 30.9, 30.7, 22.5. HRMS (ESU⁺) calcd. for C₂₅H₃₂N₃O₅S⁺ [M+H]⁺486.2063 found 486.2060.

### EXAMPLE 8B

### Potassium ((4′-((2-(2-hydroxypropan-2-yl)-1H-imidazol-1-yl)methyl)-5-isobutyl-[1,1′-biphenyl]-2-yl)sulfonyl)(methoxycarbonyl)amide

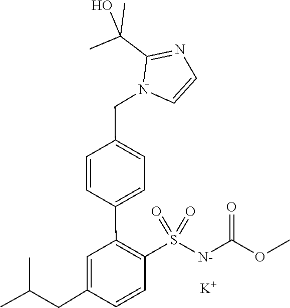 [SMILES: COC(=O)[N-]S(=O)(=O)c1ccc(CC(C)C)cc1-c1ccc(Cn2ccnc2C(C)(C)O)cc1 ; [K+]]

The title compound was synthesised as described for potassium (butoxycarbonyl)((4′-((2-(2-hydroxypropan-2-yl)-1Himidazol-1-yl)methyl)-5-isobutyl-[1,1′-biphenyl]-2-yl)sulfonyl)amide using methyl ((4′-((2-(2-hydroxypropan-2-yl)-1H-imidazol-1-yl)methyl)-5-isobutyl-[1,1′-biphenyl]-2-yl)sulfonyl)carbamate (14.7 mg, 30.2 μmol) and KOH (1.83 mg, 32.7 μmol). The product was obtained as a white amorphous solid (15.7 mg, 99% yield). ¹1H NMR (400 MHz, DMSO-d₆) δ 7.86 (d, J=8.0 Hz, 1H), 7.42 (d, J=8.2 Hz, 2H), 7.16-7.06 (m, 3H), 6.94 (d, J=1.3 Hz, 1H), 6.86 (d, J=1.8 Hz, 1H), 6.74 (d, J=1.3 Hz, 1H), 5.53 (s, 2H), 5.43 (s, 1H), 3.15 (s, 3H), 2.46 (d, J=7.1 Hz, 2H), 2.02-1.74 (m, 1H), 1.52 (s, 6H), 0.87 (d, J=6.6 Hz, 6H).

### EXAMPLE 9A

### Butyl ((4′-((2-(tert-butyl)-1H-imidazol-1-yl)methyl)-5-propyl-[1,1′-biphenyl]-2-yl)sulfonyl)carbamate

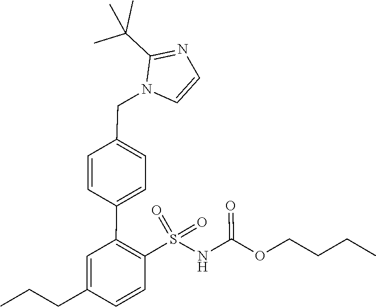 [SMILES: CCCCOC(=O)NS(=O)(=O)c1ccc(CCC)cc1-c1ccc(Cn2ccnc2C(C)(C)C)cc1]

The title compound was synthesized as described for butyl ((4′-((2-(2-hydroxypropan-2-yl)-1H-imidazol-1-yl)methyl)-5-isobutyl-[1,1′-biphenyl]-2-yl)sulfonyl)carbamate using the corresponding sulfonamide (107 mg, 0.261 mmol) and butyl chloroformate (45.8 μL, 0.360 mmol). The product was obtained as a white amorphous solid (48 mg, 36%). ¹H NMR (400 MHz, Acetone-d₆) δ 8.14 (d, J=8.2 Hz, 1H), 7.46 (dd, J=8.2, 1.9 Hz, 1H), 7.44-7.36 (m, 2H), 7.26-7.11 (m, 3H), 6.92 (d, J=1.4 Hz, 1H), 6.83 (d, J=1.4 Hz, 1H), 5.50 (s, 2H), 3.97 (t, J=6.5 Hz, 2H), 2.80-2.64 (m, 2H), 1.80-1.66 (m, 2H), 1.53-1.44 (m, 2H), 1.44 (s, 9H), 1.30-1.18 (m, 2H), 0.97 (t, J=7.3 Hz, 3H), 0.87 (t, J=7.4 Hz, 3H). ¹³C NMR (101 MHz, Acetone-d₆) δ 154.5, 152.2, 148.9, 141.5, 139.8, 138.2, 136.8, 133.4, 131.2, 130.3, 128.4, 126.9, 126.2, 122.8, 66.2, 51.3, 38.0, 34.1, 31.3, 30.3, 24.9, 19.5, 14.0, 13.9. HRMS (ESU⁺) calcd. for C₂₈H₃₈N₃O₄S⁺ [M+H]⁺: 512.2583; found: 512.2577.

### EXAMPLE 9B

### Potassium (butoxycarbonyl)((4′-((2-(tert-butyl)-1H-imidazol-1-yl)methyl)-5-propyl-[1,1′-biphenyl]-2-yl)sulfonyl)amide

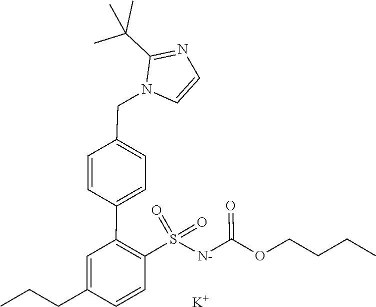 [SMILES: CCCCOC(=O)[N-]S(=O)(=O)c1ccc(CCC)cc1-c1ccc(Cn2ccnc2C(C)(C)C)cc1 ; [K+]]

The title compound was synthesised as described for potassium (butoxycarbonyl)((4′-((2-(2-hydroxypropan-2-yl)-1Himidazol-1-yl)methyl)-5-isobutyl-[1,1′-biphenyl]-2-yl)sulfonyl)amide using butyl ((4′-((2-(tert-butyl)-1H-imidazol-1-yl)methyl)-5-propyl-[1,1′-biphenyl]-2-yl)sulfonyl)carbamate (18.8 mg, 36.4 μmol) and KOH (2.20 mg, 39.3 μmol). ¹H NMR (400 MHz, DMSO-d₆) δ 7.85 (d, J=8.1 Hz, 1H), 7.41 (d, J=8.2 Hz, 2H), 7.16 (dd, J=8.1, 1.8 Hz, 1H), 6.96-6.92 (m, 3H), 6.88 (d, J=1.8 Hz, 1H), 6.76 (d, J=1.3 Hz, 1H), 5.37 (s, 2H), 3.54 (t, J=6.5 Hz, 2H), 2.60-2.52 (m, 2H), 1.73-1.49 (m, 2H), 1.36-1.30 (m, 2H), 1.34 (s, 9H), 1.25-1.11 (m, 2H), 0.89 (t, J=7.3 Hz, 3H), 0.83 (t, J=7.3 Hz, 3H).

### EXAMPLE 10A

### Methyl ((4′-((2-(tert-butyl)-1H-imidazol-1-yl)methyl)-5-propyl-[1,1′-biphenyl]-2-yl)sulfonyl)carbamate

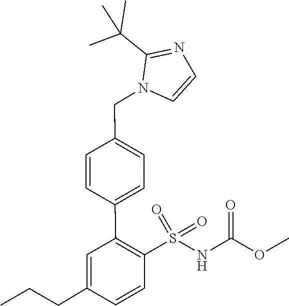 [SMILES: CCCc1ccc(S(=O)(=O)NC(=O)OC)c(-c2ccc(Cn3ccnc3C(C)(C)C)cc2)c1]

The title compound was synthesised as described for methyl ((4′-((2-(2-hydroxypropan-2-yl)-1H-imidazol-1-yl)methyl)-5-isobutyl-[1,1′-biphenyl]-2-yl)sulfonyl)carbamate using butyl ((4′-((2-(tert-butyl)-1H-imidazol-1-yl)methyl)-5-propyl-[1,1′-biphenyl]-2-yl)sulfonyl)carbamate (18.8 mg, 33.3_μmol). The crude product was concentrated and purified by FCC (4-6% MeOH in CH₂C₁₂) to afford the product as a white amorphous solid (41.5 mg, 86% yield). ¹H NMR (400 MHz, Acetone-d₆) δ 8.12 (d, J=8.2 Hz, 1H), 7.49 (s, br., 1H), 7.44 (dd, J=8.2, 1.9 Hz, 1H), 7.42-7.36 (m, 2H), 7.21-7.04 (m, 3H), 6.91 (d, J=1.4 Hz, 1H), 6.83 (d, J=1.4 Hz, 1H), 5.49 (s, 2H), 3.53 (s, 3H), 2.71 (dd, J=8.6, 6.7 Hz, 2H), 1.81-1.60 (m, 2H), 1.42 (s, 9H), 0.95 (t, J=7.3 Hz, 3H). ¹³C NMR (101 MHz, Acetone-d₆) δ 154.5, 152.9, 148.9, 141.5, 139.9, 138.1, 136.8, 133.3, 131.3, 130.3, 128.4, 126.9, 126.1, 122.9, 53.0, 51.3, 38.1, 34.1, 30.2, 24.9, 14.0. HRMS (ESI⁺) calcd. for C₂₅H₃₂N₃O₄S⁺ [M+H]⁺: 470.2114; found: 470.2115.

### EXAMPLE 10B

### Potassium ((4′-((2-(tert-butyl)-1H-imidazol-1-yl)methyl)-5-propyl-[1,1′-biphenyl]-2-yl)sulfonyl)(methoxycarbonyl)amide

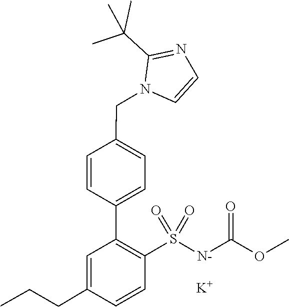 [SMILES: CCCc1ccc(S(=O)(=O)[N-]C(=O)OC)c(-c2ccc(Cn3ccnc3C(C)(C)C)cc2)c1 ; [K+]]

The title compound was synthesised as described for potassium (butoxycarbonyl)((4′-((2-(2-hydroxypropan-2-yl)-1Himidazol-1-yl)methyl)-5-isobutyl-[1,1′-biphenyl]-2-yl)sulfonyl)amide using Methyl ((4′-((2-(tert-butyl)-1H-imidazol-1-yl)methyl)-5-propyl-[1,1′-biphenyl]-2-yl)sulfonyl)carbamate (42.4 mg, 89.4 μmol) and KOH (5.41 mg, 96.5 μmol). The product was obtained as a white amorphous solid (42.2 mg, 93% yield). ¹1H NMR (400 MHz, DMSO-d₆) δ 7.86 (d, J=8.1 Hz, 1H), 7.43 (d, J=8.2 Hz, 2H), 7.16 (dd, J=8.1, 1.9 Hz, 1H), 7.05-6.91 (m, 3H), 6.89 (d, J=1.8 Hz, 1H), 6.77 (d, J=1.2 Hz, 1H), 5.38 (s, 2H), 3.15 (s, 3H), 2.58-2.52 (m, 2H), 1.66-1.52 (m, 2H), 1.34 (s, 9H), 0.90 (t, J=7.3 Hz, 3H).

### Example 11A

### Butyl ((3-(4-((2-(tert-butyl)-1H-imidazol-1-yl)methyl)phenyl)-5-isobutylthiophen-2-yl)sulfonyl)carbamate

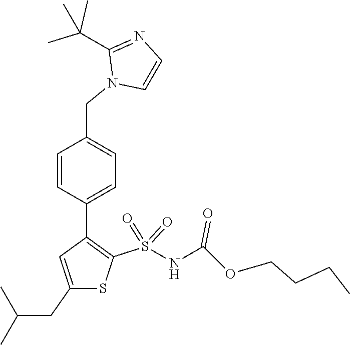 [SMILES: CCCCOC(=O)NS(=O)(=O)c1sc(CC(C)C)cc1-c1ccc(Cn2ccnc2C(C)(C)C)cc1]

The title compound was synthesized as described for butyl ((4′-((2-(2-hydroxypropan-2-yl)-1H-imidazol-1-yl)methyl)-5-isobutyl-[1,1′-biphenyl]-2-yl)sulfonyl)carbamate using the corresponding sulfonamide (168 mg, 0.389 mmol) and butyl chloroformate (68.3 μL, 0.537 mmol). The crude product was purified by FCC (5% MeOH in DCM) to afford the product as a white amorphous solid (179 mg, 87%). ¹H NMR (400 MHz, Acetone-d₆) δ 7.79-7.66 (m, 2H), 7.12 (d, J=8.1 Hz, 2H), 7.03 (d, J=1.5 Hz, 1H), 6.95-6.79 (m, 2H), 5.54 (s, 2H), 2.72 (d, J=7.1 Hz, 2H), 2.00-1.87 (m, 6.7 Hz, 1H), 1.54-1.44 (m, 2H), 1.43 (s, 9H), 1.32-1.18 (m, 4H), 0.99 (d, J=6.6 Hz, 6H), 0.86 (t, J=7.4 Hz, 3H). ¹³C NMR (101 MHz, Acetone-d₆) δ 154.3, 154.1, 149.4, 144.0, 137.8, 136.0, 135.3, 130.5, 130.0, 127.1, 124.5, 123.4, 65.8, 51.6, 39.5, 34.3, 31.5, 31.2, 29.9, 22.5, 19.6, 14.0. HRMS (ESU⁺) calcd. for C₂₇H₃₈N₃O₄S₂⁺ [M+H]⁺ 532.2304 found 532.2300.

### EXAMPLE 11B

### Potassium (butoxycarbonyl)((3-(4-((2-(tert-butyl)-1H-imidazol-1-yl)methyl)phenyl)-5-isobutylthiophen-2-yl)sulfonyl)amide

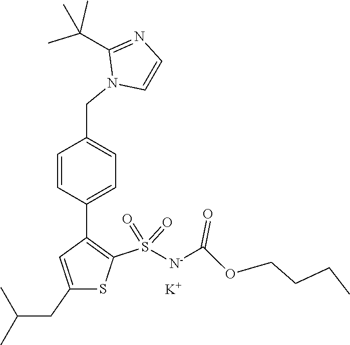 [SMILES: CCCCOC(=O)[N-]S(=O)(=O)c1sc(CC(C)C)cc1-c1ccc(Cn2ccnc2C(C)(C)C)cc1 ; [K+]]

The title compound was synthesised as describe for potassium (butoxycarbonyl)((4′-((2-(2-hydroxypropan-2-yl)-1H-imidazol-1-yl)methyl)-5-isobutyl-[1,1′-biphenyl]-2-yl)sulfonyl)amide using butyl ((3-(4-((2-(tert-butyl)-1H-imidazol-1-yl)methyl)phenyl)-5-isobutylthiophen-2-yl)sulfonyl)carbamate (33.1 mg, 67 μmol, 1,0 equiv.) and KOH (4.06 mg, 72.3 μmol, 1.08 equiv.) in MeOH (0.59 M). The product was obtained as a white amorphous solid (29.7 mg, 87% yield). ¹1H NMR (400 MHz, DMSO-d₆) δ 7.77 (d, J=8.3 Hz, 2H), 7.03-6.89 (m, 3H), 6.86-6.69 (m, 2H), 5.37 (s, 2H), 3.23 (s, 3H), 2.60 (d, J=6.9 Hz, 6H), 1.91-1.70 (m, 1H), 1.31 (s, 9H), 0.93 (d, J=6.6 Hz, 6H).

### EXAMPLE 12A

### Methyl ((3-(4-((2-(tert-butyl)-1H-imidazol-1-yl)methyl)phenyl)-5-isobutylthiophen yl)sulfonyl)carbamate

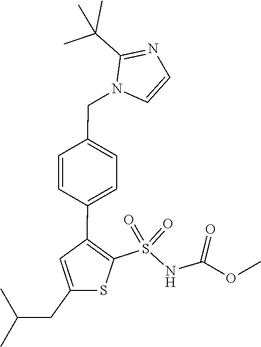 [SMILES: COC(=O)NS(=O)(=O)c1sc(CC(C)C)cc1-c1ccc(Cn2ccnc2C(C)(C)C)cc1]

Butyl ((3-(4-((2-(tert-butyl)-1H-imidazol-1-yl)methyl)phenyl)-5-isobutylthiophen-2-yl)sulfonyl)carbamate (45.8 mg, 86.1 μmol) was stirred in MeOH (1 mL) at 120° C. (7 bar pressure) for 20 min under MWI. The crude product was concentrated and purified by FCC (5% MeOH in DCM) to afford the product as a white amorphous solid (35.7 mg, 85% yield). ¹H NMR (400 MHz, Methanol-c/a) δ 7.65 (d, J=8.3 Hz, 2H), 7.21-7.03 (m, 4H), 6.74 (s, 1H), 5.51 (s, 2H), 3.47 (s, 3H), 2.73-2.61 (m, 2H), 1.97-1.83 (m, 1H), 1.50 (s, 9H), 0.97 (d, J=6.6 Hz, 6H). ¹³C NMR (101 MHz, Methanol-d₄) δ 158.5, 154.0, 148.5, 143.1, 136.9, 136.3, 135.2, 130.8, 129.5, 127.2, 124.3, 121.2, 52.7, 52.4, 39.7, 34.3, 31.2, 29.2, 22.5. HRMS (ESU⁺) calcd. for C₂₄H₃₃N₃O₄S₂⁺ [M+H]⁺ 490.1834 found 490.1838.

### EXAMPLE 12B

### Potassium ((3-(4-((2-(tert-butyl)-1H-imidazol-1-yl)methyl)phenyl)-5-isobutylthiophen-2-yl)sulfonyl)(methoxycarbonyl)amide

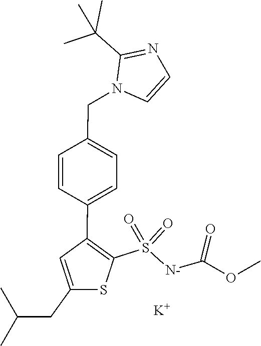 [SMILES: COC(=O)[N-]S(=O)(=O)c1sc(CC(C)C)cc1-c1ccc(Cn2ccnc2C(C)(C)C)cc1 ; [K+]]

The title compound was synthesized as described for potassium (butoxycarbonyl)((4′-((2-(2-hydroxypropan-2-yl)-1H-imidazol-1-yl)methyl)-5-isobutyl-[1,1′-biphenyl]-2-yl)sulfonyl)amide using methyl ((3-(4-((2-(tert-butyl)-1H-imidazol-1-yl)methyl)phenyl)-5-isobutylthiophen-2-yl)sulfonyl)carbamate (33.1 mg, 67 μmol, 1.0 equiv.) and KOH (4.06 mg, 72.3 μmol, 1.08 eguiv,) in MeOH (0.59 M). The product was obtained as a white amorphous solid (29.7 ma, 87% yield). ¹1H NMR (400 MHz, DMSO-d₆) δ 7.77 (d, J=8.3 Hz, 2H), 7.03-6.89 (m, 3H), 6.86-6.69 (m, 2H), 5.37 (s, 2H), 3.23 (s, 3H), 2.60 (d, J=6.9 Hz, 6H), 1.91-1.70 (m, 1H), 1.31 (s, 9H), 0.93 (d, J=6.6 Hz, 6H).

### EXAMPLE 13

### Sodium ((3-(4-((1H-imidazol-1-yl)methyl)phenyl)-5-isobutylthiophen-2-yl)sulfonyl)(butoxycarbonyl)amide

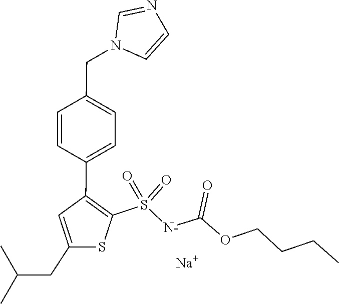 [SMILES: CCCCOC(=O)[N-]S(=O)(=O)c1sc(CC(C)C)cc1-c1ccc(Cn2ccnc2)cc1 ; [Na+]]

A solution of butyl ((3-(4-((1H-imidazol-1-yl)methyl)phenyl)-5-isobutylthiophen-2-yl)sulfonyl)carbamate (100 g, 0.210 mol) in dichloromethane (380 mL) was filtered through a Celite bead. To the filtrate was added 3.3% NaOH solution in methanol (198 g) at 25-30° C. and maintained under stirring at the same temperature for 1 h. The above solution was concentrated to get approximately 200 mL and the residue was cooled to 25-30° C. and 1.46 L of isopropyl was added. The resultant solution was concentrated to get approximately 400 mL of oily syrup. Heptane (1.18 I) was added to the oily syrup slowly under stirring. The solids obtained were filtered and washed with heptane (200 mL). The solid salt were dried below 50° C. for 8 h to get 85% yield (89 g, 0.178 mol) of sodium ((3-(4-((1H-imidazol-1-yl)methyl)phenyl)-5-isobutylthiophen-2-yl)sulfonyl)(butoxycarbonyl)amide (C21) as a white solid with HPLC purity >98%.

### EXAMPLE 14

### Butyl ((4-(4-((2-(2-hydroxypropan-2-yl)-1H-imidazol-1-yl)methyl)phenyl)-2-propyl thiazol-5-yl)sulfonyl)carbamate

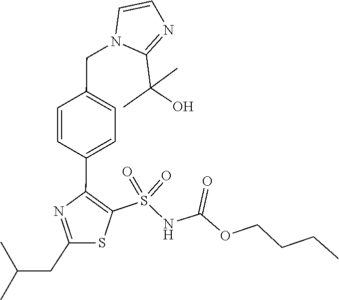 [SMILES: CCCCOC(=O)NS(=O)(=O)c1sc(CC(C)C)nc1-c1ccc(Cn2ccnc2C(C)(C)O)cc1]

The title compound was synthesized as described for methyl((4-(4-((2-(tert-butyl)-1H-imidazol-1-yl)methyl)phenyl)-2-isobutylthiazol-5-yl)sulfonyl) carbamate using the corresponding sulfonamide (0.16 mmol, 1 equiv.) and triethylamine (0.8 mmol, 5 equiv.) except that the mixture was kept over dry ice, wherein butylchloroformate (0.2 mmol, 1.2 equiv.) was added drop-wise over approximately 2 min and the reaction mixture was stirred over dry ice for 10 min. The crude product was purified by manual FCC (0-5% with MeOH in acetonitrile) and the product was obtained as white amorphous solid (48 mg, 55% yield). ¹H NMR (400 MHz, Methanol-d₄) δ 7.81 (d, J=8.4 Hz, 2H), 7.27-7.16 (m, 4H), 5.63 (s, 2H), 3.78 (t, J=6.5 Hz, 2H), 2.89 (dd, J=8.0, 7.2 Hz, 2H), 1.80-1.71 (m, 2H), 1.58 (s, 6H), 1.41-1.32 (m, 2H), 1.20-1.14 (m, 2H), 0.95 (t, J=7.4 Hz, 3H), 0.77 (t, J=7.4 Hz, 3H). ¹³C NMR (101 MHz, Methanol-d₄) δ 173.1, 157.2, 152.9, 151.7, 136.3, 133.5, 135.0, 130.1, 126.9, 123.3, 120.1, 69.3, 64.8, 51.2, 34.6, 30.7, 28.4, 22.9, 18.7, 12.7, 12.5. HRMS (ESI): calcd. for C₂₄H₃₃N₄O₂⁺ [M+H]⁺ 521.1887; found: 521.1879.

### EXAMPLE 15

### Butyl ((4-(4-((2-isopropyl-1H-imidazol-1-yl)methyl)phenyl)-2-propylthiazol-5-yl) sulfonyl)carbamate

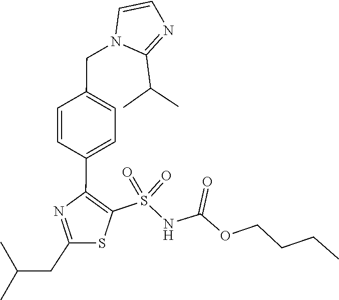 [SMILES: CCCCOC(=O)NS(=O)(=O)c1sc(CC(C)C)nc1-c1ccc(Cn2ccnc2C(C)C)cc1]

The title compound was synthesized as described for methyl((4-(4-((2-(tert-butyl)-1H-imidazol-1-yl)methyl)phenyl)-2-isobutylthiazol-5-yl)sulfonyl) carbamate using the corresponding sulfonamide (0.17 mmol, 1 equiv.) and triethylamine (0.86 mmol, 5 equiv.) except that the mixture was kept over dry ice, wherein butylchloroformate (0.2 mmol, 1.2 equiv.) was added drop-wise over approximately 2 min and the reaction mixture was stirred over dry ice for 10 min. The crude product was purified by manual FCC (0-5% with MeOH in acetonitrile) and the product was obtained as white amorphous solid (51 mg, 58% yield). ¹1H NMR (400 MHz, Acetone-d₆) δ 8.32-8.19 (m, 2H), 7.8-7.16 (m, 3H), 7.01 (d, J=1.5 Hz, 1H), 5.34 (s, 2H), 3.83 (t, J=6.7 Hz, 2H), 3.26-3.18 (m, 1H), 2.97-2.85 (m, 2H), 1.80 (dt, J=15.3, 7.6 Hz, 2H), 1.56-1.38 (m, 2H), 1.37-1.17 (m, 8H), 1.02 (t, J=7.4 Hz, 3H), 0.86 (t, J=7.4 Hz, 3H). ¹³C NMR (101 MHz, Acetone-d₆) δ 169.4, 160.2, 152.6, 150.5, 137.9, 137.1, 134.1, 130.4, 126.0, 125.1, 120.1, 64.3, 48.9, 34.7, 31.1, 25.6, 22.8, 21.1, 19.0, 13.3, 13.1. HRMS (ESI): calcd. for C₂₄H₃₃N₄O₄S₂⁺ [M+H]⁺ 505.1938; found: 505.1930.

### EXAMPLE 16

### Butyl ((2-isobutyl-4-(4-((2-(thiazol-2-yl)-1H-imidazol-1-yl)methyl)phenyl)thiazol-5-yl) su Ifonyl)carbamate

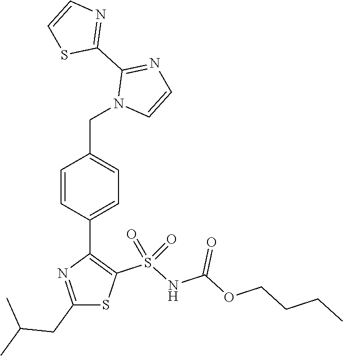 [SMILES: CCCCOC(=O)NS(=O)(=O)c1sc(CC(C)C)nc1-c1ccc(Cn2ccnc2-c2nccs2)cc1]

The title compound was synthesized as described for methyl((4-(4-((2-(tert-butyl)-1H-imidazol-1-yl)methyl)phenyl)-2-isobutylthiazol-5-yl)sulfonyl) carbamate using the corresponding sulfonamide (0.11 mmol, 1 equiv.) and triethylamine (0.54 mmol, 5 equiv.) except that the mixture was kept over dry ice, wherein butylchloroformate (0.13 mmol, 1.2 equiv.) was added drop-wise over approximately 2 min and the reaction mixture was stirred over dry ice for 10 min. The crude product was purified by manual FCC (0-5% with MeOH in acetonitrile) and the product was obtained as white amorphous solid (31 mg, 51% yield). ¹1H NMR (400 MHz, Acetone-d₆) δ 7.97 (d, J=8.4 Hz, 2H), 7.89 (d, J=3.3 Hz, 1H), 7.62 (d, J=3.3 Hz, 1H), 7.37 (d, J=1.2 Hz, 1H), 7.35 (d, J=8.7 Hz, 2H), 7.11 (d, J=1.2 Hz, 1H), 6.02 (s, 2H), 3.97 (t, J=6.5 Hz, 2H), 2.92 (d, J=7.1 Hz, 2H), 2.16 (dt, J=13.5, 6.8 Hz, 1H), 1.52-1.42 (m, 2H), 1.30-1.19 (m, 2H), 1.03 (d, J=6.6 Hz, 6H), 0.86 (t, J=7.4 Hz, 3H). ¹³C NMR (101 MHz, Acetone-d₆) δ 172.3, 160.1, 154.5, 153.0, 143.3, 140.2, 138.9, 132.8, 132.3, 130.1, 129.5, 127.0, 123.7, 120.0, 65.7, 49.9, 41.7, 30.5, 29.4, 21.6, 18.7, 13.1. HRMS (ESI): calcd. for C₂₅H₃₀N₅O₄S₃⁺ [M+H]⁺560.1454; found: 560.1453.

### EXAMPLE 17

### Butyl ((4-(4-((2-(2-hydroxypropan-2-yl)-1H-imidazol-1-yl)methyl)phenyl)-2-isobutyl thiazol-5-yl)sulfonyl)carbamate

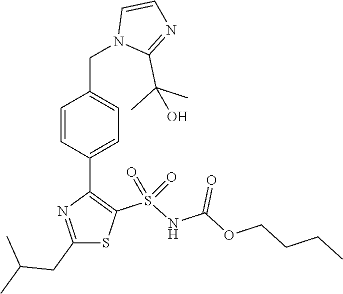 [SMILES: CCCCOC(=O)NS(=O)(=O)c1sc(CC(C)C)nc1-c1ccc(Cn2ccnc2C(C)(C)O)cc1]

The title compound was synthesized as described for methyl ((4-(4-((2-(tert-butyl)-1H-imidazol-1-yl)methyl)phenyl)-2-isobutylthiazol-5-yl)sulfonyl) carbamate using the corresponding sulfonamide (0.18 mmol, 1 equiv.) and triethylamine (0.9 mmol, 5 equiv.) except that the mixture was kept over dry ice, wherein butylchloroformate (0.22 mmol, 1.2 equiv.) was added drop-wise over approximately 2 min and the reaction mixture was stirred over dry ice for 10 min. The crude product was purified by manual FCC (0-5% with MeOH in acetonitrile) and the product was obtained as white amorphous solid (51 mg, 52° A) yield). ¹H NMR (400 MHz, Methanol-d₄) δ 7.94 (d, J=8.4 Hz, 2H), 7.37-7.28 (m, 4H), 5.75 (s, 2H), 3.90 (t, J=6.5 Hz, 2H), 2.90 (d, J=7.2 Hz, 2H), 2.18-2.11 (m, 1H), 1.70 (s, 6H), 1.55-1.44 (m, 2H), 1.34-1.23 (m, 2H), 1.05 (d, J=6.7 Hz, 6H), 0.89 (t, J=7.4 Hz, 3H). ¹³C NMR (101 MHz, Methanol-d₄) δ 172.0, 157.3, 152.8, 151.6, 136.3, 135.3, 133.5, 130.1, 126.9, 123.3, 120.1, 69.4, 64.8, 51.2, 41.5, 30.7, 29.6, 28.4, 21.2, 18.7, 12.7. HRMS (ESI): calcd. for C₁₉H₂₂N₄O₄S₂ [M+H]⁺ 435.1155; found: 435.1174. HRMS (ESI): calcd. for C₂₅H₃₅N₄O₅S₂⁺ [M+H]⁺ 535.2043; found: 535.2035.

### EXAMPLE 18

### Butyl ((2-isobutyl-4-(4-((2-isopropyl-1H-imidazol-1-yl)methyl)phenyl)thiazol-5-yl) sulfonyl)carbamate

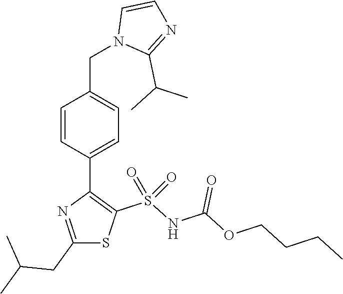 [SMILES: CCCCOC(=O)NS(=O)(=O)c1sc(CC(C)C)nc1-c1ccc(Cn2ccnc2C(C)C)cc1]

The title compound was synthesized as described for butyl ((4-(4-((2-(2-hydroxypropan-2-yl)-1H-imidazol-1-yl)methyl)phenyl)-2-propyl thiazol-5-yl)sulfonyl)carbamate using the corresponding sulfonamide (80.0 mg, 0.191 mmol) and butyl chloroformate (29.6 μL, 0.232 mmol). The crude product was purified by FCC (0-5% with MeOH in MeCN) to afford the product was as a white amorphous solid (51 mg, 51% yield). ¹H NMR (400 MHz, Acetone-d₆) δ 8.26 (d, J=8.4 Hz, 2H), 7.20-7.19 (m, 3H), 7.05 (d, J=1.4 Hz, 1H), 5.36 (s, 2H), 3.83 (t, J=6.7 Hz, 2H), 3.30-3.18 (m, 1H), 2.83 (d, J=7.1 Hz, 2H), 2.20-2.08 (m, 1H), 1.49-1.40 (m, 2H), 1.33-1.21 (m, 8H), 1.02 (d, J=6.6 Hz, 6H), 0.86 (t, J=7.4 Hz, 3H). ¹³C NMR (101 MHz, Acetone-d₆) δ 168.4, 160.01, 152.5, 150.5, 138.0, 136.9, 134.1, 130.6, 126.1, 124.8, 120.2, 64.3, 49.0, 41.6, 31.1, 29.4, 25.6, 21.7, 21.0, 18.9, 13.2. HRMS (ESI⁺): calcd. for C₂₅H₃₅N₄O₄S₂⁺ [M+H]⁺519.2094; found: 519.2084.

### EXAMPLE 19

### Methyl ((4-(4-((2-(2-hydroxypropan-2-yl)-1H-imidazol-1-yl)methyl)phenyl)-2-isobutylthiazol-5-yl)sulfonyl)carbamate

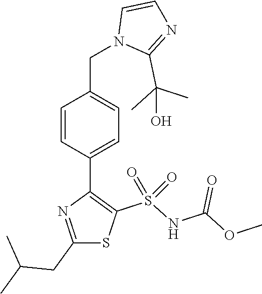 [SMILES: COC(=O)NS(=O)(=O)c1sc(CC(C)C)nc1-c1ccc(Cn2ccnc2C(C)(C)O)cc1]

The title compound was synthesized as described for methyl((4-(4-((2-(tert-butyl)-1H-imidazol-1-yl)methyl)phenyl)-2-isobutylthiazol-5-yl)sulfonyl) carbamate using the corresponding sulfonamide (0.13 mmol, 1 equiv.) and triethylamine (0.69 mmol, 5 equiv.) except that the mixture was kept over dry ice, wherein methylchloroformate (0.16 mmol, 1.2 equiv.) was added drop-wise over approximately 2 min and the reaction mixture was stirred over dry ice for 10 min. The crude product was purified by manual FCC (0-5% with MeOH in acetonitrile) and the product was obtained as white amorphous solid (41 mg, 60% yield). ¹1H NMR (400 MHz, Methanol-d₄) δ 7.81 (d, J=8.4 Hz, 2H), 7.28 (d, J=2.0 Hz, 1H), 7.27 (d, J=2.0 Hz, 1H), 7.23 (d, J=8.4 Hz, 2H), 5.64 (s, 2H), 3.37 (s, 3H), 2.79 (d, J=7.2 Hz, 2H), 2.08-1.97 (m, 1H), 1.58 (s, 6H), 0.93 (d, J=6.6 Hz, 6H). ¹³C NMR (101 MHz, Methanol-d₄) δ 172.2, 157.7, 152.9, 151.5, 135.9, 135.0, 133.7, 130.2, 126.9, 123.6, 119.3, 69.3, 51.5, 51.4, 41.4, 29.6, 28.2, 21.2. HRMS (ESI): calcd. for C₂₂H₂₉N₄O₅S₂⁺ [M+H]⁺ 493.1574; found: 493.1566.

### EXAMPLE 20

### Methyl ((2-isobutyl-4-(4-((2-isopropyl-1H-imidazol-1-yl)methyl)phenyl)thiazol-5-yl) sulfonyl)carbamate

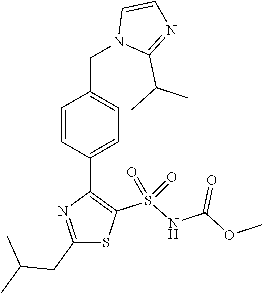 [SMILES: COC(=O)NS(=O)(=O)c1sc(CC(C)C)nc1-c1ccc(Cn2ccnc2C(C)C)cc1]

The title compound was synthesized as described for methyl((4-(4-((2-(tert-butyl)-1H-imidazol-1-yl)methyl)phenyl)-2-isobutylthiazol-5-yl)sulfonyl) carbamate using the corresponding sulfonamide (0.16 mmol, 1 equiv.) and triethylamine (0.83 mmol, 5 equiv.) except that the mixture was kept over dry ice, wherein methylchloroformate (0.2 mmol, 1.2 equiv.) was added drop-wise over approximately 2 min and the reaction mixture was stirred over dry ice for 10 min. The crude product was purified by manual FCC (0-5% with MeOH in acetonitrile) and the product was obtained as white amorphous solid (41 mg, 51% yield). ¹1H NMR (400 MHz, Methanol-d₄) δ 7.85 (d, J=8.4 Hz, 2H), 7.09 (d, J=8.6 Hz, 2H), 7.05 (d, J=1.6 Hz, 1H), 6.95 (d, J=1.6 Hz, 1H), 5.20 (s, 2H), 3.32 (s, 3H), 3.14-3.05 (m, 1H), 2.76 (d, J=7.2 Hz, 2H), 2.01 (dt, J=13.5, 6.8 Hz, 1H), 1.13 (d, J=6.9 Hz, 6H), 0.92 (d, J=6.6 Hz, 6H). ¹³C NMR (101 MHz, Methanol-d₄) δ 171.0, 160.5, 153.1, 151.8, 136.8, 136.5, 133.5, 130.2, 126.0, 124.0, 120.4, 51.1, 49.0, 41.4, 29.6, 25.5, 21.2, 20.4. HRMS (ESI): calcd. for C₂₂H₂₉N₄O₄S₂⁺ [M+H]⁺ 477.1625; found: 477.1618.

### EXAMPLE 21

### Butyl ((2-propyl-4-(4-((2-(thiazol-2-yl)-1H-imidazol-1-yl)methyl)phenyl)thiazol-5-yl) sulfonyl)carbamate

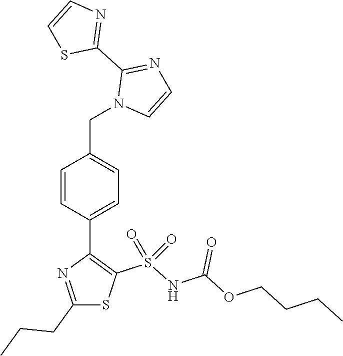 [SMILES: CCCCOC(=O)NS(=O)(=O)c1sc(CCC)nc1-c1ccc(Cn2ccnc2-c2nccs2)cc1]

The title compound was synthesized as described for methyl((4-(4-((2-(tert-butyl)-1H-imidazol-1-yl)methyl)phenyl)-2-isobutylthiazol-5-yl)sulfonyl) carbamate using the corresponding sulfonamide (0.11 mmol, 1 equiv.) and triethylamine (0.56 mmol, 5 equiv.) except that the mixture was kept over dry ice, wherein butylchloroformate (0.14 mmol, 1.2 equiv.) was added drop-wise over approximately 2 min and the reaction mixture was stirred over dry ice for 10 min. The crude product was purified by manual FCC (0-5% with MeOH in acetonitrile) and the product was obtained as white amorphous solid (37 mg, 60° A) yield). ¹H NMR (400 MHz, Acetone-d₆) δ 7.95 (d, J=8.3 Hz, 2H), 7.89 (d, J=3.3 Hz, 1H), 7.63 (d, J=3.3 Hz, 1H), 7.38 (d, J=1.2 Hz, 1H), 7.35 (d, J=8.7 Hz, 2H), 7.12 (d, J=1.2 Hz, 1H), 6.03 (s, 2H), 3.97 (t, J=6.5 Hz, 2H), 3.02 (t, J=7.5 Hz, 2H), 1.86 (h, J=7.4 Hz, 2H), 1.52-1.41 (m, 2H), 1.29-1.19 (m, 2H), 1.04 (t, J=7.4 Hz, 3H), 0.86 (t, J=7.4 Hz, 3H). ¹³C NMR (101 MHz, Acetone-d₆) 5 170.6, 160.2, 157.68, 151.9, 143.3, 140.2, 138.1, 135.8, 133.5, 130.2, 129.4, 126.8, 123.7, 119.9, 64.8, 50.0, 34.7, 30.9, 22.8, 18.8, 13.2, 13.0. HRMS (ESI): calcd. for C₂₄H₂₈N₅O₄S₃⁺ [M+H]⁺546.1298; found: 546.1295.

### EXAMPLE 22

### Butyl ((4-(4-((2-(1-hydroxyethyl)-1H-imidazol-1-yl)methyl)phenyl)-2-isobutylthiazol-5-yl)sulfonyl)carbamate

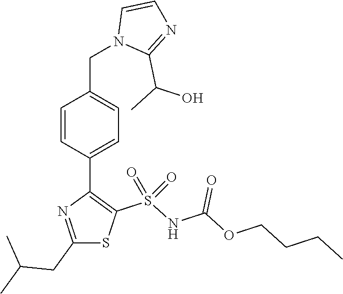 [SMILES: CCCCOC(=O)NS(=O)(=O)c1sc(CC(C)C)nc1-c1ccc(Cn2ccnc2C(C)O)cc1]

The title compound was synthesized as described for methyl((4-(4-((2-(tert-butyl)-1H-imidazol-1-yl)methyl)phenyl)-2-isobutylthiazol-5-yl)sulfonyl) carbamate using the corresponding sulfonamide (0.13 mmol, 1 equiv.) and triethylamine (0.68 mmol, 5 equiv.) except that the mixture was kept over dry ice, wherein butylchloroformate (0.16 mmol, 1.2 equiv.) was added drop-wise over approximately 2 min and the reaction mixture was stirred over dry ice for 10 min. The crude product was purified by manual FCC (0-5% with MeOH in acetonitrile) and the product was obtained as white amorphous solid (44 mg, 64° A) yield). ¹H NMR (400 MHz, Acetonitrile-d3) δ 8.04 (d, J=8.0 Hz, 2H), 7.19 (d, J=7.9 Hz, 2H), 7.06 (s, 1H), 6.99 (s, 1H), 5.42-5.24 (m, 2H), 5.00-4.96 (m, 1H), 3.83 (t, J=6.6 Hz, 2H), 2.85 (s, 2H), 2.11 (dt, J=13.5, 6.7 Hz, 1H), 1.55-1.39 (m, 5H), 1.32-1.20 (m, 2H), 1.02 (d, J=6.7 Hz, 6H), 0.88 (t, J=7.4 Hz, 3H). ¹³C NMR (101 MHz, Acetonitrile-d3) δ 170.2, 159.8, 151.5, 138.1, 137.3, 134.6, 130.7, 127.1, 125.2, 121.9, 121.7, 64.9, 62.4, 49.9, 42.1, 31.5, 30.0, 22.3, 22.1, 19.5, 13.7. HRMS (ESI): calcd. for C₂₄H₃₃N₄O₅S₂⁺ [M+H]⁺521.1887; found: 521.1891.

### EXAMPLE 23

### Methyl ((4-(4-((2-(1-hydroxyethyl)-1H-imidazol-1-yl)methyl)phenyl)-2-isobutylthiazol-5-yl)sulfonyl)carbamate

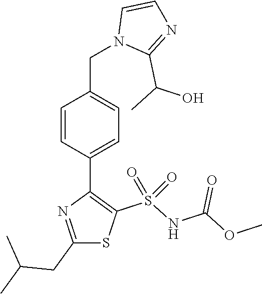 [SMILES: COC(=O)NS(=O)(=O)c1sc(CC(C)C)nc1-c1ccc(Cn2ccnc2C(C)O)cc1]

The title compound was synthesized as described for methyl((4-(4-((2-(tert-butyl)-1H-imidazol-1-yl)methyl)phenyl)-2-isobutylthiazol-5-yl)sulfonyl) carbamate using the corresponding sulfonamide (0.13 mmol, 1 equiv.) and triethylamine (0.65 mmol, 5 equiv.) except that the mixture was kept over dry ice, wherein methylchloroformate (0.16 mmol, 1.2 equiv.) was added drop-wise over approximately 2 min and the reaction mixture was stirred over dry ice for 10 min. The crude product was purified by manual FCC (0-5% with MeOH in acetonitrile) and the product was obtained as white amorphous solid (31 mg, 49° A) yield). ¹1H NMR (400 MHz, Acetone-d₆) δ 8.06 (d, J=8.4 Hz, 2H), 7.35 (d, J=1.7 Hz, 1H), 7.26 (d, J=8.1 Hz, 2H), 7.21 (d, J=1.7 Hz, 1H), 5.63 (s, 2H), 5.31 (q, J=6.7 Hz, 1H), 3.51 (s, 3H), 2.92 (d, J=7.1 Hz, 2H), 2.21-2.14 (m, 1H), 1.56 (d, J=6.7 Hz, 3H), 1.05 (d, J=6.7 Hz, 6H). ¹³C NMR (101 MHz, Acetone-d₆) δ 171.2, 156.0, 152.7, 150.2, 142.6, 136.2, 134.0, 130.3, 126.6, 122.2, 121.8, 61.8, 51.7, 50.0, 41.7, 29.4, 21.7, 21.6. HRMS (ESI): calcd. for C₂₁H₂₇N₄O₅S₂⁺ [M+H]⁺ 479.1417; found: 479.1418.

### EXAMPLE 24

### Butyl ((4-(4-((2-(1-hydroxyethyl)-1H-imidazol-1-yl)methyl)phenyl)-2-propylthiazol-5-yl)sulfonyl)carbamate

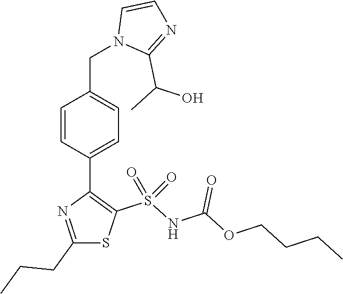 [SMILES: CCCCOC(=O)NS(=O)(=O)c1sc(CCC)nc1-c1ccc(Cn2ccnc2C(C)O)cc1]

The title compound was synthesized as described for methyl((4-(4-((2-(tert-butyl)-1H-imidazol-1-yl)methyl)phenyl)-2-isobutylthiazol-5-yl)sulfonyl) carbamate using the corresponding sulfonamide (0.14 mmol, 1 equiv.) and triethylamine (0.68 mmol, 5 equiv.) except that the mixture was kept over dry ice, wherein butylchloroformate (0.16 mmol, 1.2 equiv.) was added drop-wise over approximately 2 min and the reaction mixture was stirred over dry ice for 10 min. The crude product was purified by manual FCC (0-5% with MeOH in acetonitrile) and the product was obtained as white amorphous solid (38 mg, 55% yield). ¹1H NMR (400 MHz, Methanol-d₄) δ 7.95 (d, J=8.3 Hz, 2H), 7.41 (d, J=1.8 Hz, 1H), 7.39-7.29 (m, 3H), 5.50 (d, J=2.2 Hz, 2H), 5.13 (q, J=6.6 Hz, 1H), 3.88 (t, J=6.5 Hz, 2H), 3.13-2.92 (m, 2H), 1.97-1.79 (m, 2H), 1.56 (d, J=6.6 Hz, 3H), 1.50-1.44 (m, 2H), 1.33-1.26 (m, 2H), 1.07 (t, J=7.4 Hz, 3H), 0.89 (t, J=7.4 Hz, 3H). ¹³C NMR (101 MHz, Methanol-d₄) δ 172.7, 158.2, 152.3, 150.0, 135.8, 135.5, 133.8, 130.2, 126.4, 122.4, 120.5, 64.6, 61.1, 50.1, 34.6, 30.7, 23.0, 20.5, 18.7, 12.7, 12.5. HRMS (ESI): calcd. for C₂₃H₃₁N₄O₅S₂⁺ [M+H]⁺ 507.1730; found: 507.1735.

### EXAMPLE 25

### Methyl ((4-(4-((2-(1-hydroxyethyl)-1H-imidazol-1-yl)methyl)phenyl)-2-propylthiazol-5-yl)sulfonyl)carbamate

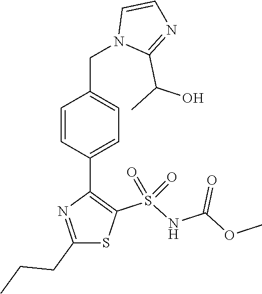 [SMILES: CCCc1nc(-c2ccc(Cn3ccnc3C(C)O)cc2)c(S(=O)(=O)NC(=O)OC)s1]

The title compound was synthesized as described for methyl((4-(4-((2-(tert-butyl)-1H-imidazol-1-yl)methyl)phenyl)-2-isobutylthiazol-5-yl)sulfonyl) carbamate using the corresponding sulfonamide (0.13 mmol, 1 equiv.) and triethylamine (0.68 mmol, 5 equiv.) except that the mixture was kept over dry ice, wherein methylchloroformate (0.16 mmol, 1.2 equiv.) was added drop-wise over approximately 2 min and the reaction mixture was stirred over dry ice for 10 min. The crude product was purified by manual FCC (0-5% with MeOH in acetonitrile) and the product was obtained as white amorphous solid (31 mg, 49% yield). ¹H NMR (400 MHz, DMSO-d₆) δ 8.13 (d, J=8.1 Hz, 2H), 7.63 (d, J=1.8 Hz, 1H), 7.52 (d, J=1.8 Hz, 1H), 7.30 (d, J=8.1 Hz, 2H), 6.21 (s, 1H), 5.48 (s, 2H), 5.16 (q, J=6.6 Hz, 1H), 3.31 (s, 3H), 2.90 (t, J=7.5 Hz, 2H), 1.75 (q, J=7.4 Hz, 2H), 1.44 (d, J=6.5 Hz, 3H), 0.98 (t, J=7.3 Hz, 3H). ¹³C NMR (101 MHz, DMSO-d₆) δ 170.1, 163.5, 158.7, 150.1, 149.8, 135.5, 134.3, 130.6, 127.3, 123.0, 121.0, 60.7, 51.7, 50.1, 34.8, 23.0, 22.2, 13.9. HRMS (ESI): calcd. for C₂₀H₂₅N₄O₅S₂⁺ [M+H]⁺ 465.1261; found: 465.1267.

### EXAMPLE 26

### Methyl ((4-(4-((2-cyclopropyl-1H-imidazol-1-yl)methyl)phenyl)-2-isobutylthiazol-5-yl)sulfonyl)carbamate

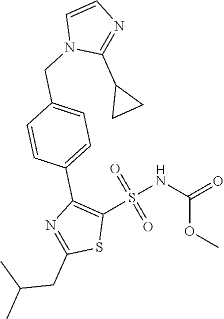 [SMILES: COC(=O)NS(=O)(=O)c1sc(CC(C)C)nc1-c1ccc(Cn2ccnc2C2CC2)cc1]

The title compound was synthesized as described for methyl((4-(4-((2-(tert-butyl)-1H-imidazol-1-yl)methyl)phenyl)-2-isobutylthiazol-5-yl)sulfonyl) carbamate using the corresponding sulfonamide (0.12 mmol, 1 equiv.) and triethylamine (0.60 mmol, 5 equiv.) except that the mixture was kept over dry ice, wherein methylchloroformate (0.14 mmol, 1.2 equiv.) was added drop-wise over approximately 2 min and the reaction mixture was stirred over dry ice for 10 min. The crude product was purified by manual FCC (0-5% with MeOH in acetonitrile) and the product was obtained as white amorphous solid (31 mg, 54% yield). ¹H NMR (400 MHz, Methanol-d₄) δ 7.98 (d, J=8.3 Hz, 2H), 7.48 (d, J=2.0 Hz, 1H), 7.35 (d, J=8.2 Hz, 2H), 7.31 (d, J=2.0 Hz, 1H), 5.51 (s, 2H), 3.46 (s, 3H), 2.89 (d, J=7.2 Hz, 2H), 2.25-2.07 (m, 2H), 1.26-1.17 (m, 2H), 1.07-1.04 (m, 8H). ¹³C NMR (101 MHz, Methanol-d₄) δ 171.4, 159.5, 152.0, 148.8, 136.3, 134.9, 134.1, 130.3, 126.9, 122.2, 118.9, 51.2, 50.3, 41.4, 29.6, 21.2, 6.8, 5.5. HRMS (ESI): calcd. for C₂₂H₂₇₄O₄S₂⁺ [M+H]⁺ 475.1468; found: 475.1457.

### EXAMPLE 27

### Butyl ((4-(4-((2-cyclopropyl-1H-imidazol-1-yl)methyl)phenyl)-2-isobutylthiazol-5-yl)sulfonyl)carbamate

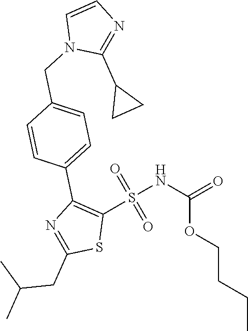 [SMILES: CCCCOC(=O)NS(=O)(=O)c1sc(CC(C)C)nc1-c1ccc(Cn2ccnc2C2CC2)cc1]

The title compound was synthesized as described for methyl((4-(4-((2-(tert-butyl)-1H-imidazol-1-yl)methyl)phenyl)-2-isobutylthiazol-5-yl)sulfonyl) carbamate using the corresponding sulfonamide (0.12 mmol, 1 equiv.) and triethylamine (0.60 mmol, 5 equiv.) except that the mixture was kept over dry ice, wherein methylchloroformate (0.15 mmol, 1.2 equiv.) was added drop-wise over approximately 2 min and the reaction mixture was stirred over dry ice for 10 min. The crude product was purified by manual FCC (0-5% with MeOH in acetonitrile) and the product was obtained as white amorphous solid (38 mg, 61% yield). ¹H NMR (400 MHz, Methanol-d₄) δ 7.97 (d, J=8.1 Hz, 2H), 7.48 (d, J=2.0 Hz, 1H), 7.36 (d, J=8.0 Hz, 2H), 7.31 (d, J=2.0 Hz, 1H), 5.52 (s, 2H), 3.87 (t, J=6.5 Hz, 2H), 2.90 (d, J=7.2 Hz, 2H), 2.27-2.08 (m, 2H), 1.48 (dq, J=8.5, 6.6 Hz, 2H), 1.37-1.26 (m, 2H), 1.26-1.19 (m, 2H), 1.06-1.05 (m, 8H), 0.89 (t, J=7.4 Hz, 3H). ¹³C NMR (126 MHz, Methanol-d₄) δ 171.6, 158.5, 152.1, 136.2, 148.9, 135.0, 134.0, 130.3, 126.9, 122.1, 119.1, 64.5, 50.3, 41.4, 30.8, 29.6, 21.2, 18.7, 12.7, 6.8, 5.5. HRMS (ESI): calcd. for C₂₅H₃₃N₄O₄S₂⁺ [M+H]⁺ 517.1938; found: 517.1950.

### EXAMPLE 28

### Methyl ((4-(4-((2-cyclopropyl-1H-imidazol-1-yl)methyl)phenyl)-2-propylthiazol-5-yl)sulfonyl)carbamate

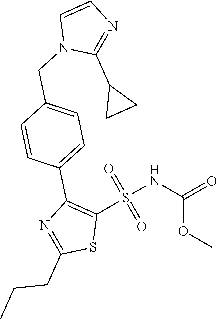 [SMILES: CCCc1nc(-c2ccc(Cn3ccnc3C3CC3)cc2)c(S(=O)(=O)NC(=O)OC)s1]

The title compound was synthesized as described for methyl((4-(4-((2-(tert-butyl)-1H-imidazol-1-yl)methyl)phenyl)-2-isobutylthiazol-5-yl)sulfonyl) carbamate using the corresponding sulfonamide sulfonamide (0.15 mmol, 1 equiv.) and triethylamine (0.73 mmol, 5 equiv.) except that the mixture was kept over dry ice, wherein methylchloroformate (0.15 mmol, 1.2 equiv.) was added drop-wise over approximately 2 min and the reaction mixture was stirred over dry ice for 10 min. The crude product was purified by manual FCC (0-5% with MeOH in acetonitrile) and the product was obtained as white amorphous solid (41 mg, 60% yield). ¹H NMR (400 MHz, Methanol-d₄) δ 7.97 (d, J=8.3 Hz, 2H), 7.50 (d, J=2.1 Hz, 1H), 7.36 (d, J=8.1 Hz, 2H), 7.34 (d, J=2.0 Hz, 1H), 5.52 (s, 2H), 3.47 (s, 3H), 3.00 (t, J=7.6 Hz, 2H), 2.26-2.17 (m, 1H), 1.86 (q, J=7.4 Hz, 2H), 1.23 (dd, J=8.4, 2.5 Hz, 2H), 1.07-1.05 (m, 5H). ¹³C NMR (101 MHz, Methanol-d₄) δ 172.6, 159.3, 152.1, 148.8, 136.0, 134.8, 134.1, 130.3, 127.0, 122.2, 118.6, 51.3, 50.4, 34.6, 23.0, 12.5, 6.9, 5.5. HRMS (ESI): calcd. for C₂₁H₂₅N₄O₄S₂⁺ [M+H]⁺ 461.1312; found: 461.1309.

### EXAMPLE 29

### Butyl ((4-(4-((2-cyclopropyl-1H-imidazol-1-yl)methyl)phenyl)-2-propylthiazol-5-yl)sulfonyl)carbamate

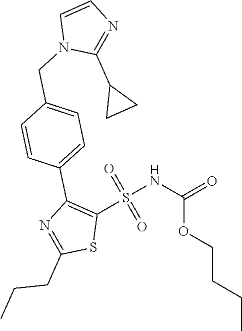 [SMILES: CCCCOC(=O)NS(=O)(=O)c1sc(CCC)nc1-c1ccc(Cn2ccnc2C2CC2)cc1]

The title compound was synthesized as described for methyl((4-(4-((2-(tert-butyl)-1H-imidazol-1-yl)methyl)phenyl)-2-isobutylthiazol-5-yl)sulfonyl) carbamate using the corresponding sulfonamide (0.15 mmol, 1 equiv.) and triethylamine (0.74 mmol, 5 equiv.) except that the mixture was kept over dry ice, wherein methylchloroformate (0.15 mmol, 1.2 equiv.) was added drop-wise over approximately 2 min and the reaction mixture was stirred over dry ice for 10 min. The crude product was purified by manual FCC (0-5% with MeOH in acetonitrile) and the product was obtained as white amorphous solid (44 mg, 59% yield). ¹1H NMR (400 MHz, Methanol-d₄) δ 7.97 (d, J=7.9 Hz, 2H), 7.49 (d, J=2.2 Hz, 1H), 7.37 (d, J=8.0 Hz, 2H), 7.32 (s, 1H), 5.51 (s, 2H), 3.89 (t, J=6.5 Hz, 2H), 2.99 (t, J=7.6 Hz, 2H), 2.26-2.13 (m, 1H), 1.85 (q, J=7.4 Hz, 2H), 1.47 (dd, J=8.5, 6.2 Hz, 2H), 1.34-1.16 (m, 4H), 1.08-1.04 (m, 5H), 0.88 (t, J=7.4 Hz, 3H). ¹³C NMR (101 MHz, Methanol-d₄) δ 172.9, 157.7, 152.5, 148.7, 135.5, 134.9, 133.9, 130.3, 127.1, 122.3, 118.4, 64.7, 50.4, 34.6, 30.7, 23.0, 18.7, 12.7, 12.6, 7.1, 5.5. HRMS (ESI): calcd. for C₂₄H₃₁N₄O₄S₂⁺ [M+H]⁺ 503.1781; found: 503.1778.

### EXAMPLE 30A

### Methyl ((4′-((2-(2-hydroxypropan-2-yl)-1H-imidazol-1-yl)methyl)-5-propyl-[1,1′-biphenyl]-2-yl)sulfonyl)carbamate

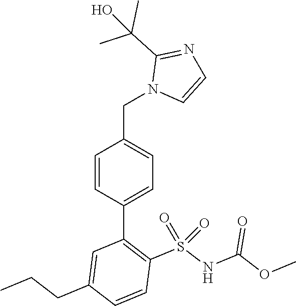 [SMILES: CCCc1ccc(S(=O)(=O)NC(=O)OC)c(-c2ccc(Cn3ccnc3C(C)(C)O)cc2)c1]

The title compound was synthesized as described for methyl ((4′-((2-(tert-butyl)-1H-imidazol-1-yl)methyl)-5-propyl-[1,1′-biphenyl]-2-yl)sulfonyl)carbamate using the corresponding butyl carbamate (17.7 mg, 34.6 μmol) and purified by FCC (2-4% MeOH in CH₂Cl₂) to afford the product as a white amorphous solid (15.6 mg, 96%). ¹H NMR (400 MHz, Methanol-d₄) δ 8.05 (d, J=8.2 Hz, 1H), 7.45-7.32 (m, 3H), 7.25 (d, J=7.9 Hz, 2H), 7.12 (d, J=1.6 Hz, 1H), 7.08 (d, J=1.8 Hz, 1H), 7.03 (d, J=1.6 Hz, 1H), 5.67 (s, 2H), 3.49 (s, 3H), 2.67 (t, J=7.7 Hz, 2H), 1.74-1.60 (m, 2H), 1.66 (s, 6H), 0.96 (t, J=7.4 Hz, 3H). ¹³C NMR (101 MHz, Methanol-d₄) δ 155.8, 153.3, 149.0, 141.9, 140.9, 137.7, 137.5, 133.5, 131.1, 130.8, 128.6, 127.9, 124.5, 123.8, 71.1, 53.1, 52.0, 38.6, 30.3, 25.3, 14.1. HRMS (ESU⁺) calcd. for C₂₇H₃₆N₃O₅S⁺ [M+H]⁺ 472.1901; found: 472.1897.

### EXAMPLE 30B

### Potassium (methoxycarbonyl) ((4′-((2-(2-hydroxypropan-2-yl)-1H-imidazol-1-yl)methyl)-5-propyl-[1,1′-biphenyl]-2-yl)sulfonyl)amide

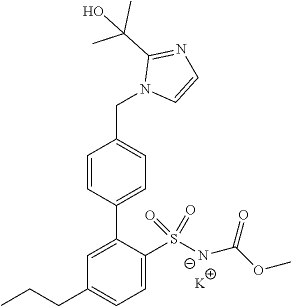 [SMILES: CCCc1ccc(S(=O)(=O)[N-]C(=O)OC)c(-c2ccc(Cn3ccnc3C(C)(C)O)cc2)c1 ; [K+]]

The title compound was synthesized as describe for potassium (butoxycarbonyl)((4′-((2-(tert-butyl)-1H-imidazol-1-yl)methyl)-5-propyl-[1,1′-biphenyl]-2-yl)sulfonyl)amide using the corresponding methyl carbamate (13.3 mg, 28 μmol) and KOH (1.70 mg, 30.2 μmol). The product was obtained as a white amorphous solid (14.3 mg, >99% yield). ¹1H NMR (400 MHz, DMSO-d₆) δ 7.86 (d, J=8.0 Hz, 1H), 7.40 (d, J=7.9 Hz, 2H), 7.21-7.14 (m, 1H), 7.10 (d, J=8.0 Hz, 2H), 6.94 (s, 1H), 6.89 (s, 1H), 6.75 (s, 1H), 5.53 (s, 2H), 3.15 (s, 3H), 2.55 (t, J=7.9 Hz, 4H),), 1.67-1.52 (m, 2H), 1.51 (s, 6H), 0.89 (t, J=7.2 Hz, 3H).

### EXAMPLE 31A

### Butyl ((4′-((2-(2-hydroxypropan-2-yl)-1H-imidazol-1-yl)methyl)-5-propyl-[1,1′-biphenyl]-2-yl)sulfonyl)carbamate

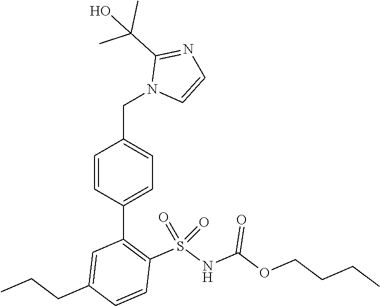 [SMILES: CCCCOC(=O)NS(=O)(=O)c1ccc(CCC)cc1-c1ccc(Cn2ccnc2C(C)(C)O)cc1]

The title compound was synthesized as described for methyl((4-(4-((2-(tert-butyl)-1H-imidazol-1-yl)methyl)phenyl)-2-isobutylthiazol-5-yl)sulfonyl) carbamate using the corresponding sulfonamide (74.3 mg, 0.156 mmol) and butyl chloroformate (27.5 μL, 0.216 mmol). The crude product was purified by FCC (5% MeOH in DCM) to afford the product as a white amorphous solid (39 mg, 47% yield). ¹1H NMR (400 MHz, Acetone-d₆) δ 8.10 (d, J=8.2 Hz, 1H), 7.45 (dd, J=8.2, 1.8 Hz, 1H), 7.34 (d, J=7.9 Hz, 2H), 7.26 (d, J=7.9 Hz, 2H), 7.16 (d, J=1.8 Hz, 1H), 6.92 (2, 1H), 6.81 (s, 1H), 5.65 (s, 2H), 3.95 (t, J=6.5 Hz, 2H), 2.71 (t, J=7.6 Hz, 2H), 1.70 (p, J=7.5 Hz, 2H), 1.63 (s, 6H), 1.51-1.38 (m, 2H), 1.30-1.14 (m, 2H), 0.95 (t, J=7.3 Hz, 3H), 0.85 (t, J=7.4 Hz, 3H). ¹³C NMR (101 MHz, Acetone-d₆) δ 153.0, 151.8, 149.1, 141.7, 139.5, 138.7, 136.6, 133.4, 131.2, 130.2, 128.4, 127.7, 126.5, 122.2, 71.1, 66.4, 50.9, 38.1, 31.3, 30.9, 24.9, 19.4, 14.0, 13.9. HRMS (ESU⁺) calcd. For C₂₇H₃₆N₃O₅S⁺ [M+H]⁺ 514.2370; found: 514.2371.

### EXAMPLE 31B

### Potassium (butoxycarbonyl) ((4′-((2-(2-hydroxypropan-2-yl)-1H-imidazol-1-yl)methyl)-5-propyl-[1,1′-biphenyl]-2-yl)sulfonyl)amide

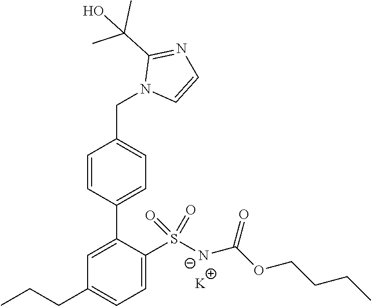 [SMILES: CCCCOC(=O)[N-]S(=O)(=O)c1ccc(CCC)cc1-c1ccc(Cn2ccnc2C(C)(C)O)cc1 ; [K+]]

The title compound was synthesized as describe for potassium (butoxycarbonyl)((4′-((2-(tert-butyl)-1H-imidazol-1-yl)methyl)-5-propyl-[1,1′-biphenyl]-2-yl)sulfonyl)amide using the corresponding butyl carbamate (15.0 mg, 29.0 μmol), and KOH (1.76 mg, 31.3 μmol) in MeOH (1.0 mL). The product was obtained as a white amorphous solid (15.7 mg, 99% yield). ¹1H NMR (400 MHz, DMSO-d₆) δ 7.86 (d, J=8.1 Hz, 1H), 7.53-7.34 (m, 2H), 7.15 (dd, J=8.1, 1.9 Hz, 1H), 7.10 (d, J=8.1 Hz, 2H), 6.93 (d, J=1.3 Hz, 1H), 6.88 (d, J=1.8 Hz, 1H), 6.74 (d, J=1.3 Hz, 1H), 5.53 (s, 2H), 5.43 (s, 1H), 3.54 (t, J=6.5 Hz, 2H), 2.60-2.49 (m, 2H), 1.68-1.54 (m, 2H), 1.52 (s, 6H), 1.45-1.26 (m, 2H), 1.26-1.14 (m, 2H), 0.89 (t, J=7.3 Hz, 3H), 0.83 (t, J=7.3 Hz, 3H).

### EXAMPLE 32

### Butyl ((4′-((2-(2-fluoropropan-2-yl)-1H-imidazol-1-yl)methyl)-5-isobutyl-[1,1′-biphenyl]-2-yl)sulfonyl)carbamate

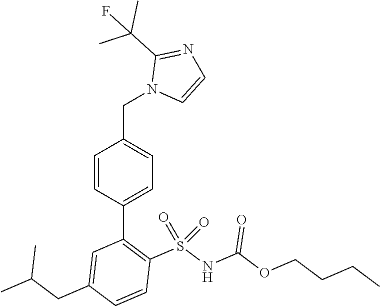 [SMILES: CCCCOC(=O)NS(=O)(=O)c1ccc(CC(C)C)cc1-c1ccc(Cn2ccnc2C(C)(C)F)cc1]

The corresponding butyl carbamate (19.0 mg, 36.1 μmol, 1 equiv.) was dissolved in CH₂Cl₂ (1 mL). The solution was cooled to −40° C. DAST (6.7 μL, 50.5 μmol, 1.4 equiv.) dropwise. The reaction mixture was stirred for 2 h while kept below −20° C. The reaction was quenched with water, extracted with CH₂Cl₂ (3×5 mL), washed with brine (5 mL), dried over Na2SO₄ and concentrated. The crude product was purified by preparative HPLC (30-70% MeCN in water with 0.05% formic acid additive) to afford the product as a white amorphous solid (6.20 mg, 36%). ¹1H NMR (400 MHz, Methanol-d₄) δ 8.06 (d, J=8.2 Hz, 1H), 7.42-7.25 (m, 3H), 7.25-7.14 (m, 2H),), 7.10-7.06 (m, 1H), 7.03 (d, J=1.6 Hz, 1H), 6.94 (d, J=1.4 Hz, 1H), 5.44 (s, 2H), 4.09-3.81 (m, 2H), 2.56 (d, J=7.2 Hz, 2H), 1.99-1.87 (m, 1H), 1.79 (d, J=21.7 Hz, 6H), 1.55-1.36 (m, 2H), 1.30-1.14 (m, 2H), 0.91 (d, J=6.6 Hz, 6H), 0.85 (t,J=7.4 Hz, 3H). ¹³C NMR (101 MHz, Methanol-d₄) δ 152.4, 149.3 (d, ²J_{C-F}=24.6 Hz), 148.5, 141.5, 139.7, 137.4, 135.7, 134.1, 130.9, 130.4, 129.2, 127.4, 127.0, 123.4, 94.5 (d, ¹J_{C-F}=165.5 Hz), 66.9, 51.3 (d, ⁴J_{C-F}=8.9 Hz), 45.6, 31.2, 30.8, 28.1 (d, ²J_{C-F}=24.5 Hz), 22.6, 19.5, 13.9. ¹⁹F NMR (376 MHz, Methanol-d₄) δ −140.02 (hept, J=21.7 Hz). HRMS (ESU⁺): calcd. for C₂₈H₃₇N₃O₄S⁺ [M+H]⁺ 530.2489; found: 530.2482.

### EXAMPLE 33A

### Methyl ((5-isobutyl-4′-((2-(thiazol-2-yl)-1H-imidazol-1-yl)methyl)-[1,1′-biphenyl]-2-yl)sulfonyl)carbamate

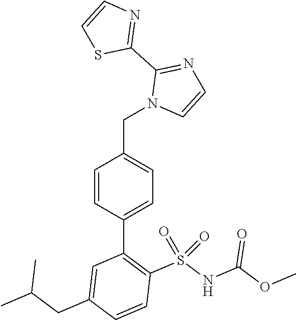 [SMILES: COC(=O)NS(=O)(=O)c1ccc(CC(C)C)cc1-c1ccc(Cn2ccnc2-c2nccs2)cc1]

The title compound was synthesized as described for methyl ((4′-((2-(tert-butyl)-1H-imidazol-1-yl)methyl)-5-propyl-[1,1′-biphenyl]-2-yl)sulfonyl)carbamate using the corresponding butyl carbamate (18.8 mg, 34.0 μmol). The crude product was concentrated and purified by FCC (4-6% MeOH in CH₂Cl₂) to afford the product as a white amorphous solid (17.3 mg, 90% yield). ¹H NMR^{a} (400 MHz, Methanol-d₄) δ 8.06 (d, J=8.2 Hz, 1H), 7.85 (d, J=3.2 Hz, 1H), 7.48 (t, J=3.3 Hz, 1H), 7.36-7.24 (m, 5H), 7.21-7.16 (m, 1H), 7.10 (d, J=1.3 Hz, 1H), 7.03 (d, J=1.8 Hz, 1H), 5.91 (s, 2H), 3.55 (s, 3H), 2.52 (d, J=7.2 Hz, 2H), 1.97-1.80 (m, 1H), 0.88 (d, J=6.6 Hz, 6H). ¹³C NMR^{a} (101 MHz, Methanol-d₄) δ 159.6, 152.7, 148.5, 144.1, 141.5, 141.0, 139.6, 137.3, 135.3, 134.0, 130.9, 130.3, 129.5, 129.1, 127.7, 124.3, 121.0, 53.4, 51.2, 45.6, 30.7, 22.6. HRMS (ESU⁺) calcd. for C₂₅H₂₇N₄O₄S₂⁺ [M+H]⁺ 511.1471; found: 511.1480.

[a] Spectrum recorded in a mixture of MeOD and CDCl₃.

### EXAMPLE 33B

### Potassium (methoxycarbonyl) ((5-isobutyl-4′-((2-(thiazol-2-yl)-1H-imidazol-1-yl)methyl)-[1,1′-biphenyl]-2-yl)sulfonyl)amide

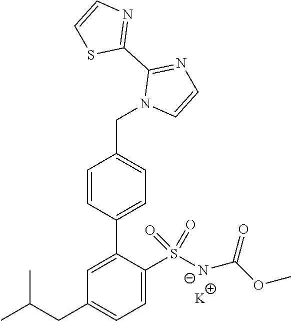 [SMILES: COC(=O)[N-]S(=O)(=O)c1ccc(CC(C)C)cc1-c1ccc(Cn2ccnc2-c2nccs2)cc1 ; [K+]]

The title compound was synthesized as described for potassium ((3-(4-((2-(tert-butyl)-1H-imidazol-1-yl)methyl)phenyl)-5-isobutylthiophen-2-yl)sulfonyl)(methoxycarbonyl)amide using_Methyl ((5-isobutyl-4′-((2-(thiazol-2-yl)-1H-imidazol-1-yl)methyl)-[1,1′-biphenyl]-2-yl)sulfonyl)carbamate (107 mg, 0.261 mmol) and butyl chloroformate (45.8 μL, 0.360 mmol). The product was obtained as a white amorphous solid (48 mg, 36%). ¹ H NMR (400 MHz, DMSO-d₆) δ 7.96 (d, J=3.3 Hz, 1H), 7.85 (d, J=8.0 Hz, 1H), 7.77 (d, J=3.3 Hz, 1H), 7.50 (d, J=1.2 Hz, 1H), 7.38 (d, J=8.0 Hz, 2H), 7.23-7.03 (m, 4H), 6.83 (s, 1H), 5.89 (s, 2H), 3.10 (s, 3H), 2.43 (d, J=7.0 Hz, 2H), 1.94-1.74 (m, 1H), 0.85 (d, J=6.6 Hz, 6H).

### EXAMPLE 34

### Methyl ((4′-((2-(2-fluoropropan-2-yl)-1H-imidazol-1-yl)methyl)-5-isobutyl-[1,1′-biphenyl]-2-yl)sulfonyl)carbamate

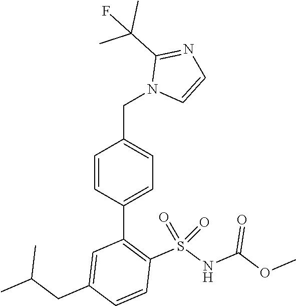 [SMILES: COC(=O)NS(=O)(=O)c1ccc(CC(C)C)cc1-c1ccc(Cn2ccnc2C(C)(C)F)cc1]

The title compound was synthesized as described for butyl ((4′-((2-(2-fluoropropan yl)-1H-imidazol-1-yl)methyl)-5-isobutyl-[1,1′-biphenyl]-2-yl)sulfonyl)carbamate using the corresponding methyl carbamate (18.2 mg, 35.6 μmol) and purified by HPLC (30-70% MeCN in water with 0.05% formic acid additive) to afford the product as a white amorphous solid (6.20 mg, 36%). ¹H NMR (400 MHz, Methanol-d₄) δ 8.06 (d, J=8.2 Hz, 1H), 7.52-7.23 (m, 6H), 7.20 (s, 1H), 7.09 (s, 1H), 5.54 (s, 2H), 3.56 (s, 3H), 2.58 (d, J=7.2 Hz, 2H), 2.01-1.90 (m, 1H1.84 (d, J=21.9 Hz, 6H), 0.92 (d, J=6.6 Hz, 6H). ¹³C NMR (101 MHz, Methanol-d₄) δ 153.3, 149.1 (d, ²J_{C-F}=19.4 Hz), 149.1, 141.9, 140.7, 137.0, 136.2, 134.4, 131.3, 130.8, 129.6, 128.1, 124.9, 124.6, 94.4 (d, ¹J_{C-F}=168.1 Hz), 53.5, 52.2 (d, ⁴J_{C-F}=8.4 Hz), 45.8, 31.2, 27.7 (d, ²J_{C-F}=24.5 Hz), 22.6. ¹⁹F NMR (376 MHz, Methanol-d₄) δ −140.67 (hept, J=21.6 Hz). HRMS (ESI⁺) calcd. for C₂₅H₃₁N₃O₄S⁺ [M+H]⁺488.2019; found: 488.2019.

### EXAMPLE 35A

### Butyl ((5-isobutyl-4′-((2-(thiazol-2-yl)-1H-imidazol-1-yl)methyl)-[1,1′-biphenyl]-2-yl)sulfonyl)carbamate

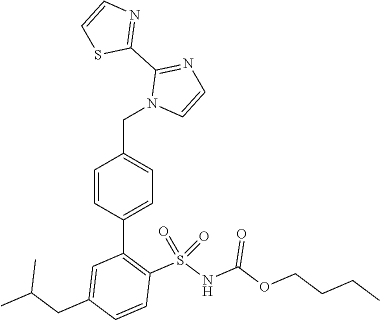 [SMILES: CCCCOC(=O)NS(=O)(=O)c1ccc(CC(C)C)cc1-c1ccc(Cn2ccnc2-c2nccs2)cc1]

The title compound was synthesized as described for butyl ((4′-((2 (tert-butyl)-1H-imidazol-1-yl)methyl)-5-propyl-[1,1′-biphenyl]-2-yl)sulfonyl)carbamate using the corresponding sulfonamide (92.7 mg, 0.205 mmol) and butyl chloroformate (36.0 μL, 0.238 μmol). The product was obtained as a white amorphous solid (40.7 mg, 36%). ¹H NMR (400 MHz, Methanol-d₄) δ 8.05 (d, J=8.2 Hz, 1H), 7.86 (d, J=3.3 Hz, 1H), 7.58 (d, J=3.3 Hz, 1H), 7.39-7.17 (m, 6H), 7.10 (d, J=1.3 Hz, 1H), 6.99 (d, J=1.8 Hz, 1H), 5.93 (s, 2H), 3.91 (t, J=6.4 Hz, 2H), 2.50 (d, J=7.2 Hz, 2H), 1.93-1.75 (m, 1H), 1.48-1.35 (m, 2H), 1.25-1.13 (m, 2H), 0.87 (d,J=6.6 Hz, 6H), 0.83 (t, J=7.4 Hz, 3H). ¹³C NMR (101 MHz, Methanol-d₄) δ 160.0, 152.7, 148.9, 144.6, 142.0, 141.4, 140.1, 138.1, 136.3, 134.4, 131.3, 130.6, 129.9, 129.4, 128.2, 125.1, 121.6, 67.0, 51.4, 45.7, 31.6, 31.1, 22.6, 19.8, 13.9. HRMS (APCI⁺) calcd. for C₂₈H₃₃N₄O₄S₂⁺ [M+H]⁺553.1938; found: 553.1935.

### EXAMPLE 35B

### Potassium (butoxycarbonyl) ((5-isobutyl-4′-((2-(thiazol-2-yl)-1H-imidazol yl)methyl)-[1,1′-biphenyl]-2-yl)sulfonyl)amide

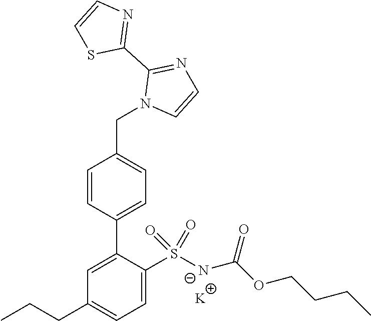 [SMILES: CCCCOC(=O)[N-]S(=O)(=O)c1ccc(CCC)cc1-c1ccc(Cn2ccnc2-c2nccs2)cc1 ; [K+]]

The title compound was synthesized as described for potassium ((3-(4-((2-(tert-butyl)-1H-imidazol-1-yl)methyl)phenyl)-5-isobutylthiophen-2-yl)sulfonyl)(methoxycarbonyl)amide using Butyl ((5-isobutyl-4′-((2-(thiazol-2-yl)-1H-imidazol-1-yl)methyl)-[1,1′-biphenyl]-2-yl)sulfonyl)carbamate (107 mg, 0.261 mmol) and butyl chloroformate (45.8 μL, 0.360 mmol). The product was obtained as a white amorphous solid (48 mg, 36%). ¹H NMR (400 MHz, DMSO-d₆) δ 7.96 (d, J=3.3 Hz, 1H), 7.84 (d, J=8.1 Hz, 1H), 7.77 (d, J=3.3 Hz, 1H), 7.49 (d, J=1.2 Hz, 1H), 7.39-7.33 (m, 2H), 7.20-7.07 (m, 4H), 6.82 (d, J=1.8 Hz, 1H), 5.89 (s, 2H), 3.51 (t, J=6.5 Hz, 2H), 2.43 (d, J=7.0 Hz, 2H), 1.91-1.72 (m, 1H), 1.39-1.25 (m, 2H), 1.25-1.09 (m, 2H), 0.85 (d, J=6.6 Hz, 6H), 0.81 (t, J=7.3 Hz, 3H).

### EXAMPLE 36

### Methyl ((5-isobutyl-4′-((2-(isopropan-2-yl)-1H-imidazol-1-yl)methyl)-[1,1′-biphenyl]-2-yl)sulfonyl)carbamate

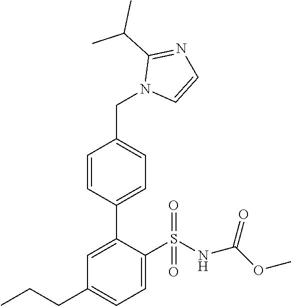 [SMILES: CCCc1ccc(S(=O)(=O)NC(=O)OC)c(-c2ccc(Cn3ccnc3C(C)C)cc2)c1]

The title compound was synthesized as described for methyl ((4′-((2-(tert-butyl)-1H-imidazol-1-yl)methyl)-5-propyl-[1,1′-biphenyl]-2-yl)sulfonyl)carbamate using the corresponding butyl carbamate (48.0 mg, 93.8 μmol) in MeOH (1 mL). The crude product was concentrated and purified by FCC (5-7% MeOH in CH₂Cl₂) to afford the product as a white amorphous solid (41.9 mg, 95% yield). ¹H NMR (400 MHz, Methanol-d₄) δ 8.24-7.83 (m, 1H), 7.39-7.28 (m, 2H), 7.27-7.16 (m, 2H), 7.17-7.05 (m, 3H), 6.90 (d, J=1.9 Hz, 1H), 5.27 (s, 2H), 3.32-3.12 (m, 1H), 2.43 (d, J=7.2 Hz, 2H), 1.88-1.65 (m, 1H), 1.21 (d, J=7.0 Hz, 6H), 0.81 (d, J=6.6 Hz, 6H). ¹³C NMR (101 MHz, Methanol-d₄) δ 158.6, 153.8, 146.9, 142.1, 141.3, 139.3, 135.5, 133.9, 131.2, 130.6, 129.1, 127.7, 122.7, 122.5, 52.8, 51.1, 45.8, 31.2, 26.7, 22.7, 21.3. HRMS (ESU⁺) calcd. for C₂₅H₃₂N₃O₄S⁺ [M+H]⁺ 470.2114; found: 470.2107.

### EXAMPLE 37A

### Butyl ((5-isobutyl-4′-((2-(isopropan-2-yl)-1H-imidazol-1-yl)methyl)-[1,1′-biphenyl]-2-yl)sulfonyl)carbamate

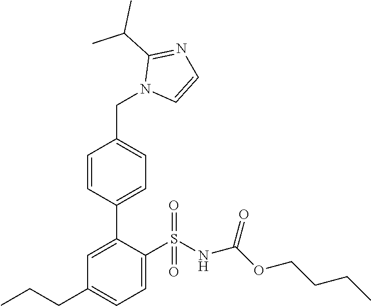 [SMILES: CCCCOC(=O)NS(=O)(=O)c1ccc(CCC)cc1-c1ccc(Cn2ccnc2C(C)C)cc1]

The title compound was synthesized as described for butyl ((4′-((2 (tert-butyl)-1H-imidazol-1-yl)methyl)-5-propyl-[1,1′-biphenyl]-2-yl)sulfonyl)carbamate using the corresponding sulfonamide (97.6 mg, 0.237 mmol) and butyl chloroformate (41.8 μL, 0.327 mmol). The crude product was purified by FCC (3-5% MeOH in CH₂Cl₂) to afford the product as a white amorphous solid (72 mg, 59% yield). ¹H NMR (400 MHz, Acetone-d₆) δ 8.13 (d, J=8.2 Hz, 1H), 7.60-7.35 (m, 3H), 7.29-7.13 (m, 2H), 7.10 (d, J=1.8 Hz, 1H), 7.00 (d, J=1.4 Hz, 1H), 6.83 (d, J=1.4 Hz, 1H), 6.47 (bs, 1H), 5.30 (s, 2H), 3.94 (t, J=6.5 Hz, 2H), 3.44-3.08 (m, 1H), 2.60 (d, J=7.2 Hz, 2H), 2.01-1.81 (m, 1H), 1.71-1.38 (m, 2H), 1.25 (d, J=6.9 Hz, 6H), 1.23-1.16 (m, 2H), 0.92 (d, J=6.6 Hz, 6H), 0.85 (t, J=7.4 Hz, 3H). ¹³C NMR (101 MHz, Acetone-d₆) δ 154.4, 153.0, 146.8, 141.1, 141.0, 138.5, 136.5, 133.7, 131.0, 130.6, 128.7, 127.4, 125.1, 120.7, 65.6, 50.0, 45.3, 31.5, 30.7, 26.4, 22.6, 21.8, 19.5, 14.0. HRMS (ESI⁺) calcd. for C₂₈H₃₈N₃O₄S⁺ [M+H]⁺ 512.2583; found: 512.2579.

### EXAMPLE 37B

### Potassium (butoxycarbonyl) ((5-isobutyl-4′-((2-(isopropan-2-yl)-1H-imidazol-1-yl)methyl)-[1,1′-biphenyl]-2-yl)sulfonyl)amide

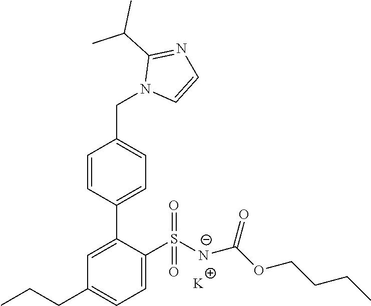 [SMILES: CCCCOC(=O)[N-]S(=O)(=O)c1ccc(CCC)cc1-c1ccc(Cn2ccnc2C(C)C)cc1 ; [K+]]

The title compound was synthesized as described for potassium (butoxycarbonyl)((4′-((2-(tert-butyl)-1H-imidazol-1-yl)methyl)-5-propyl-[1,1′-biphenyl]-2-yl)sulfonyl)amide using the corresponding butyl carbamate (13.7 mg, 26.6 μmol) and KOH (1.61 mg, 28,7 μmol) in MeOH (0.59 M). The product was obtained as a white amorphous solid (8.5 mg, 58% yield), ¹H NMR (400 MHz, Acetone-d₆) δ 8.06 (d, J=8.4 Hz, 1H), 7.52 (d, J=7.8 Hz, 2H), 7.25-7.13 (m, 1H), 7.12-7.04 (m, 2H), 6.99 (d, J=1.2 Hz, 1H), 6.96 (s, 1H), 6.84 (d, J=1.3 Hz, 1H), 5.24 (s, 2H), 3.68 (t, J=6.6 Hz, 2H), 3.20-3.01 (m, 1H), 2.52 (d, J=7.1 Hz, 2H), 1.95-1.79 (m, 1H), 1.51-1.34 (m, 2H), 1.33-1.23 (m, 2H), 1.22 (d, J=6.8 Hz, 6H), 0.91 (d, J=6.6 Hz, 6H), 0.86 (t, J=7.3 Hz, 3H).

### EXAMPLE 38A

### Butyl ((5-isobutyl-4′-((2-(cyclopropan-2-yl)-1H-imidazol-1-yl)methyl)-[1,1′-biphenyl]-2-yl)sulfonyl)carbamate

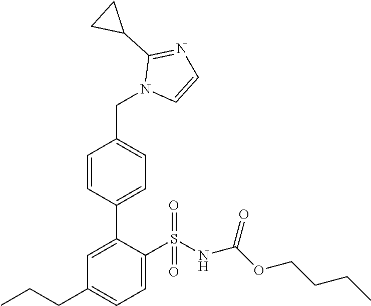 [SMILES: CCCCOC(=O)NS(=O)(=O)c1ccc(CCC)cc1-c1ccc(Cn2ccnc2C2CC2)cc1]

The title compound was synthesized as described for butyl ((4′-((2 (tert-butyl)-1H-imidazol-1-yl)methyl)-5-propyl-[1,1′-biphenyl]-2-yl)sulfonyl)carbamate using the corresponding sulfonamide (77.4 mg, 0.189 mmol) and butyl chloroformate (33.3 μL, 0.261mmo1). The crude product was purified by FCC (3-5% MeOH in CH₂Cl₂) to afford the product as a white amorphous solid (41 mg, 38% yield). ¹1H NMR^{a} (400 MHz, Methanol-d₄) δ 8.05 (d, J=8.2 Hz, 1H), 7.44-7.32 (m, 2H), 7.29 (dd, J=8.2, 1.8 Hz, 1H), 7.19 (d, J=8.1 Hz, 2H), 7.08 (d, J=1.6 Hz, 1H), 7.03 (d, J=1.8 Hz, 1H), 6.92 (d, J=1.6 Hz, 1H), 5.33 (s, 2H), 3.91 (t, J=6.5 Hz, 2H), 2.54 (d, J=7.2 Hz, 2H), 2.15-1.80 (m, 2H), 1.58-1.37 (m, 2H), 1.32-1.12 (m, 2H), 1.06-0.93 (m, 4H), 0.90 (d, J=6.6 Hz, 6H), 0.84 (t, J=7.4 Hz, 3H). ¹³C NMR^{a} (101 MHz, Methanol-d₄) δ 154.8, 150.0, 147.4, 141.1, 140.6, 137.1, 135.8, 133.7, 130.6, 130.5, 128.9, 127.2, 124.2, 121.6, 66.3, 50.4, 45.6, 31.3, 30.7, 22.6, 19.5, 13.9, 7.6, 7.2. HRMS (ESU⁺) calcd. for C₂₈H₃₆N₃O₄S⁺ [M+H]⁺ 512.2583 found 512.2579.

[a] Recorded in a mixture of MeOD and CDCl₃.

### Example 38B

### Potassium (butoxycarbonyl) ((5-isobutyl-4′-((2-(cyclopropan-2-yl)-1H-imidazol-1-yl)methyl)-[1,1′-biphenyl]-2-yl)sulfonyl)amide

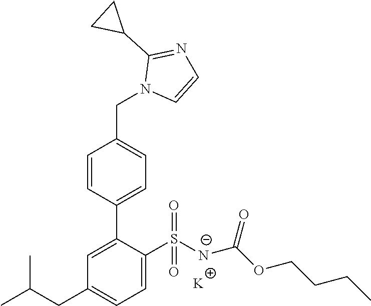 [SMILES: CCCCOC(=O)[N-]S(=O)(=O)c1ccc(CC(C)C)cc1-c1ccc(Cn2ccnc2C2CC2)cc1 ; [K+]]

The title compound was synthesized as described for potassium (butoxycarbonyl)((4′-((2-(tert-butyl)-1H-imidazol-1-yl)methyl)-5-propyl-[1,1′-biphenyl]-2-yl)sulfonyl)amide using the corresponding butyl carbamate (13.5 mg, 26.2 μmol) and KOH (1.59 mg, 28.3 μmol) in MeOH (0.59 M). The product was obtained as a white amorphous solid (4.4 mg, 31% yield), 1H NMR (400 MHz, Acetone-d₆) δ 8.06 (d, J=7.2 Hz, 1H), 7.54 (d, J=8.2 Hz, 2H), 7.14 (t, J=7.8 Hz, 3H), 7.02 (d, J=1.3 Hz, 1H), 6.94 (d, J=1.9 Hz, 1H), 6.75 (d, J=1.3 Hz, 1H), 5.31 (s, 2H), 3.64 (t, J=6.7 Hz, 2H), 2.51 (d, J=7.1 Hz, 2H), 1.99-1.77 (m, 4H), 1.46-1.30 (m, 2H), 1.31-1.19 (m, 2H), 0.91 (d, J=6.6 Hz, 6H), 0.89-0.86 (m, 3H), 0.86-0.83 (m, 3H).

### EXAMPLE 39A

### Methyl ((5-isobutyl-4′-((2-(cyclopropan-2-yl)-1H-imidazol-1-yl)methyl)-[1,1′-biphenyl]-2-yl)sulfonyl)carbamate

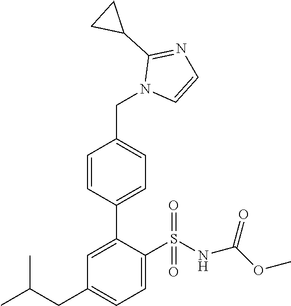 [SMILES: COC(=O)NS(=O)(=O)c1ccc(CC(C)C)cc1-c1ccc(Cn2ccnc2C2CC2)cc1]

The title compound was synthesized as described for methyl ((4′-((2-(tert-butyl)-1H-imidazol-1-yl)methyl)-5-propyl-[1,1′-biphenyl]-2-yl)sulfonyl)carbamate using the corresponding butyl carbamate (27.4 mg, 53.8 μmol) in MeOH (1 mL). The crude product was concentrated and purified by FCC (5-7% MeOH in CH₂Cl₂) to afford the product as a white amorphous solid (21.6 mg, 86% yield). ¹H NMR (400 MHz, Methanol-d₄) δ 8.05 (d, J=8.2 Hz, 1H), 7.42-7.36 (m, 2H), 7.32-7.21 (m, 4H), 7.08 (d, J=1.8 Hz, 1H), 7.01 (d, J=1.8 Hz, 1H), 5.41 (s, 2H), 3.43 (s, 3H), 2.64-2.35 (m, 3H), 2.25-2.02 (m, 1H), 1.97-1.81 (m, J=6.7 Hz, 1H), 1.19-1.03 (m, 2H), 1.03-0.97 (m, 2H), 0.91 (d, J=6.6 Hz, 6H). ¹³C NMR (101 MHz, Methanol-d₄) δ 158.0, 150.4, 147.2, 141.9, 141.4, 138.9, 135.8, 133.9, 131.1, 130.7, 129.2, 127.9, 122.7, 122.6, 52.9, 51.1, 45.8, 31.2, 22.7, 7.9, 7.3. HRMS (ESU⁺) calcd. for C₂₅H₃₀N₃O₄S⁺ [M+H]⁺ 468.1957; found: 468.1947.

### EXAMPLE 39B

### Potassium (methoxycarbonyl) ((5-isobutyl-4′-((2-(cyclopropan-2-yl)-1H-imidazol-1-yl)methyl)-[1,1′-biphenyl]-2-yl)sulfonyl)amide

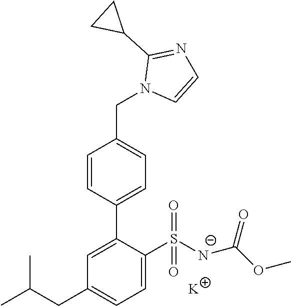 [SMILES: COC(=O)[N-]S(=O)(=O)c1ccc(CC(C)C)cc1-c1ccc(Cn2ccnc2C2CC2)cc1 ; [K+]]

The title compound was synthesized as described for potassium (butoxycarbonyl)((4′-((2-(tert-butyl)-1H-imidazol-1-yl)methyl)-5-propyl-[1,1′-biphenyl]-2-yl)sulfonyl)amide using the corresponding methyl carbamate (21.6 mg, 45.7 μmol) and KOH (2.77 mg, 49.3 μmol) in MeOH (0.59 M). The product was obtained as a white amorphous solid (20.3 mg, 88% yield). ¹H NMR (400 MHz, Acetone-d₆) δ 8.06 (d, J=8.1 Hz, 1H), 7.61-7.48 (m, 2H), 7.22-7.08 (m, 3H), 7.03 (d, J=1.3 Hz, 1H), 6.95 (d, J=1.8 Hz, 1H), 6.75 (d, J=1.3 Hz, 1H), 5.31 (s, 2H), 3.23 (s, 3H), 2.52 (d, J=7.2 Hz, 2H), 1.99-1.83 (m, 2H), 0.91 (d, J=6.6 Hz, 6H), 0.89-0.81 (m, 4H).

### EXAMPLE 40

### Butyl ((5-isobutyl-3-(4-(2-(2-hydroxypropan-2-yl)-1H-imidazol-1-yl)methyl)phenyl)-2-yl)sulfonyl)carbamate

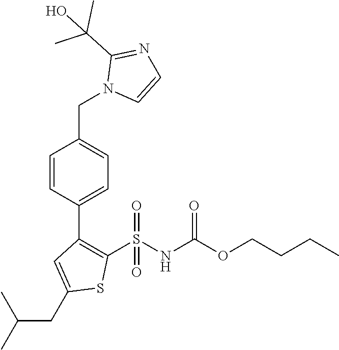 [SMILES: CCCCOC(=O)NS(=O)(=O)c1sc(CC(C)C)cc1-c1ccc(Cn2ccnc2C(C)(C)O)cc1]

The title compound was synthesized as described for butyl ((4′-((2 (tert-butyl)-1H-imidazol-1-yl)methyl)-5-propyl-[1,1:-biphenyl]-2-yl)sulfonyl)carbamate using the corresponding sulfonamide (19.9 mg, 46.0 μmol) and butyl chloroformate (8.7 μL, 63.5 μmol). The crude product was purified by HPLC (30-50% water in MeCN with 0.05% formic acid) to afford the product as a white amorphous solid (10.3 mg, 42% yield) after lyophilisation. ¹H NMR (400 MHz, Methanol-d₄) δ 7.67-7.54 (m, 2H), 7.26 (d, J=8.1 Hz, 2H), 7.11 (d, J=1.6 Hz, 1H), 7.05 (d, J=1.6 Hz, 1H), 6.83 (s, 1H), 5.66 (s, 2H), 3.91 (t, J=6.5 Hz, 2H), 2.73 (d, J=7.1 Hz, 2H), 2.01-1.86 (m, 1H), 1.65 (s, 6H), 1.57-1.40 (m, 2H), 1.37-1.18 (m, 2H), 1.00 (d, J=6.6 Hz, 6H), 0.88 (t, J=7.4 Hz, 3H). ¹³C NMR (101 MHz, Methanol-d₄) δ 156.5, 153.1, 150.0, 144.9, 137.7, 136.0, 132.2, 130.8, 130.3, 128.3, 123.9, 123.7, 71.0, 66.5, 52.1, 40.0, 31.9, 31.8, 30.2, 22.6, 20.0, 14.1. HRMS (ESU⁺) calcd. for C₂₆H₃₆N₃O₅S₂⁺ [M+H]⁺ 534.2096; found: 534.2097.

### EXAMPLE 41

### Methyl ((5-isobutyl-3-(4-(2-(2-hydroxypropan-2-yl)-1H-imidazol-1-yl)methyl)phenyl)-2-yl)sulfonyl)carbamate

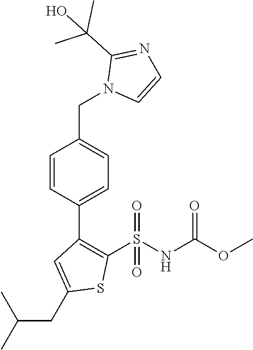 [SMILES: COC(=O)NS(=O)(=O)c1sc(CC(C)C)cc1-c1ccc(Cn2ccnc2C(C)(C)O)cc1]

The title compound was synthesized as described for methyl ((4′-((2-(tert-butyl)-1H-imidazol-1-yl)methyl)-5-propyl-[1,1′-biphenyl]-2-yl)sulfonyl)carbamate using the corresponding butyl carbamate (10.0 mg, 18.7 μmol) in MeOH (1 mL). The crude product was concentrated and purified by FCC (3-6% MeOH in CH₂Cl₂) to afford the product as a white amorphous solid (6.0 mg, 65% yield). ¹H NMR (400 MHz, Methanol-d₄) δ 7.58 (d, J=8.3 Hz, 2H), 7.26 (d, J=8.2 Hz, 2H), 7.14 (d, J=1.7 Hz, 1H), 7.09 (d, J=1.6 Hz, 1H), 6.81 (s, 1H), 5.66 (s, 2H), 3.48 (s, 3H), 2.72 (d, J=7.1 Hz, 2H), 2.10-1.85 (m, 1H), 1.65 (s, 6H), 1.00 (d, J=6.6 Hz, 6H). HRMS (ESU⁺) calcd. for C₂₃H₃₀N₃O₅S₂⁺ [M+H]⁺ 492.1627; found: 492.1628.

### EXAMPLE 42

Butyl ((5-isobutyl-3-(4-(2-(2-fluoropropan-2-yl)-1H-imidazol-1-yl)methyl)phenyl)-2-yl)sulfonyl)carbamate

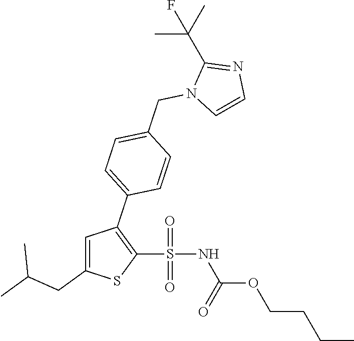 [SMILES: CCCCOC(=O)NS(=O)(=O)c1sc(CC(C)C)cc1-c1ccc(Cn2ccnc2C(C)(C)F)cc1]

The title compound was synthesized as described for butyl ((4′-((2-(2-fluoropropan yl)-1H-imidazol-1-yl)methyl)-5-isobutyl-[1,1′-biphenyl]-2-yl)sulfonyl)carbamate using the corresponding butyl carbamate (21.6 mg, 38.4 μmol) and DAST (5.1 μL, 38.4 μmol) and purified by HPLC (30-70% water in MeCN with 0.05% formic acid) to afford the product as a white amorphous solid (5.7 mg, 28% yield) after lyophilisation. ¹H NMR (400 MHz, Methanol-d₄) δ 7.59-7.46 (m, 2H), 7.24 (d, J=8.0 Hz, 2H), 7.10 (s, 1H), 6.97 (s, 1H), 6.87 (s, 1H), 5.46 (s, 2H), 3.98 (t, J=6.4 Hz, 2H), 2.75 (d, J=7.1 Hz, 2H), 2.02-1.87 (m, 1H), 1.77 (d, J=21.6 Hz, 6H), 1.58-1.40 (m, 2H), 1.34-1.15 (m, 2H), 1.00 (d, J=6.6 Hz, 6H), 0.88 (t, J=7.4 Hz, 3H). ¹³C NMR (101 MHz, Methanol-d₄) δ 151.5, 150.6, 148.4 (d, ²J_{C-F}=25.5 Hz), 145.2, 137.4, 133.9, 131.9, 129.4, 129.2, 126.8, 125.7, 122.6, 93.5 (d, ¹J_{C-F}=165.5 Hz), 65.7, 50.2 (d, ⁴JC-F=8.9 Hz), 38.6, 30.4, 30.3, 26.7 (d, ²J_{C-F}=24.6 Hz), 21.1, 18.5, 12.6. ¹⁹F NMR (376 MHz, Methanol-d₄) δ -140.66 (hept, J=21.5 Hz). HRMS (ESU⁺) calcd. for C₂₆H₃₆FN₃O₄S₂⁺ [M+H]⁺ 536.2053; found: 536.2051.

### EXAMPLE 43

### Methyl ((5-isobutyl-3-(4-(2-(2-fluoropropan-2-yl)-1H-imidazol-1-yl)methyl)phenyl)-2-yl)sulfonyl)carbamate

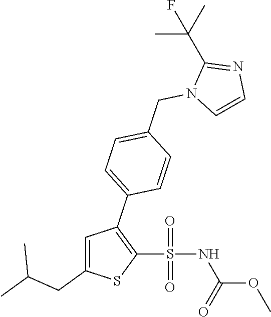 [SMILES: COC(=O)NS(=O)(=O)c1sc(CC(C)C)cc1-c1ccc(Cn2ccnc2C(C)(C)F)cc1]

The title compound was synthesized as described for butyl ((4′-((2-(2-fluoropropan-2-yl)-1H-imidazol-1-yl)methyl)-5-isobutyl-[1,1′-biphenyl]-2-yl)sulfonyl)carbamate using the corresponding methyl carbamate (18.9 mg, 38.5 μmol) and purified by HPLC (30-70% water in MeCN with 0.05% formic acid) to afford the product as a white amorphous solid (6.5 mg, 34% yield) after lyophilisation. ¹H NMR (400 MHz, Methanol-d₄) δ 7.53-7.35 (m, 2H), 7.21-7.10 (m, 3H), 7.05 (d, J=1.6 Hz, 1H), 6.77 (s, 1H), 5.41 (s, 2H), 3.48 (s, 3H), 2.65 (d, J=7.1 Hz, 2H), 1.96-1.78 (m, 1H), 1.70 (d, J=21.9 Hz, 6H), 0.90 (d, J=6.6 Hz, 6H). ¹³C NMR (101 MHz, Methanol-d₄) δ 153.3, 152.3, 149.4 (d, ²J_{C-F}=24.8 Hz), 146.9, 138.1, 135.5, 133.0, 130.8, 128.3, 125.6, 124.5, 121.4, 94.8 (d, ¹J_{C-F}=168.1Hz), 53.6, 52.0 (d, ⁴J_{C-F}=9.0 Hz), 40.0, 31.8, 27.8 (d, ²J_{C-F}=24.4 Hz), 22.5. ¹⁹F NMR (376 MHz, Methanol-d₄) δ −140.93 (hept, J=21.8 Hz). HRMS (ESU⁺) calcd. for C₂₃H₂₉FN₃O₄S₂⁺ [M+H]⁺ 494.1584; found: 494.1588.

### EXAMPLE 44

### 2-methoxyethyl ((5-isobutyl-3-(4-(2-(2-tertbutyl)-1H-imidazol-1-yl)methyl)phenyl)-2-yl)sulfonyl)carbamate

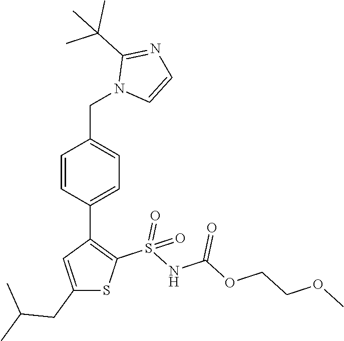 [SMILES: COCCOC(=O)NS(=O)(=O)c1sc(CC(C)C)cc1-c1ccc(Cn2ccnc2C(C)(C)C)cc1]

The title compound was synthesized as described for butyl ((4-(4-((2-(2-hydroxypropan-2-yl)-1H-imidazol-1-yl)methyl)phenyl)-2-propyl thiazol-5-yl)sulfonyl)carbamate using the corresponding sulfonamide (50.0 mg, 0.116 mmol) and 2-methoxyethyl chloroformate (16.2 μL, 0.139 mmol). The crude product was purified by FCC (0-5% with MeOH in MeCN) to afford the product was as a white amorphous solid (36 mg, 58% yield). ¹H NMR (400 MHz, Acetone-d₆) δ 7.65 (d, J=7.9 Hz, 2H), 7.16 (d, J=7.8 Hz, 2H), 7.01 (bs, 1H), 6.96 (s, 1H), 6.92 (bs, 1H), 5.55 (s, 2H), 4.11 (t, J=4 Hz, 2H), 3.46 (t, J=4 Hz, 2H), 3.26 (s, 3H), 2.79 (d, J=7.0 Hz, 2H), 1.98 (dq, J=13.4, 6.7 Hz, 1H), 1.44 (s, 9H), 1.01 (d, J=6.5 Hz, 6H). ¹³C NMR (101 MHz, Acetone-d₆) δ 153.6, 151.9, 149.7, 144.4, 137.7, 134.0, 133.3, 129.6, 129.4, 126.3, 124.7, 122.3, 70.0, 64.7, 57.8, 50.6, 38.6, 33.3, 30.4, 29.2, 21.6. HRMS (ESU⁺): calcd. for C₂₆H₃₆N₃O₅S₂⁺ [M+H]⁺ 534.2091; found: 534.2092.

### EXAMPLE 45

### Butyl ((4-(4-((2-(2-fluoropropan-2-yl)-1H-imidazol-1-yl)methyl)phenyl)-2-propyl thiazol-5-yl)sulfonyl)carbamate

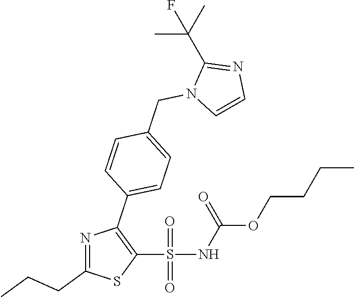 [SMILES: CCCCOC(=O)NS(=O)(=O)c1sc(CCC)nc1-c1ccc(Cn2ccnc2C(C)(C)F)cc1]

The title compound was synthesized as described for butyl ((4′-((2-(2-fluoropropan-2-yl)-1H-imidazol-1-yl)methyl)-5-isobutyl-[1,1′-biphenyl]-2-yl)sulfonyl)carbamate using the corresponding butyl carbamate (40.0 mg, 0.073 mmol) and DAST (13.5 μL, 0.102 mmol). The crude product was purified by preparative HPLC (15-80% with water in acetonitrile) and the product was obtained as a white amorphous solid (19.0 mg, 45% yield).) after lyophilisation. ¹H NMR (400 MHz, Acetone-d₆) δ 7.91 (d, J=7.9 Hz, 2H), 7.28 (d, J=7.9 Hz, 2H), 7.14 (bs, 1H), 6.98 (bs, 1H), 5.53 (s, 2H), 4.04 (t, J=6.5 Hz, 2H), 3.07 (t, J=7.5 Hz, 2H), 1.96-1.85 (m, 2H), 1.77 (d, J=21.6 Hz, 6H), 1.50 (p, J=6.8 Hz, 2H), 1.26 (h, J=7.4 Hz, 2H), 1.07 (t, J=7.3 Hz, 3H), 0.88 (t, J=7.4 Hz, 3H). ¹³C NMR (101 MHz, Acetone-d₆) δ 174.7, 155.7, 150.6, 148.0 (d,²J_{C-F}=25.0 Hz), 139.3, 132.4, 130.4, 130.1, 126.7, 126.5, 122.6, 93.9 (d, ¹J_{C-F}=164.0 Hz). 66.1, 50.1 (d, ⁴J_{C-F}=8.3 Hz), 35.0, 30.4, 27.2 (d, ²J_{C-F}=24.6 Hz), 22.7, 18.5, 12.9, 12.9. ¹⁹F NMR (376 MHz, Acetone-d₆) δ −139.3 (hept, J=21.5 Hz). HRMS (ESU⁺): calcd. for C₂₄H₃₂FN₄O₄S_{2hu +} [M+H]⁺ 523.1844; found: 523.1835.

### EXAMPLE 46

### Methyl ((4-(4-((2-(2-fluoropropan-2-yl)-1H-imidazol-1-yl)methyl)phenyl)-2-propyl thiazol-5-yl)sulfonyl)carbamate

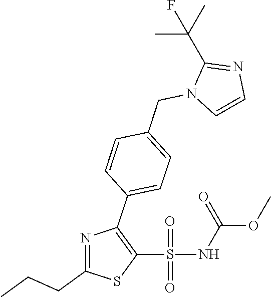 [SMILES: CCCc1nc(-c2ccc(Cn3ccnc3C(C)(C)F)cc2)c(S(=O)(=O)NC(=O)OC)s1]

The title compound was synthesized as described for butyl ((4′-((2-(2-fluoropropan-2-yl)-1H-imidazol-1-yl)methyl)-5-isobutyl-[1,1′-biphenyl]-2-yl)sulfonyl)carbamate using the corresponding methyl carbamate (40 mg, 0.0794 mmol) and DAST (0.015 mL, 0.111 mmol). The crude product was purified by preparative HPLC, (15-80% with water in acetonitrile) and the product was obtained as a white amorphous solid (17 mg, 40% yield) after lyophilisation. ¹H NMR (400 MHz, Acetone-d₆) δ 7.75 (d, J=8.2 Hz, 2H), 7.12 (d, J=8.0 Hz, 2H), 7.01 (bs, 1H), 6.83 (bs, 1H), 5.38 (s, 2H), 3.49 (s, 3H), 2.93 (t, J=7.6 Hz, 2H), 1.80-1.70 (m, 2H), 1.62 (d, J=21.6 Hz, 6H), 0.93 (t, J=7.4 Hz, 3H). ¹³C NMR (101 MHz, Acetone-d₆) δ 174.8, 155.8, 151.3, 148.0 (d,²J_{C-F}=25.2 Hz), 139.3, 132.4, 130.2, 130.1, 126.6, 126.6, 122.6, 94.0 (d, ¹J_{C-F}=163.9 Hz), 52.7, 50.1 (d, ⁴J_{C-F}=8.3 Hz), 34.9, 27.2 (d, ²J_{C-F}=24.6 Hz), 22.7, 12.9. ¹⁹F NMR (376 MHz, Acetone-d₆) δ −139.5 (hept, J=21.4 Hz). HRMS (ESU⁺): calcd. For C₂₁H₂₆FN₄O₄S₂ [M+H]⁺ 481.1374; found: 481.1378.

### EXAMPLE 47

### Butyl ((4-(4-((2-(2-fluoropropan-2-yl)-1H-imidazol-1-yl)methyl)phenyl)-2-isobutylthiazol-5-yl)sulfonyl)carbamate

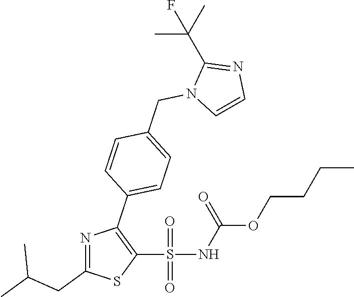 [SMILES: CCCCOC(=O)NS(=O)(=O)c1sc(CC(C)C)nc1-c1ccc(Cn2ccnc2C(C)(C)F)cc1]

The title compound was synthesized as described for butyl ((4′-((2-(2-fluoropropan-2-yl)-1H-imidazol-1-yl)methyl)-5-isobutyl-[1,1′-biphenyl]-2-yl)sulfonyl)carbamate using the corresponding butyl carbamate (40 mg, 0.071 mmol) and DAST (13.0 μL, 0.100 mmol). The crude product was purified by preparative HPLC (15-80% with water in MeCN) and the product was obtained as a white amorphous solid (20 mg, 47% yield) after lyophilisation. ¹H NMR (400 MHz, Acetone-d₆) δ 7.91 (d, J=8.3 Hz, 2H), 7.27 (d, J=8.2 Hz, 2H), 7.11 (bs, 1H), 6.94 (bs, 1H), 5.51 (s, 2H), 4.04 (t, J=6.5 Hz, 2H), 2.98 (d, J=7.1 Hz, 2H), 2.20 (dt, J=13.5, 6.8 Hz, 1H), 1.76 (d, J=21.5 Hz, 6H), 1.50 (dq, J=8.4, 6.6 Hz, 2H), 1.35-1.19 (m, 2H), 1.05 (d, J=6.7 Hz, 6H), 0.88 (t, J=7.4 Hz, 3H). ¹³C NMR (101 MHz, Acetone-d₆) δ 173.7, 155.7, 150.7, 148.1 (d, ²J_{C-F}=24.8 Hz), 139.4, 132.4, 130.5, 130.1, 126.8, 126.7, 122.5, 94.0 (d, ¹J_{C-F}=163.5 Hz). 66.1, 50.0 (d, ⁴J_{C-F}=8.3 Hz), 41.7, 30.4, 29.4, 27.3 (d, ²J_{C-F}=24.6 Hz), 21.6, 18.6, 13.0. ¹⁹ F NMR (376 MHz, Acetone-d₆) δ −139.3 (hept, J=21.6 Hz). HRMS (ESU⁺): calcd. For C₂₅H₃₄FN₄O₄S₂⁺ [M+H]⁺ 537.2000; found: 537.1992.

### EXAMPLE 48

### Methyl ((4-(4-((2-(2-fluoropropan-2-yl)-1H-imidazol-1-yl)methyl)phenyl)-2-isobutylthiazol-5-yl)sulfonyl)carbamate

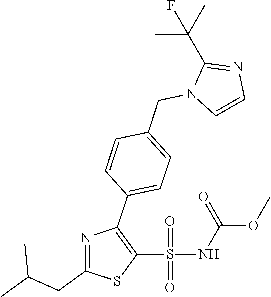 [SMILES: COC(=O)NS(=O)(=O)c1sc(CC(C)C)nc1-c1ccc(Cn2ccnc2C(C)(C)F)cc1]

The title compound was synthesized as described for butyl ((4′-((2-(2-fluoropropan-2-yl)-1H-imidazol-1-yl)methyl)-5-isobutyl-[1,1′-biphenyl]-2-yl)sulfonyl)carbamate using the corresponding butyl carbamate (40.0 mg, 0.077 mmol) and DAST (14.0 μL, 0.108 mmol). The crude product was purified by preparative HPLC (15-80% with water in acetonitrile) and the product was obtained as a white amorphous solid (21 mg, 50% yield) after lyophilisation. ¹H NMR (400 MHz, Acetone-d₆) δ 7.90 (d, J=8.2 Hz, 2H), 7.27 (d, J=8.0 Hz, 2H), 7.17 (bs, 1H), 7.00 (bs, 1H), 5.54 (s, 2H), 3.63 (s, 3H), 2.98 (d, J=7.1 Hz, 2H), 2.25-2.15 (m, 1H), 1.78 (d, J=21.7 Hz, 6H), 1.05 (d, J=6.6 Hz, 6H). ¹³C NMR (101 MHz, Acetone-d₆) δ 173.9, 161.5, 155.9, 151.1, 147.9 (d, ²J_{C-F}=25.4 Hz), 139.2, 132.4, 130.1, 126.7, 126.3, 122.7, 93.9 (d, ¹J_{C-F}=164.4 Hz). 52.76, 50.2 (d, ⁴J_{C-F}=8.4 Hz), 41.7, 29.4, 27.2 (d, ²J_{C-F}=24.4 Hz), 21.5. ¹⁹F NMR (376 MHz, Acetone-d₆) δ −139.6 (hept, J=21.6 Hz). HRMS (ESU⁺): calcd. For C₂₂H₂₈FN₄O₄S₂⁺ [M+H]⁺ 495.1531; found: 495.1532.

### EXAMPLE 49

### Butyl ((4′-((2-(tert-butyl)-1H-imidazol-1-yl)methyl)-3′-fluoro-5-isobutyl-[1,1′-biphenyl]-2-yl)sulfonyl)carbamate

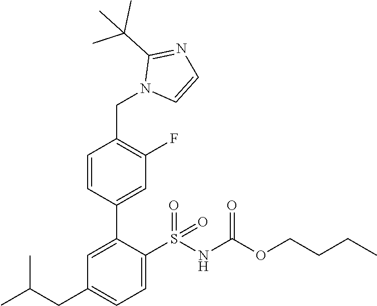 [SMILES: CCCCOC(=O)NS(=O)(=O)c1ccc(CC(C)C)cc1-c1ccc(Cn2ccnc2C(C)(C)C)c(F)c1]

The title compound was synthesized as described for butyl ((4′-((2 (tert-butyl)-1H-imidazol-1-yl)methyl)-5-propyl-[1,1′-biphenyl]-2-yl)sulfonyl)carbamate with the exception of the butyl chloroformate (42.9 μL, 0.336 mmol, 1.2 equiv.) being added to the corresponding sulfonamide (125 mg, 0.280 mmol, 1 equiv.) at 0° C. and the reaction was quenched after 1 h. The crude product was purified by FCC (5% MeOH in CH₂Cl₂) to afford the product as a white amorphous solid (58.8 mg, 39% yield). ¹1H NMR (400 MHz, Methanol-d₄) δ 8.05 (d, J=8.2 Hz, 1H), 7.34 (dd, J=8.2, 1.8 Hz, 1H), 7.26-7.01 (m, 6H), 5.57 (s, 2H), 3.86 (t, J=6.5 Hz, 2H), 2.56 (d, J=7.2 Hz, 2H), 2.06-1.75 (m, 1H), 1.54 (s, 9H), 1.49-1.35 (m, 2H), 1.34-1.16 (m, 2H), 0.92 (d, J=6.6 Hz, 6H), 0.85 (t, J=7.4 Hz, 3H). ¹³C NMR (101 MHz, Methanol-d₄) δ 160.8 (d, ¹J_{C-F}=246.6 Hz), 156.8, 154.9, 147.6, 144.3 (d, ³J_{C-F}=8.6 Hz), 140.0, 138.8, 133.6, 130.7, 129.9 (d, ⁴J_{C-F}=4.0 Hz), 129.7, 127.0 (d, ³J_{C-F}=3.3 Hz), 124.1, 123.0 (d, ²J_{C-F}=14.6 Hz), 122.9, 117.9 (d, ²J_{C-F}=22.0 Hz), 66.3, 47.3 (d, ³J_{C-F}=4.1 Hz), 45.8, 34.7, 32.0, 31.2, 29.4, 22.7, 20.0, 14.1. ¹⁹F NMR (376 MHz, Methanol-d₄) δ −113.55-−127.68 (m).

### EXAMPLE 50

### Methyl (4′-((2-(tert-butyl)-1H-imidazol-1-yl)methyl)-3′-fluoro-5-isobutyl-[1,1′-biphenyl]-2-yl)sulfonylcarbamate

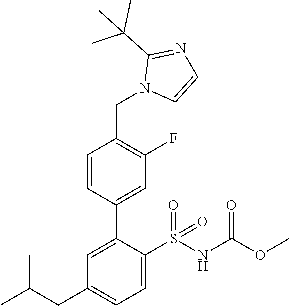 [SMILES: COC(=O)NS(=O)(=O)c1ccc(CC(C)C)cc1-c1ccc(Cn2ccnc2C(C)(C)C)c(F)c1]

The title compound was synthesized as described for methyl ((4′-((2-(tert-butyl)-1H-imidazol-1-yl)methyl)-5-propyl-[1,1′-biphenyl]-2-yl)sulfonyl)carbamate using the corresponding butyl carbamate (33.0 mg, 60.7 μmol) in MeOH (1 mL). The crude product was concentrated and purified by FCC (5% MeOH in CH₂Cl₂) to afford the product as a white amorphous solid (17.5 mg, 58% yield). ¹H NMR (400 MHz, Methanol-d₄) δ 8.05 (d, J=8.2 Hz, 1H), 7.34 (dt, J=8.2, 1.7 Hz, 1H), 7.27-7.14 (m, 4H), 7.13-7.03 (m, 2H), 5.58 (s, 2H), 3.45 (s, 3H), 2.57 (d, J=7.1 Hz, 2H), 2.06-1.77 (m, J=6.7 Hz, 1H), 1.55 (s, 9H), 0.93 (d, J=6.6 Hz, 6H). ¹³C NMR^{a} (101 MHz, Methanol-d₄) δ 160.6 (d, ¹J_{C-F}=246.7 Hz), 157.5, 154.6, 147.2, 144.2 (d, ³J_{C-F}=8.5 Hz), 139.7, 138.6, 133.3, 130.5, 129.5 (d, ⁴J_{C-F}=2.3 Hz), 126.8 (d, ³J_{C-F}=3.3 Hz), 123.9, 122.7, 122.5 (d, ²J_{C-F}=14.4 Hz), 117.8 (d, ²J_{C-F}=22.0 Hz), 52.8, 47.1 (d, ³JC-F=4.0 Hz), 45.7, 34.5, 31.0, 29.3, 22.6. ¹⁹F NMR (376 MHz, Methanol-d₄) δ −116.33-−132.46 (m).

Biology

Results

Kᵢ values, stability in liver microsomes and kinetic solubility

Kᵢ determinations. The compounds were evaluated in a radioagonist assay by displacing [¹²⁵I][Sar¹Ile⁸]-angiotensin II from human AT2R in HEK-293 cells membrane preparations at Eurofins. [Sar¹Ile⁸]-angiotensin II (Sarile) acts as a nonselective AT2R agonist.¹⁸ The affinity was determined using an eight- or nine-point dose-response curve, each point performed in duplicates. The incubation buffer (final concentrations) consisted of 45 mMTris/HCI, 4.5 mM MgCl₂, 0.9 mM EDTA/Tris, 0.09% BSA at pH 7.4. PD 123,319 was used as a reference compound. The compounds were also evaluated for inhibition of [¹²⁵I][Sar¹Ile⁸]-angiotensin II binding to human AT1R in HEK-293 cell membranes at Eurofins using Saralasin as the reference compound.

Stability in liver microsomes. Human, mouse and rat liver microsomes were used to assess the metabolic stability for the compounds. Metabolic stability was determined in 0.5 mg/mL human, mouse or rat liver microsomes at a compound concentration of 1 μM in 100 mM potassium phosphate buffer (pH 7.4) in a total incubation volume of 500 μL by Eurofins. The assay matrix was: human liver microsomes: mixed gender and pool of 50, rat liver microsomes: male, Sprague-Dawley, pool of 100 or more and mouse liver microsomes: male,CD-1, pool of 250 or more. Final microsomal protein concentration: 0.1mg/mL. The test concentration was 0.1 μM with 0.01% DMSO, 0.25% acetonitrile and 0.25% methanol by default. The experimental protocol was as following: the test compound was pre-incubated with pooled liver microsomes in phosphate buffer (pH 7.4) for 5 min in a 37° C. shaking waterbath. The reaction was initiated by adding NADPH-generating system and was incubated for 0, 15, 30, 45, and 60 min. The reaction was stopped by transferring the incubation mixture to acetonitrile/methanol. Samples were then mixed and centrifuged. Four reference compounds were tested in each assay. Propranolol and imipramine are relatively stable, whereas verapamil and terfenadine are readily metabolized inhuman liver microsomes. Samples were analyzed by HPLC-MS/MS using selected reaction monitoring. The HPLC system consisted of a binary LC pump with autosampler, a C-18 column, and a gradient. Peak areas corresponding to the test compound were recorded. The compound remaining was calculated by comparing the peak area at each time point to time zero. The half-life was calculated from the slope of the initial linear range of the logarithmic curve of compound remaining (%) vs. time, assuming first order kinetics. In addition, the intrinsic clearance (Clint) was calculated.^{19,20}

Kinetic solubility. Dulbecco's PBS buffer was used (NaCl 137 mM, KCl 2.7 mM, Na₂HPO₄ 8.1 mM, KH₂PO₄ 1.5 mM with pH 7.4). The test compound is prepared at 200 μM in the PBS buffer from a 10 mM DMSO stock solution. The final DMSO concentration is 2%. The buffer samples were mixed thoroughly followed by incubation at room temperature for 24 h. At the end of the incubation, the buffer samples were centrifuged and supernatants were then injected for the HPLC analysis. A calibration standard of the test compound was prepared at 200 μM in methanol/water (3/2, v/v) from a 10 mM DMSO stock solution on the day of HPLC analysis. The analysis was conducted by HPLC-UV/VIS with photodiode array detection, with monitoring at 205, 230, 260, and 300 nm wavelengths. The HPLC system consisted of a C18 column using a gradient of A and B (A=12 mM ammonium formate, 6 mM formic acid in water, pH 4.0, B=6 mM ammonium formate, 3 mM formic acid acetonitrile/water (9/1, v/v)). Reference compounds used were metoprolol, rifampicin, ketoconazole, phenytoin, haloperidol, simvastatin, diethylstilbestrol, and tamoxifen, ranking from fully soluble (200 μM) to poorly soluble (<1μM). The aqueous solubility (μM) of the test compound was determined by comparing the peak area of the principal peak in the calibration standard (200 μM) with the peak area of the corresponding peak in each of the buffer samples. The range of the assay was approximately 0.5 μM to 200 μM. The chromatographic purity (%) was determined as the peak area of the principal peak relative to the total integrated peak areas in the HPLC chromatogram of the calibration standard.

Table 1 below shows Kᵢ and stability in human liver microsomes (HLM), mouse liver microsomes (MLM) and rat liver microsomes. (RLM) and the kinetic stability in PBS for the compounds of the indicated examples.

TABLE 1

| | | | | | | Kinetic |
|---|---|---|---|---|---|---|
| | | | Stability in liver | | | solubility |
| Example | Ki [nM] | | microsomes [min] | | | PBS pH 7.4 |
| number | AT₁R | AT₂R | HLM | MLM | RLM | [μM] |
| Example 1A | ~1000 | 1.1 | >60 | >60 | >60 | 140 |
| Example 2A | ~10.000 | 15 | | | | |
| Example 2B | | | 11 | 10 | 8 | 183 |
| Example 3 | ~1000 | 29 | >60 | >60 | >60 | 195 |
| Example 4 | ~1000 | 18 | 45 | 7 | 12 | 180 |
| Example 5 | ~1000 | 190 | | | | |
| Example 6 | ~1000 | 71 | | | | |
| Example 7B | ~1000 | 5.1 | 28 | 20 | 22 | >200 |
| Example 8B | ~1000 | 3.2 | >60 | >60 | 41 | >200 |
| Example 9B | ~1000 | 4.7 | | | | |
| Example 10B | ~1000 | 0.97 | | | | |
| Example 11B | ~1000 | 6.6 | 11 | 10 | 18 | >200 |
| Example 12B | ~1000 | 3.3 | >60 | 39 | >60 | >200 |
| Example 13 | >10.000 | 16 | 36 | 39 | 20 | 198 |
| (i.e. sodium | | | | | | |
| salt of C21) | | | | | | |
| Example 14 | 10.000 | 160 | | | | |
| Example 15 | 10.000 | 39 | >60 | | | |
| Example 16 | 1000 | 41 | >60 | | | |
| Example 17A | 1000 | 12 | >60 | | | |
| Example 18 | 1000 | 5.8 | >60 | | | |
| Example 19 | 10.000 | 200 | | | | |
| Example 20 | 10.000 | 27 | >60 | | | |
| Example 21 | 1000 | 350 | | | | |
| Example 22 | 10.000 | 540 | | | | |
| Example 23 | 10.000 | 27 | >60 | | | |
| Example 24 | 1000 | 250 | | | | |
| Example 25 | 10.000 | 1600 | | | | |
| Example 26 | 13000 | 99 | | | | |
| Example 27 | 8000 | 11 | | | | |
| Example 28 | 20.000 | 274 | | | | |
| Example 29 | 15000 | 36 | | | | |
| Example 30B | 1000 | 4.6 | >60 | | | |
| Example 31B | 1000 | 1.1 | 20 | | | |
| Example 32 | 10.000 | 1 | | | | |
| Example 33B | 8000 | 6 | | | | |
| Example 34 | 15000 | 2.3 | | | | |
| Example 35B | 7000 | 6.7 | | | | |
| Example 36 | | 3.26 | | | | |
| Example 37A | | 2.22 | | | | |
| Example 38A | | 5.64 | | | | |
| Example 39A | | 4.8 | | | | |
| Example 40A | | 9.62 | | | | |
| Example 41 | | 3.08 | | | | |
| Example 42 | | 1.66 | | | | |
| Example 43 | | 3.74 | | | | |
| Example 44 | | 2.54 | | | | |
| Example 45 | | >1000 | | | | |
| Example 46 | | >1000 | | | | |
| Example 47 | | 56 | | | | |
| Example 48 | | 475 | | | | |

CYP450 Inhibition Assays

The following procedure was designed to assess if a test compound inhibits the activity of each of the common cytochrome P450 (CYP) enzymes in pooled human liver microsomes in 96-well plate format. The compound was tested at a single concentration (10 μM) with 0.1% DMSO. The test compound was pre-incubated with substrate and human liver microsomes (mixed gender, pool of 50 donors, 0.1mg/mL) in phosphate buffer (pH 7.4) for 5 min in a 37 C. shaking waterbath. The reaction was initiated by adding a NADPH-generating system. The reaction was allowed for 10 min and stopped by transferring the reaction mixture to acetonitrile/methanol. Samples were mixed and centrifuged. Supernatants were used for HPLC-MS/MS of the respective metabolite. The activity of human cytochrome P450 (CYP) enzymes were determined by following the formation of a metabolite of the probe substrate. The following enzymes were evaluated for inhibition using the listed reference substrates and reference inhibitors:

CYP1A—phenacetin (reference inhibitor furafylline), CYP2B6—bupropion (reference inhibitor clopidogre), CYP2C8-amodiaquine (reference inhibitor montelukast), CYP2C9—diclofenac (reference inhibitor sulfaphenazole), CYP2C19—omeprazole (reference inhibitor oxybutynin), CYP2D6—dextromethorphan (reference inhibitor quinidine), CYP3A—midazolam (reference inhibitor ketoconazol) and CYP3A—testosterone (reference inhibitor ketoconazol). The reference inhibitors were tested in each assay at multiple concentrations to obtain an IC50 value. Peak areas corresponding to the metabolite were recorded. The percent of control activity was calculated by comparing the peak area in the presence of the test compound to the control samples containing the same solvent. Subsequently, the percent inhibition was calculated by subtracting the percent control activity from 100. The IC50 value (concentration causing a half-maximal inhibition of the control value) was determined by non-linear regression analysis of the concentration-response curve using the Hill equation.

TABLE 2
CYP 450 Inhibition (% at 10 μM) for Example
44 (the sodium salt of C21) and other Examples

| Example | | | | | | |
|---|---|---|---|---|---|---|
| number | CYP1A | CYP2B6 | CYP2C8 | CYP2C9 | CYP2C19 | CYP2D6 |
| C21* | 63 | 40 | | 94 | 67 | 41 |
| Example 13 | 77 | 53 | 69 | 99 | 93 | 90 |
| (i.e. sodium | | | | | | |
| salt of C21) | | | | | | |
| Example 13 | 89 | 63 | 82 | 99 | 96 | 90 |
| (i.e. sodium | | | | | | |
| salt of C21) | | | | | | |
| Example 1A | 11 | 18 | 67 | 37 | 38 | 9 |
| Example 2B | 4.5 | 57 | 81 | 91 | 71 | 15 |
| Example 3 | 8.4 | 24 | 26 | 23 | 38 | 12 |
| Example 4 | −7.3 | 29 | 70 | 68 | 66 | 12 |
| Example 7B | −50 | 40 | 68 | 74 | 52 | 16 |
| Example 8B | 5.7 | 28 | 64 | 92 | 45 | 2.5 |
| Example 11B | 8.4 | 51 | 81 | 88 | 66 | 14 |
| Example 12B | 8.3 | 25 | 73 | 29 | 36 | 1.6 |
| Example 32 | −3.04 | 29.76 | 66.46 | 43.71 | 31.01 | 17.05 |
| Example 34 | −5.97 | 6.90 | 70.26 | 42.16 | 43.29 | 16.22 |

| Example | CYP3A | CYP3A |
|---|---|---|
| number | midazolam | testosterone |
| C21* | 82 | |
| Example 13 | 93 | 84 |
| (i.e. sodium | | |
| salt of C21) | | |
| Example 13 | 93 | 90 |
| (i.e. sodium | | |
| salt of C21) | | |
| Example 1A | 0 | 14 |
| Example 2B | 16 | 27 |
| Example 3 | 5.4 | 2.7 |
| Example 4 | 24 | 7.5 |
| Example 7B | −10.7 | 3.1 |
| Example 8B | 1.3 | 10.7 |
| Example 11B | −5.1 | 21 |
| Example 12B | −11.6 | 6.8 |
| Example 32 | −11.70 | 8.43 |
| Example 34 | 9.98 | 18.08 |

*Bioorg. Med. Chem. 2010, 18 (12), 4570-4590.

Testing on Human IPF Precesion-Cut Lung Slices (PCLuS)

The following procedure was performed by FibroFind (Newcastle Fibrosis Research Group, United Kingdom) and was designed to assess if a test compound attenuates fibrosis in IPF-PCLuS (IPF Precision Cut Lung Slices). The PCLus were prepared from biopsy confirmed, explanted IPF human lung tissue collected at the time of lung transplantation. PCLus were then rested for 48 hours to allow the post-slicing stress period to elapse before experiments began. In addition, PCLuS were cultured in the presence of the test compounds at two escalating doses (0.1 μM and 1 μM). All PCLuS were harvested at 144 hrs.

Nine different groups with n=5 or 8 human PCLuS were investigated as shown in Table 3 below. PCLuS were prepared from n=1 biopsy confirmed, explanted human IPF lung. Vehicle is DMSO diluted 1:1000 in the culture medium.

TABLE 3

| | | | | Intervention | |
|---|---|---|---|---|---|
| Group | Intervention | Dose | Rest | duration | PCLuS |
| 1 | Vehicle | — | 48 hrs | 48 to 144 hrs | n = 12 |
| 3 | C21 | 0.1 μM | 48 hrs | 48 to 144 hrs | n = 5 |
| 4 | C21 | 1 μM | 48 hrs | 48 to 144 hrs | n = 5 |
| 5 | Example 1A | 0.1 μM | 48 hrs | 48 to 144 hrs | n = 5 |
| 6 | Example 1A | 1 μM | 48 hrs | 48 to 144 hrs | n = 5 |
| 7 | Example 12A | 0.1 μM | 48 hrs | 48 to 144 hrs | n = 5 |
| 8 | Example 12A | 1 μM | 48 hrs | 48 to 144 hrs | n = 5 |
| 8 | Example 3 | 0.1 μM | 48 hrs | 48 to 144 hrs | n = 5 |
| 9 | Example 3 | 1 μM | 48 hrs | 48 to 144 hrs | n = 5 |
| | | | | Total PCLuS | n = 52 |

PCLuS were incubated for a 48 hr rest period. Post-rest, PCLuS were incubated for a further 96hrs in the presence or absence of the test compounds as outlined above. PCLuS culture media, including all test compounds, were refreshed and harvested at 24 hrs intervals from 48 hrs. All PCLuS were harvested at 144 hrs. Each media sample was split into 3 aliquots of ˜150 ul, with a single aliquot used for the measurement of soluble markers. The soluble markers are Collagen 1a1 (Col1a1) and transforming growth factor-β1 (TGF-β1).

PCLuS harvest. Cell culture supernatant (n=5 to 12 per group) were collected daily and snap frozen for quantification of the soluble outputs listed below. At harvest, n=5 to 8 PCLuS were snap frozen for RNA isolation and qPCR analysis.

Soluble outputs analysis. Levels of Collagen 1a1 in the cell culture supernatants were quantified using R&D Duoset ELISA kits. Levels of TGF-β1 in the cell culture supernatants were quantified using a SinglePlex ELISA (MesoScaleDiscovery™ (MSD)).

Quantitative real time PCR (qPCR). Total RNA extraction from PCLuS was performed on all samples using a MagMAX™-96 Total RNA Isolation Kit. RNA was reverse transcribed to cDNA and used in qPCRs to measure transcript levels of Col1a1, TGF-β1 and β-Actin.

Soluble outputs analysis. Levels of Col1a1 in the cell culture supernatant were quantified at 48 hrs, 96 hrs and 144 hrs using an R&D Duoset ELISA kit, while levels of soluble TGF-β1 were quantified using a SinglePlex ELISA MSD kit (FIGS. 1 to 8).

Quantitative real time PCR. Transcript levels of Col1a1 and TGF-β1 in PCLuS were quantified relative to β-Actin (FIGS. 9 and 10).

FIGS. 1 to 4 show the secreted Collagen 1a1 levels in PCLus culture supernatants when treated with C21, Example 1A, Example 12A and Example 3. The graphs are represented by n=5 to 12 tissue culture media per condition, per time point. Data is presented as mean±SEM with individual values per tissue culture media.

FIGS. 5 to 8 show the secreted TGF-β1 levels in PCLus culture supernatants when treated with C21, Example 1A, Example 12A and Example 3. The graphs are represented by n=5 to 12 tissue culture media per condition, per time point. Data is presented as mean±SEM with individual values per tissue culture media.

FIGS. 9 and 10 show selected gene transcript levels in PCLus in the form of the relative level of transcriptional difference (RLTD %) for Colla 1 and TGF-β1 (normalised to (β-Actin) with C21, Example 1A, Example 12A and Example 3 treatment at 144 h. The data is presented as percentage change in gene expression relative to control and vehicle.

Treatment of IPF PCLus with virtually all compounds tested robustly reduced TGF-β1 gene transcription and protein secretion, at least at one time point tested. These compounds were also very efficacious at reducing Collagen 1a1 gene transcription.

### Further Examples

There is also provided the following compounds, which may be prepared in accordance with one or more of the synthetic procedures described hereinbefore.

TABLE 3
Prophetic Examples

| Prophetic | | |
|---|---|---|
| Example No. | Chemical structure | Chemical name |
| 1 | 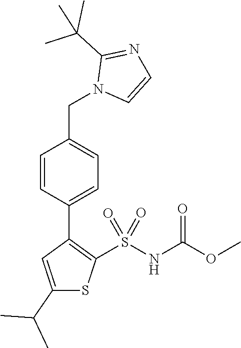 [SMILES: COC(=O)NS(=O)(=O)c1sc(C(C)C)cc1-c1ccc(Cn2ccnc2C(C)(C)C)cc1] | Methyl ((3-(4-((2-(tert-butyl)- 1H-imidazol-1- yl)methyl)phenyl)-5- isopropylthiophen-2- yl)sulfonyl)carbamate |
| 2 | 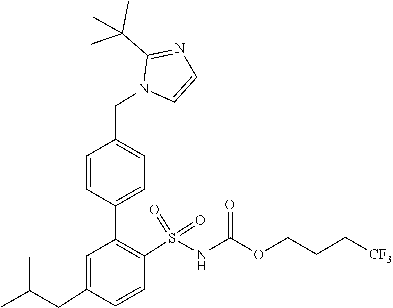 [SMILES: CC(C)Cc1ccc(S(=O)(=O)NC(=O)OCCCC(F)(F)F)c(-c2ccc(Cn3ccnc3C(C)(C)C)cc2)c1] | 4,4,4-Trifluorobutyl ((4'-((2- ('tert-butyl)-1H-imidazol-1- yl)methyl)-5-isobutyl-[1,1'- biphenyl]-2- yl)sulfonyl)carbamate |
| 3 | 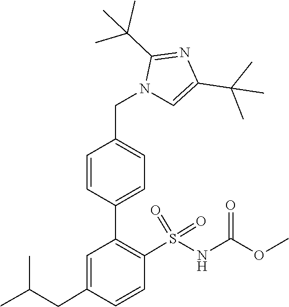 [SMILES: COC(=O)NS(=O)(=O)c1ccc(CC(C)C)cc1-c1ccc(Cn2cc(C(C)(C)C)nc2C(C)(C)C)cc1] | Methyl((4‘-((2,4-di-tert- butyl-1H-imidazol-1- yl)methyl)-5-isobutyl-[1,1'- biphenyl]-2- yl)sulfonyl)carbamate |
| 4 | 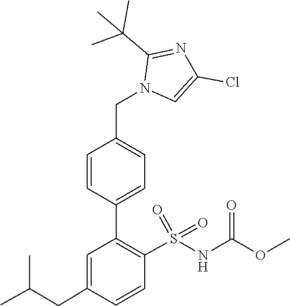 [SMILES: COC(=O)NS(=O)(=O)c1ccc(CC(C)C)cc1-c1ccc(Cn2cc(Cl)nc2C(C)(C)C)cc1] | Methyl ((4‘-((2-(tert-butyl)-4- chloro-1H-imidazol-1- yl)methyl)-5-isobutyl-[1,1'- biphenyl]-2- yl)sulfonyl)carbamate |
| 5 | 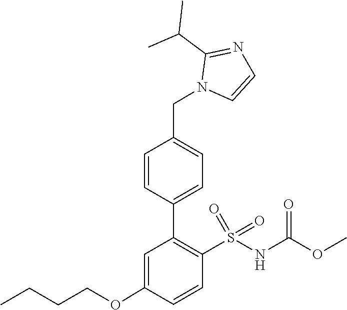 [SMILES: CCCCOc1ccc(S(=O)(=O)NC(=O)OC)c(-c2ccc(Cn3ccnc3C(C)C)cc2)c1] | Methyl((5-butoxy-4'-((2- isopropyl-1H-imidazol-1- yl) methyl)-[1,1'-biphenyl]-2- yl)sulfonyl)carbamate |
| 6 | 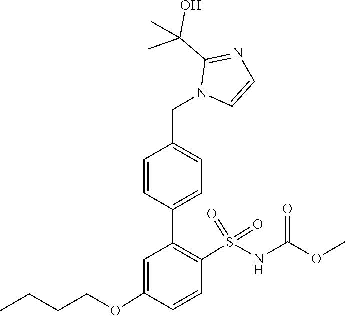 [SMILES: CCCCOc1ccc(S(=O)(=O)NC(=O)OC)c(-c2ccc(Cn3ccnc3C(C)(C)O)cc2)c1] | Methyl ((5-butoxy-4'-((2-(2- hydroxypropan-2-yl)-1H- imidazol-1-yl)methyl)-[1,1'- biphenyl]-2- yl)sulfonyl)carbamate |
| 7 | 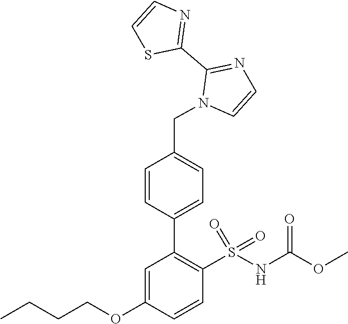 [SMILES: CCCCOc1ccc(S(=O)(=O)NC(=O)OC)c(-c2ccc(Cn3ccnc3-c3nccs3)cc2)c1] | Methyl((5-butoxy-4‘-((2- (thiazol-2-yl)-1H-imidazol-1- yl) methyl)-[1,1'-biphenyl]-2- yl)sulfonyl)carbamate |
| 8 | 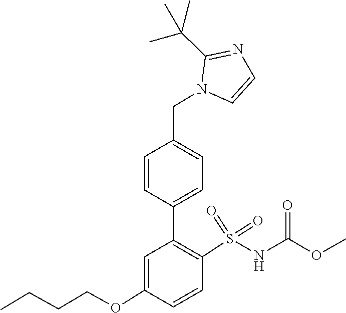 [SMILES: CCCCOc1ccc(S(=O)(=O)NC(=O)OC)c(-c2ccc(Cn3ccnc3C(C)(C)C)cc2)c1] | Methyl((5-butoxy-4'-((2- ('tert-butyl)-1H-imidazol-1- yl) methyl)-[1,1'-biphenyl]-2- yl)sulfonyl)carbamate |
| 9 | 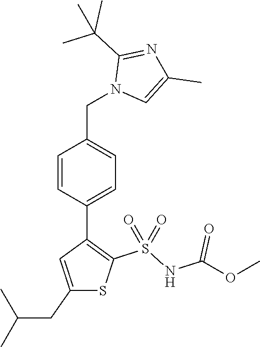 [SMILES: COC(=O)NS(=O)(=O)c1sc(CC(C)C)cc1-c1ccc(Cn2cc(C)nc2C(C)(C)C)cc1] | Methyl ((3-(4-((2-(tert-butyl)- 4-methyl-1H-imidazol-1- yl)methyl)phenyl)-5- isobutylthiophen-2- yl)sulfonyl)carbamate |
| 10 | 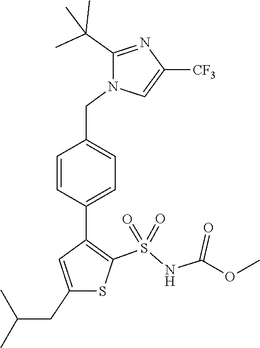 [SMILES: COC(=O)NS(=O)(=O)c1sc(CC(C)C)cc1-c1ccc(Cn2cc(C(F)(F)F)nc2C(C)(C)C)cc1] | Methyl ((3-(4-((2-(tert-butyl)- 4-(trifluoromethyl)-1H- imidazol- 1-yl) methyl) phenyl)- 5-isobutylthiophen-2- yl)sulfonyl)carbamate |
| 11 | 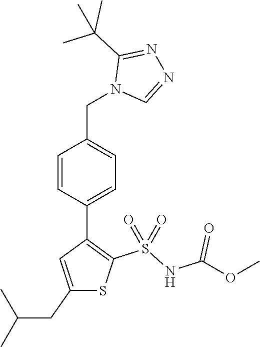 [SMILES: COC(=O)NS(=O)(=O)c1sc(CC(C)C)cc1-c1ccc(Cn2cnnc2C(C)(C)C)cc1] | Methyl ((3-(4-((3-(tert-butyl)- 4H-1,2,4-triazol-4- yl)methyl)phenyl)-5- isobutylthiophen-2- yl)sulfonyl)carbamate |
| 12 | 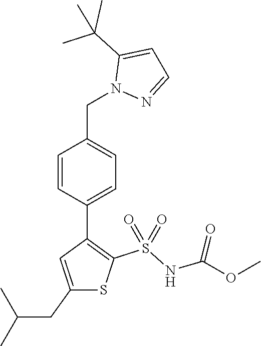 [SMILES: COC(=O)NS(=O)(=O)c1sc(CC(C)C)cc1-c1ccc(Cn2nccc2C(C)(C)C)cc1] | Methyl ((3-(4-((5-(tert-butyl)- 1H-pyrazol-1- yl)methyl)phenyl)-5- isobutylthiophen-2- yl)sulfonyl)carbamate |
| 13 | 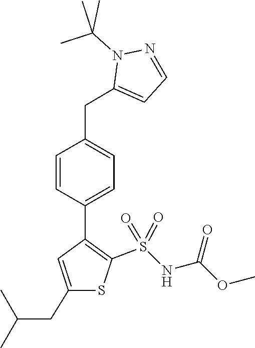 [SMILES: COC(=O)NS(=O)(=O)c1sc(CC(C)C)cc1-c1ccc(Cc2ccnn2C(C)(C)C)cc1] | Methyl ((3-(4-((1-(tert-butyl)- 1H-pyrazol-5- yl)methyl)phenyl)-5- isobutylthiophen-2- yl)sulfonyl)carbamate |
| 14 | 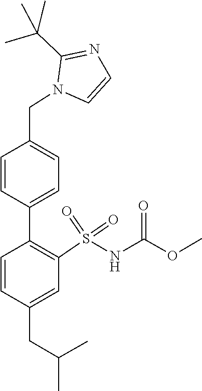 [SMILES: COC(=O)NS(=O)(=O)c1cc(CC(C)C)ccc1-c1ccc(Cn2ccnc2C(C)(C)C)cc1] | Methyl ((4‘-((2-(tert-butyl)- 1H-imidazol-1-yl)methyl)-4- isobutyl-[1,T-biphenyl]-2- yl)sulfonyl)carbamate |
| 15 | 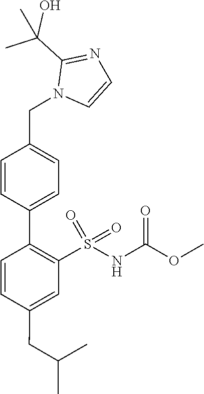 [SMILES: COC(=O)NS(=O)(=O)c1cc(CC(C)C)ccc1-c1ccc(Cn2ccnc2C(C)(C)O)cc1] | Methyl((4‘-((2-(2- hydroxypropan-2-yl)-1H- imidazol-1-yl)methyl)-4- isobutyl-[1,1'-biphenyl]-2- yl)sulfonyl)carbamate |
| 16 | 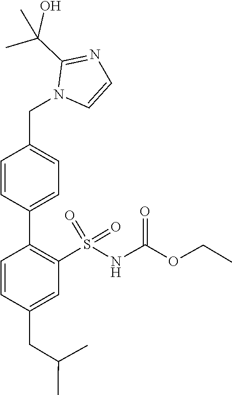 [SMILES: CCOC(=O)NS(=O)(=O)c1cc(CC(C)C)ccc1-c1ccc(Cn2ccnc2C(C)(C)O)cc1] | Ethyl((4-((2-(2- hydroxypropan-2-yl)-1H- imidazol-1-yl)methyl)-4- isobutyl-[1,1'-biphenyl]-2- yl)sulfonyl)carbamate |
| 17 | 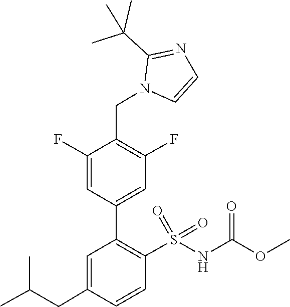 [SMILES: COC(=O)NS(=O)(=O)c1ccc(CC(C)C)cc1-c1cc(F)c(Cn2ccnc2C(C)(C)C)c(F)c1] | Methyl ((4‘-((2-(tert-butyl)- 1H-imidazol-1-yl) methyl)- 3',5'-difluoro-5-isobutyl-[1,1'- biphenyl]-2- yl)sulfonyl)carbamate |
| 18 | 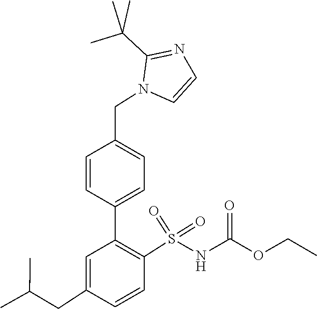 [SMILES: CCOC(=O)NS(=O)(=O)c1ccc(CC(C)C)cc1-c1ccc(Cn2ccnc2C(C)(C)C)cc1] | Ethyl ((4‘-((2-(tert-butyl)-1H- imidazol-1-yl)methyl)-5- isobutyl-[1,1'-biphenyl]-2- yl)sulfonyl)carbamate |
| 19 | 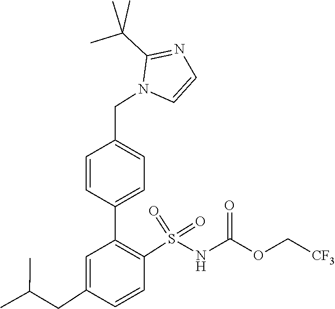 [SMILES: CC(C)Cc1ccc(S(=O)(=O)NC(=O)OCC(F)(F)F)c(-c2ccc(Cn3ccnc3C(C)(C)C)cc2)c1] | 2,2,2-Trifluoroethyl ((4‘-((2- ('tert-butyl)-1H-imidazol-1- yl)methyl)-5-isobutyl-[1,1'- biphenyl]-2- yl)sulfonyl)carbamate |
| 20 | 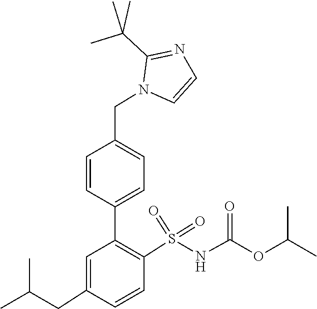 [SMILES: CC(C)Cc1ccc(S(=O)(=O)NC(=O)OC(C)C)c(-c2ccc(Cn3ccnc3C(C)(C)C)cc2)c1] | Isopropyl((4‘-((2-(tert- butyl)-1H-imidazol-1- yl)methyl)-5-isobutyl-[1,1'- biphenyl]-2- yl)sulfonyl)carbamate |
| 21 | 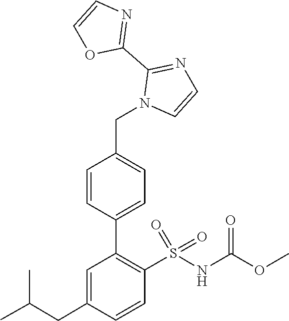 [SMILES: COC(=O)NS(=O)(=O)c1ccc(CC(C)C)cc1-c1ccc(Cn2ccnc2-c2ncco2)cc1] | Methyl ((5-isobutyl-4'-((2- (oxazol-2-yl)-1H-imidazol-1- yl) methyl)-[1,1'-biphenyl]-2- yl)sulfonyl)carbamate |
| 22 | 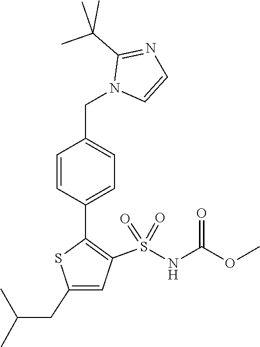 [SMILES: COC(=O)NS(=O)(=O)c1cc(CC(C)C)sc1-c1ccc(Cn2ccnc2C(C)(C)C)cc1] | Methyl ((2-(4-((2-(tert-butyl)- 1H-imidazol-1- yl)methyl)phenyl)-5- isobutylthiophen-3- yl)sulfonyl)carbamate |
| 23 | 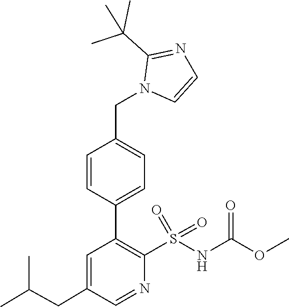 [SMILES: COC(=O)NS(=O)(=O)c1ncc(CC(C)C)cc1-c1ccc(Cn2ccnc2C(C)(C)C)cc1] | Methyl ((3-(4-((2-(tert-butyl)- 1H-imidazol-1- yl)methyl)phenyl)-5- isobutylpyridin-2- yl)sulfonyl)carbamate |
| 24 | 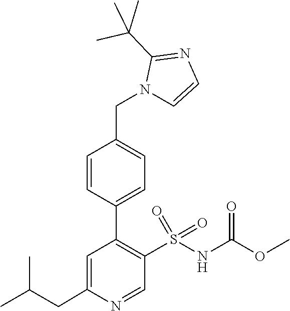 [SMILES: COC(=O)NS(=O)(=O)c1cnc(CC(C)C)cc1-c1ccc(Cn2ccnc2C(C)(C)C)cc1] | Methyl ((4-(4-((2-(tert-butyl)- 1H-imidazol-1- yl)methyl)phenyl)-6- isobutylpyridin-3- yl)sulfonyl)carbamate |
| 25 | 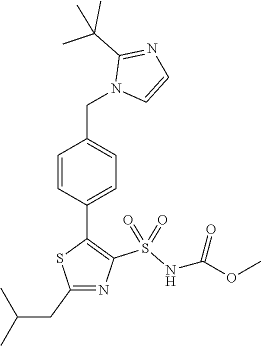 [SMILES: COC(=O)NS(=O)(=O)c1nc(CC(C)C)sc1-c1ccc(Cn2ccnc2C(C)(C)C)cc1] | Methyl ((5-(4-((2-(tert-butyl)- 1H-imidazol-1- yl)methyl)phenyl)-2- isobutylthiazol-4- yl)sulfonyl)carbamate |
| 26 | 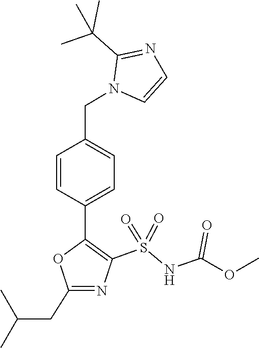 [SMILES: COC(=O)NS(=O)(=O)c1nc(CC(C)C)oc1-c1ccc(Cn2ccnc2C(C)(C)C)cc1] | Methyl ((5-(4-((2-(tert-butyl)- 1H-imidazol-1- yl)methyl)phenyl)-2- isobutyloxazol-4- yl)sulfonyl)carbamate |
| 27 | 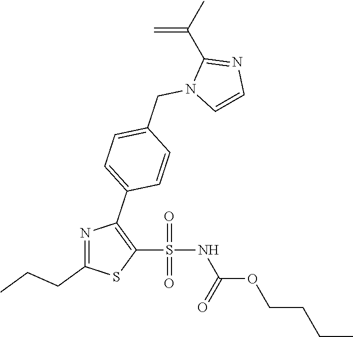 [SMILES: C=C(C)c1nccn1Cc1ccc(-c2nc(CCC)sc2S(=O)(=O)NC(=O)OCCCC)cc1] | Butyl (4-(4-((2-(prop-1-en-2- yl)-1H-imidazol-1- yl)methyl)phenyl)-2- propylthiazol-5- yl)sulfonylcarbamate |
| 28 | 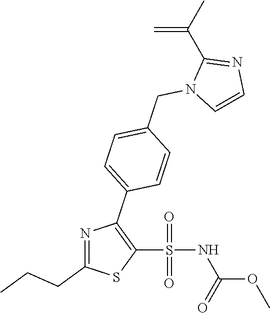 [SMILES: C=C(C)c1nccn1Cc1ccc(-c2nc(CCC)sc2S(=O)(=O)NC(=O)OC)cc1] | Methyl (4-(4-((2-(prop-1-en- 2-yl)-1H-imidazol-1- yl)methyl)phenyl)-2- propylthiazol-5- yl)sulfonylcarbamate |
| 29 | 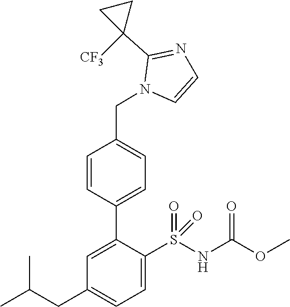 [SMILES: COC(=O)NS(=O)(=O)c1ccc(CC(C)C)cc1-c1ccc(Cn2ccnc2C2(C(F)(F)F)CC2)cc1] | Methyl (5-isobutyl-4'-((2-(l- (trifluoromethyl)cyclopropyl)- 1H-imidazol-1-yl)methyl)- [1,T-biphenyl]-2- yl)sulfonylcarbamate |

### CONCLUSIONS

In conclusion, in comparison to the compound C21, or the sodium salt thereof, the compounds of the present disclosure, or salts thereof, were found to exhibit low inhibition of CYP enzymes.

### REFERENCES

1. Pharmacol. Rev. 2000; 52: 415-472.

2. WO 02/096883

3. WO 99/43339

4. EP 0512675 A1

5. DE10 2012 004 589 A1

6. US 2004/0167176

7. Med. Res. Rev. 2018; 38:602-624

8. WO 2002/096883

9. WO 2016/139475

10. WO 2004/046141

11. WO 2016/092329

12. WO 2016/107879

13. WO 2016/139475

14. WO 2017/221012

15. WO 2019/008393

16. US 2012/035232

17. Bioorg. Med. Chem. Lett. 2018, 28 (3), 519-522.

18. ACS Med. Chem. Lett. 2014, 5, 1129-1132.

19. Biochem. Pharmacol. 1994, 47, 1469-1479.

20. DRUG Metab. Dispos. 1999, 27, 1350-1359.

21. Bioorg. Med. Chem. 2010, 18 (12), 4570-4590.

1. A compound of Formula I: 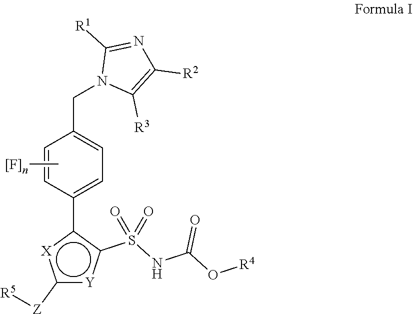
or a pharmaceutically acceptable salt thereof,
wherein
R¹ represents
H,
C₂-C₆ alkyl substituted with 0, 1 or 2 substituents selected from the group consisting of OR⁶, SR⁷, NR⁸R⁹, halogen, thiazole, oxazole and pyrazole, C₃-C₆ cycloalkyl substituted with 0, 1 or 2 substituents selected from the group consisting of OR⁶, SR⁷ and NR⁸R⁹,
thiazole,
benzyl wherein the phenyl moiety is substituted with 0, 1 or 2 substituents selected from the group consisting of OR⁶, SR⁷ and NR⁸R⁹, or
(CH₂)ₘ—R¹⁰,
R² represents
H,
F,
Cl,
C₂-C₆ alkyl substituted with 0, 1 or 2 substituents selected from the group consisting of OR⁶, SR⁷, NR⁸R⁹, halogen, thiazole, oxazole and pyrazole,
C₃-C₆ cycloalkyl substituted with 0, 1 or 2 substituents selected from the group consisting of OR⁶, SR⁷ and NR⁸R⁹,
benzyl wherein the phenyl moiety is substituted with 0, 1 or 2 substituents selected from the group consisting of OR⁶, SR⁷ and NR⁸R⁹, or
(CH₂)ₘ—R¹⁰,
provided that R¹ and R² are not both H,
R³ represents
H,
halogen, or
C₁-C₃ alkyl substituted with 0, 1, 2 or 3 halogens selected from the group consisting of F and Cl,
R⁴ and R⁵ independently represent C₁-C₆alkyl substituted with 0, 1, 2 or 3 F,
X represents CH═CH, CH, N, NH, O or S;
Y represents CH═CH, CH, N, NH, O or S,
provided that:
(a) X and Y are not the same,
(b) when X represents CH═CH then Y may only represent CH, and
(c) when Y represents CH═CH then X may only represent CH,
Z represents a single bond, O or S,
R⁶, R⁷, R⁵ and R⁹ independently represent
H, or
C₁-C₃ alkyl substituted with 0,1, 2 or 3 F,
R¹⁰ is selected from the group consisting of phenyl, thiazole, oxazole and pyrazole,
n is 0, 1, 2, 3 or 4, and
m is 0 or 1.

2. A compound as claimed in claim 1, or a pharmaceutically acceptable salt thereof, which is a compound of Formula Ia, Formula Ib, Formula Ic or Formula Id:

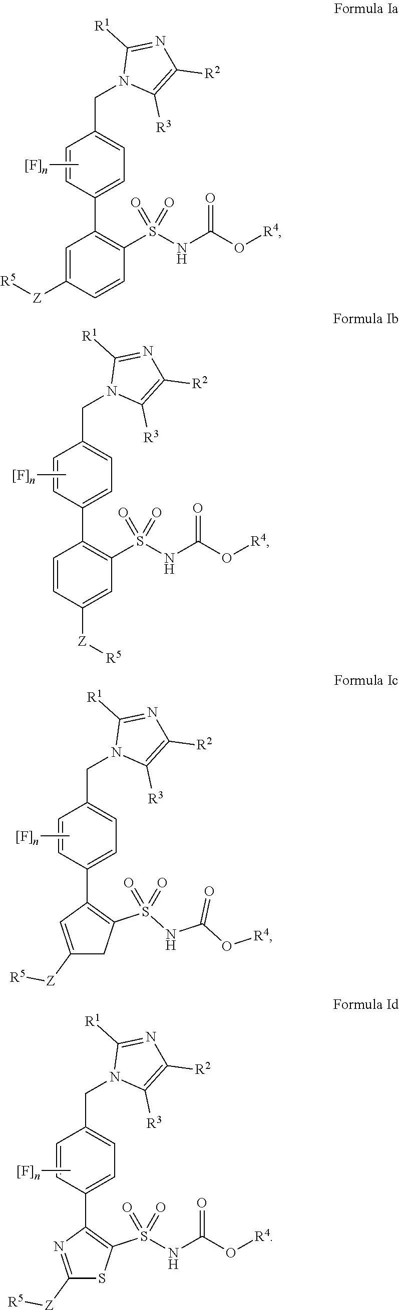

3. A compound as claimed any one of the preceding claims, wherein R¹ represents C₂-C₆ alkyl substituted with 0, 1 or 2 substituents selected from the group consisting of OR⁶, SR⁷, NR⁸R⁹, halogen, thiazole, oxazole and pyrazole.

4. A compound as claimed in any one of the preceding claims, wherein R² and R³ independently represent H.

5. A compound as claimed in claims 3 and 4, wherein R¹ is tert-butyl, isopropyl or cyclopropyl.

6. A compound as claimed in claims 3 and 4, wherein R¹ is C₂-C₆ alkyl substituted by an —OH group or a F atom.

7. A compound as claimed in claim 6, wherein R¹ is 2-hydroxyprop-2-yl, 1-ethanol or 2-fluoroprop-2-yl.

8. A compound as claimed in any one of the preceding claims, wherein R⁴ is selected from the group consisting of methyl, ethyl, n-propyl, isopropyl, n-butyl, isobutyl, sec-butyl, and tert-butyl.

9. A compound as claimed in claim 8, wherein R⁴ is methyl or n-butyl.

10. A compound as claimed in any one of the preceding claims, wherein R⁵ is selected from the group consisting of ethyl, n-propyl, isopropyl, n-butyl, isobutyl, sec-butyl and tert-butyl.

11. A compound as claimed in claim 10, wherein R⁵ is isopropyl or isobutyl.

12. A compound as claimed in any one of the preceding claims, wherein n is 0.

13. A compound as claimed in any one of the preceding claims, wherein Z is a single bond.

14. A compound as claimed in any one of the preceding claims, which is one or more of the following:
methyl ((4′-((2-(tert-butyl)-1H-imidazol-1-yl)methyl)-5-isobutyl-[1,1′-biphenyl]-2-yl)sulfonyl)carbamate,
butyl ((4′-((2-(tert-butyl)-1H-imidazol-1-yl)methyl)-5-isobutyl-[1,1′-biphenyl]-2-yl)sulfonyl)carbamate,
methyl ((4-(4-((2-(tert-butyl)-1H-imidazol-1-yl)methyl)phenyl)-2-isobutylthiazol-5-yl)sulfonyl) carbamate,
butyl ((4-(4-((2-(tert-butyl)-1H-imidazol-1-yl)methyl)phenyl)-2-isobutylthiazol-5-yl)sulfonyl) carbamate,
methyl ((4-(4-((2-(tert-butyl)-1H-imidazol-1-yl)methyl)phenyl)-2-propylthiazol-5-yl)sulfonyl)carbamate,
butyl ((4-(4-((2-(tert-butyl)-1H-imidazol-1-yl)methyl)phenyl)-2-propylthiazol-5-yl)sulfonyl) carbamate,
butyl ((4′-((2-(2-hydroxypropan-2-yl)-1H-imidazol-1-yl)methyl)-5-isobutyl-[1,1′-biphenyl]-2-yl)sulfonyl)carbamate,
methyl ((4′-((2-(2-hydroxypropan-2-yl)-1H-imidazol-1-yl)methyl)-5-isobutyl-[1,1′-biphenyl]-2-yl)sulfonyl)carbamate,
butyl ((4′-((2-(tert-butyl)-1H-imidazol-1-yl)methyl)-5-propyl-[1,1′-biphenyl]-2-yl)sulfonyl)carbamate,
methyl ((4′-((2-(tert-butyl)-1H-imidazol-1-yl)methyl)-5-propyl-[1,1′-biphenyl]-2-yl)sulfonyl)carbamate,
butyl ((3-(4-((2-(tert-butyl)-1H-imidazol-1-yl)methyl)phenyl)-5-isobutylthiophen-2-yl)sulfonyl)carbamate,
methyl ((3-(4-((2-(tert-butyl)-1H-imidazol-1-yl)methyl)phenyl)-5-isobutylthiophen-2-yl)sulfonyl)carbamate,
butyl ((4-(4-((2-(2-hydroxypropan-2-yl)-1H-imidazol-1-yl)methyl)phenyl)-2-propyl thiazol-5-yl)sulfonyl)carbamate,
butyl ((4-(4-((2-isopropyl-1H-imidazol-1-yl)methyl)phenyl)-2-propylthiazol-5-yl) sulfonyl)carbamate,
butyl ((2-isobutyl-4-(4-((2-(thiazol-2-yl)-1H-imidazol-1-yl)methyl)phenyl)thiazol-5-yl) sulfonyl)carbamate,
butyl ((4-(4-((2-(2-hydroxypropan-2-yl)-1H-imidazol-1-yl)methyl)phenyl)-2-isobutyl thiazol-5-yl)sulfonyl)carbamate,
butyl ((2-isobutyl-4-(4-((2-isopropyl-1H-imidazol-1-yl)methyl)phenyl)thiazol-5-yl) sulfonyl)carbamate,
methyl ((4-(4-((2-(2-hydroxypropan-2-yl)-1H-imidazol-1-yl)methyl)phenyl)-2-isobutylthiazol-5-yl)sulfonyl)carbamate,
methyl ((2-isobutyl-4-(4-((2-isopropyl-1H-imidazol-1-yl)methyl)phenyl)thiazol-5-yl) sulfonyl)carbamate,
butyl ((2-propyl-4-(4-((2-(thiazol-2-yl)-1H-imidazol-1-yl)methyl)phenyl)thiazol-5-yl) sulfonyl)carbamate,
butyl ((4-(4-((2-(1-hydroxyethyl)-1H-imidazol-1-yl)methyl)phenyl)-2-isobutylthiazol-5-yl)sulfonyl)carbamate,
methyl ((4-(4-((2-(1-hydroxyethyl)-1H-imidazol-1-yl)methyl)phenyl)-2-isobutylthiazol-5-yl)sulfonyl)carbamate,
butyl ((4-(4-((2-(1-hydroxyethyl)-1H-imidazol-1-yl)methyl)phenyl)-2-propylthiazol-5-yl)sulfonyl)carbamate,
methyl ((4-(4-((2-(1-hydroxyethyl)-1H-imidazol-1-yl)methyl)phenyl)-2-propylthiazol-5-yl)sulfonyl)carbamate,
methyl ((4-(4-((2-cyclopropyl-1H-imidazol-1-yl)methyl)phenyl)-2-isobutylthiazol-5-yl)sulfonyl)carbamate,
butyl ((4-(4-((2-cyclopropyl-1H-imidazol-1-yl)methyl)phenyl)-2-isobutylthiazol-5-yl)sulfonyl)carbamate,
methyl ((4-(4-((2-cyclopropyl-1H-imidazol-1-yl)methyl)phenyl)-2-propylthiazol-5-yl)sulfonyl)carbamate,
butyl ((4-(4-((2-cyclopropyl-1H-imidazol-1-yl)methyl)phenyl)-2-propylthiazol-5-yl)sulfonyl)carbamate,
methyl ((4′-((2-(2-hydroxypropan-2-yl)-1H-imidazol-1-yl)methyl)-5-propyl-[1,1′-biphenyl]-2-yl)sulfonyl)carbamate,
butyl ((4′-((2-(2-hydroxypropan-2-yl)-1H-imidazol-1-yl)methyl)-5-propyl-[1,1′-biphenyl]-2-yl)sulfonyl)carbamate,
butyl ((4′-((2-(2-fluoropropan-2-yl)-1H-imidazol-1-yl)methyl)-5-isobutyl-[1,1′-biphenyl]-2-yl)sulfonyl)carbamate,
methyl ((5-isobutyl-4′-((2-(thiazol-2-yl)-1H-imidazol-1-yl)methyl)-[1,1′-biphenyl]-2-yl)sulfonyl)carbamate,
methyl ((4′-((2-(2-fluoropropan-2-yl)-1H-imidazol-1-yl)methyl)-5-isobutyl-[1,1′-biphenyl]-2-yl)sulfonyl)carbamate,
butyl ((5-isobutyl-4′-((2-(thiazol-2-yl)-1H-imidazol-1-yl)methyl)-[1,1′-biphenyl]-2-yl)sulfonyl)carbamate,
methyl ((5-isobutyl-4′-((2-(isopropan-2-yl)-1H-imidazol-1-yl)methyl)-[1,1′-biphenyl]-2-yl)sulfonyl)carbamate,
butyl ((5-isobutyl-4′-((2-(isopropan-2-yl)-1H-imidazol-1-yl)methyl)-[1,1′-biphenyl]-2-yl)sulfonyl)carbamate,
butyl ((5-isobutyl-4′-((2-(cyclopropan-2-yl)-1H-imidazol-1-yl)methyl)-[1,1′-biphenyl]-2-yl)sulfonyl)carbamate,
methyl ((5-isobutyl-4′-((2-(cyclopropan-2-yl)-1H-imidazol-1-yl)methyl)-[1,1′-biphenyl]-2-yl)sulfonyl)carbamate,
butyl ((5-isobutyl-3-(4-(2-(2-hydroxypropan-2-yl)-1H-imidazol-1-yl)methyl)phenyl)-2-yl)sulfonyl)carbamate,
methyl ((5-isobutyl-3-(4-(2-(2-hydroxypropan-2-yl)-1H-imidazol-1-yl)methyl)phenyl)-2-yl)sulfonyl)carbamate,
butyl ((5-isobutyl-3-(4-(2-(2-fluoropropan-2-yl)-1H-imidazol-1-yl)methyl)phenyl)-2-yl)sulfonyl)carbamate,
methyl ((5-isobutyl-3-(4-(2-(2-fluoropropan-2-yl)-1H-imidazol-1-yl)methyl)phenyl)-2-yl)sulfonyl)carbamate,
2-methoxyethyl ((5-isobutyl-3-(4-(2-(2-tertbutyl)-1H-imidazol-1-yl)methyl)phenyl)-2-yl)sulfonyl)carbamate,
butyl ((4-(4-((2-(2-fluoropropan-2-yl)-1H-imidazol-1-yl)methyl)phenyl)-2-propyl thiazol-5-yl)sulfonyl)carbamate,
methyl ((4-(4-((2-(2-fluoropropan-2-yl)-1H-imidazol-1-yl)methyl)phenyl)-2-propyl thiazol-5-yl)sulfonyl)carbamate,
butyl ((4-(4-((2-(2-fluoropropan-2-yl)-1H-imidazol-1-yl)methyl)phenyl)-2-isobutylthiazol-5-yl)sulfonyl)carbamate,
methyl ((4-(4-((2-(2-fluoropropan-2-yl)-1H-imidazol-1-yl)methyl)phenyl)-2-isobutylthiazol-5-yl)sulfonyl)carbamate,
butyl ((4′-((2-(tert-butyl)-1H-imidazol-1-yl)methyl)-3′-fluoro-5-isobutyl-[1,1′-biphenyl]-2-yl)sulfonyl)carbamate, or
methyl (4′-((2-(tert-butyl)-1H-imidazol-1-yl)methyl)-3′-fluoro-5-isobutyl-[1,1′-biphenyl]-2-yl)sulfonylcarbamate.

15. A compound as defined in any one of claims 1 to 14, for use as a medicament.

16. A pharmaceutical composition comprising a therapeutically effective amount of a compound as claimed in any one of claims 1 to 14, together with at least one pharmaceutically acceptable carrier, excipient and/or diluent.

17. A compound as defined in any one of the preceding claims, for treating and/or preventing a disease, disorder or condition associated with angiotensin II wherein said compound exhibits an acceptable level of CYP inhibition of one or more CYPs.

18. A compound as defined in claim 17, for use in the treatment and/or prevention of a disease, disorder and/or condition selected from the group consisting of hypertension, heart failure, stroke, chronic kidney disease, nephropathy, pulmonary fibrosis such as idiopathic pulmonary fibrosis, sclerosis such as systemic sclerosis, sarcoidosis such as pulmonary sarcoidosis, obstructive lung diseases, such as chronic obstructive lung disease, autoimmune diseases, such as rheumatoid arthritis, viral respiratory tract infections and pneumonia as a consequence thereof, and any combination thereof.

19. The use of a compound as defined in claim 17, for the manufacture of a medicament for the treatment of a disease, disorder and/or condition selected from the group consisting of hypertension, heart failure, stroke, chronic kidney disease, nephropathy, pulmonary fibrosis such as idiopathic pulmonary fibrosis, sclerosis such as systemic sclerosis, sarcoidosis such as pulmonary sarcoidosis, obstructive lung diseases, such as chronic obstructive lung disease, autoimmune diseases, such as rheumatoid arthritis, viral respiratory tract infections and pneumonia as a consequence thereof, and any combination thereof.

20. A method of treatment of disease, disorder and/or condition selected from the group consisting of hypertension, heart failure, stroke, chronic kidney disease, nephropathy, pulmonary fibrosis such as idiopathic pulmonary fibrosis, sclerosis such as systemic sclerosis, sarcoidosis such as pulmonary sarcoidosis, obstructive lung diseases, such as chronic obstructive lung disease, autoimmune diseases, such as rheumatoid arthritis, viral respiratory tract infections and pneumonia as a consequence thereof, which comprises administering a compound as defined in any one of claims 1 to 14 to a patient in need of such treatment.

21. A compound for use as claimed in claim 18, a use as claimed in claim 19, or a method of treatment as claimed in claim 20, wherein the disease is an interstitial lung disease.

22. A compound for use, a use, or a method of treatment as claimed in claim 21, wherein the interstitial lung disease is idiopathic pulmonary fibrosis or sarcoidosis.

23. A compound for use as claimed in claim 18, a use as claimed in claim 19, or a method of treatment as claimed in claim 20, wherein the diseases are heart failure, chronic kidney disease, rheumatoid arthritis and virally-induced pneumonia.

24. A pharmaceutical formulation comprising a compound as defined in any one of claims 1 to 14, a therapeutic agent that is known to be metabolized by a CYP enzyme; and a pharmaceutically-acceptable adjuvant, diluent or carrier.

25. A kit of parts comprising:
(A) A pharmaceutical formulation including a compound as defined in any one of claims 1 to 14 in admixture with a pharmaceutically-acceptable adjuvant, diluent or carrier; and
(B) A pharmaceutical formulation including a therapeutic agent that is known to be metabolized by a CYP enzyme, in admixture with a pharmaceutically-acceptable adjuvant, diluent or carrier,
which components (A) and (B) are each provided in a form that is suitable for administration in conjunction with the other.

26. A formulation as claimed in claim 24, or a kit of parts as claimed in claim 25, wherein the therapeutic agent is selected from the group consisting of pirfenidone, naproxen, propranolol, riluzole, tizanidine, warfarin, celecoxib, clopidogrel, irbesartan, meloxicam, piroxicam, torsemide, cyclophosphamide, indomethacin, atorvastatin, cilostazol, cyclosporine, deflazacort, hydrocortisone, lidocaine, selexipag, sildenafil and/or simvastatin.
A process for the preparation of a compound of Formula I as defined in any one of the preceding claims, by reaction of a compound of Formula 5, 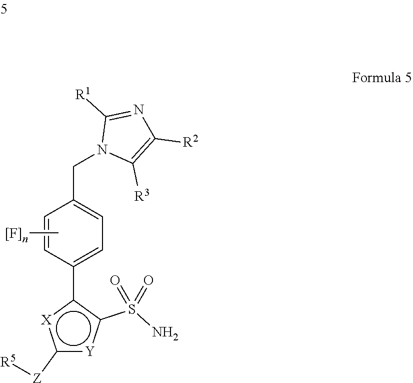
wherein R¹, R², R³, R⁵, X, Y, Z and n are as defined in claim 1 with a compound of Formula 6,
R⁴OC(O)X (Formula 6)
wherein X represents a suitable leaving group, such as halo (e.g. chloro), and R⁴ is as defined in claim 1, optionally following deprotection of a compound of Formula 4, 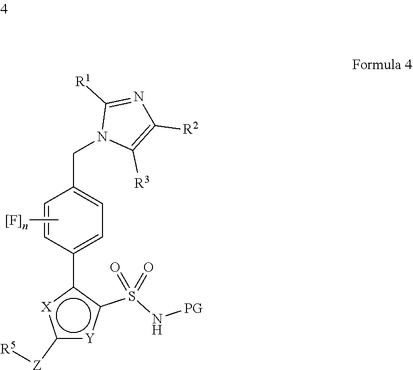
wherein PG represents a protecting group and R¹, R², R³, R⁵, X, Y, Z and n are as defined above to yield a compound of Formula 5 as an intermediate.
